(19) United States
(12) Patent Application Publication
(10) Pub. No.: US 2011/0052697 A1
(43) Pub. Date: Mar. 3, 2011

(54) Aptamer-Directed Drug Delivery

(75) Inventors: Omid C. Farokhzad, Chestnut Hill, MA (US); Sangyong Jon, Gwangju (KR); Vaishali Bagalkot, Gwangju (KR); Liangfang Zhang, Cambridge, MA (US); Benjamin Teply, Omaha, NE (US); Etgar Levy-Nissenbaum, Cambridge, MA (US); Robert S. Langer, Newton, MA (US)

(73) Assignees: GWANGJU INSTITUTE OF SCIENCE & TECHNOLOGY, Gwangju (KR); MASSACHUSETTS INSTITUTE OF TECHNOLOGY, Cambridge, MA (US); THE BRIGHAM AND WOMEN'S HOSPITAL, INC., Boston, MA (US)

(21) Appl. No.: 12/301,225

(22) PCT Filed: May 17, 2007
(86) PCT No.: PCT/US07/69144

§ 371 Date: Nov. 8, 2010

Related U.S. Application Data
(60) Provisional application No. 60/801,007, filed on May 17, 2006.

(51) Int. Cl. A61K 9/14; C07H 21/04; A61K 31/7088; A61P 35/00

(52) U.S. Cl. 424/486; 536/23.1; 514/44 R

(57) ABSTRACT

The present invention provides systems, methods, and compositions for targeted delivery of a therapeutic agent organs, tissues, cells, extracellular matrix components, and intracellular compartments. The present invention provides a complex comprising a therapeutic or diagnostic agent and a nucleic acid targeting moiety, wherein the agent non-covalently associates with base pairs of the nucleic acid targeting moiety. The invention provides targeted particles comprising a particle and an inventive complex. The present invention provides methods of designing, manufacturing, and using inventive complexes and targeted particles.

### RELATED APPLICATIONS

The present application is related to and claims priority under 35 U.S.C. §119(e) to United States provisional patent application, U.S. Ser. No. 60/801,007, filed May 17, 2006 (the '007 application). The entire contents of the '007 application are incorporated herein by reference.

### GOVERNMENT SUPPORT

The United States Government has provided grant support utilized in the development of the present invention. In particular, National Institutes of Health/National Cancer Institute (contract number CA 119349); National Institutes of Health/National Institute of Biomedical Imaging and BioEngineering (contract number EB 003647); and Korea Science and Technology Foundation grant R01-2006-000-10818-0 have supported development of this invention. The United States Government may have certain rights in the invention.

### BACKGROUND OF THE INVENTION

Over one million people are diagnosed with cancer each year. Approximately one out of every two American men and one out of every three American women will have some type of cancer at some point during their lifetimes. Cancer can strike at any age; however, about 77% of all cancers are diagnosed in people age 55 and older (American Cancer Society).

Most cancers are typically treated by a combination of approaches, including surgical removal of a tumor, chemotherapy, and/or radiation therapy. Surgical procedures are usually not sufficient to remove a tumor in its entirety, so surgery is frequently accompanied by chemotherapy and/or radiation therapy. Chemotherapy involves the use of pharmaceutical agents to selectively kill tumor cells, and radiation therapy involves treatment with high-energy rays (e.g., x-rays) to kill tumor cells.

Unfortunately, however, chemotherapy and radiation cause serious and sometimes life-threatening side effects, including fatigue; nausea; vomiting; pain; hair loss; anemia; central nervous system problems; infection; blood clotting problems; mouth, gum, and throat problems; diarrhea; constipation; nerve and muscle effects; kidney and bladder effects; flu-like symptoms; fluid retention; and effects on sexual organs.

Chemotherapy often causes such severe side effects because the treatment involves the systemic administration of cytotoxic agents to a patient. These agents cannot distinguish tumor cells from normal cells and, therefore, kill healthy cells as well as tumor cells. Side effects are worsened because a very large dose must be administered to the patient in order to deliver a therapeutically effective dose to a tumor site. In addition, chemotherapy is often administered to a patient in the form of a therapeutic “cocktail” of multiple chemotherapeutic agents. Administration of multiple drugs at once can increase the severity and duration of adverse side effects. Although radiation therapy is administered somewhat more locally than chemotherapy, radiation treatment still results in the destruction of normal tissue in the vicinity of the tumor.

Thus, targeting of a therapeutic agent (e.g., to a particular tissue or cell type; to a specific diseased tissue but not to normal tissue; etc.) is desirable in the treatment of diseases such as cancer. For example, in contrast to systemic delivery of a cytotoxic anti-cancer agent, targeted delivery could prevent the agent from harming healthy cells. Additionally, targeted delivery may allow for the administration of a lower dose of the chemotherapeutic agent, which could reduce undesirable side effects commonly associated with traditional chemotherapy.

Therefore, a strong need in the art remains for systems that selectively deliver therapeutic agents to targeted organs, tissues, or cells. The ability to control the precise level and location of a therapeutic agent in a patient would allow for reduced dosages of the agent to be administered and reduced side effects. There is a need in the art for improved methods of detecting tumors (e.g., thorough and/or early detection of tumors or cancerous cells).

### SUMMARY OF THE INVENTION

The present invention provides systems for selectively delivering therapeutic or diagnostic agents to particular organs, tissues, cells, extracellular matrix components, and/or intracellular compartments using a nucleic acid targeting moiety for targeting. In certain embodiments, therapeutic or diagnostic agents are specifically delivered to diseased tissues based on targeting directed by nucleic acid targeting moieties. In certain specific embodiments, therapeutic or diagnostic agents are specifically delivered to tumors (e.g. malignant tumors or benign tumors).

In one aspect, the present invention provides a complex comprising a nucleic acid targeting moiety (e.g. an aptamer or spiegelmer) and a therapeutic or diagnostic agent that is non-covalently associated with the base pairs of the nucleic acid targeting moiety.

In some embodiments, complexes useful in accordance with the present invention comprise a nucleic acid targeting moiety which specifically binds to one or more targets associated with an organ, tissue, cell, extracellular matrix component, and/or intracellular compartment. As used herein, the terms “target” and “marker” can be used interchangeably.

A nucleic acid targeting moiety may be an aptamer, which is generally an oligonucleotide (e.g., DNA, RNA, or an analog or derivative thereof) that binds to a particular target, such as a polypeptide, carbohydrate, or other target. In general, the targeting function of the aptamer is based on the three-dimensional structure of the aptamer, not exclusively on its primary sequence. Binding of an aptamer to a target is typically mediated by the interaction between the two- and/or three-dimensional structures of both the aptamer and the target. Binding of an aptamer to a target is typically not solely based on the primary sequence of the aptamer, but depends on the three-dimensional structure(s) of the aptamer and/or target. In some embodiments, aptamers may bind to their targets via complementary Watson-Crick base pairing which is interrupted by structures (e.g. hairpin loops) that disrupt base pairing. In some embodiments, nucleic acid targeting moieties are spiegelmers (i.e. mirror image aptamers).

In some embodiments, a target may be a marker that is exclusively or primarily associated with one or a few organs, with one or a few tissue types, with one or a few cell types, with one or a few diseases, and/or with one or a few developmental stages. In some embodiments, a target can be a protein (e.g. cell surface receptor, transmembrane protein, glycoprotein, etc.), a carbohydrate (e.g. glycan moiety, glycocalyx, etc.), a lipid (e.g. steroid, phospholipid, etc.), and/or a nucleic acid (e.g. DNA, RNA, etc.). In some embodiments, a target (i.e. marker) is a molecule that is present exclusively or in higher amounts on a malignant cell, e.g., a tumor antigen.

In one aspect, the present invention provides targeted particles comprising a particle and a complex, wherein the complex comprises a nucleic acid targeting moiety (e.g. an aptamer or spiegelmer) and a therapeutic or diagnostic agent to be delivered. In general, the particle is delivered to an organ, tissue, cell, extracellular matrix component, and/or intracellular compartment that is associated with a target which is able to bind to the nucleic acid targeting moiety. The agent is delivered once the target binds to the nucleic acid targeting moiety. According to the present invention, the agent to be delivered is non-covalently associated with the base pairs of the aptamer or spiegelmer and is released from the nucleic acid targeting moiety upon binding to the target. In certain embodiments, the therapeutic or diagnostic agent is intercalated between the base pairs of the nucleic acid targeting moiety.

Any particle can be used in accordance with the targeted particles of the present invention. In some embodiments, particles are biodegradable and biocompatible. In general, a particle useful in accordance with the present invention is any entity having a greatest dimension (e.g. diameter) of less than 100 microns (μm). In some embodiments, particles have a greatest dimension of less than 10 μm. In some embodiments, particles have a greatest dimension of less than 1000 nanometers (nm). In some embodiments, particles are spheres, spheroids, flat, plate-shaped, cubes, cuboids, ovals, ellipses, cylinders, cones, or pyramids. In some embodiments, particles are microparticles (e.g. microspheres). In some embodiments, particles are nanoparticles (e.g. nanospheres). In some embodiments, particles are liposomes. In some embodiments, particles are micelles. Particles can be solid or hollow and can comprise one or more layers (e.g., nanoshells, nanorings).

In some embodiments, particles can comprise a polymeric matrix. In some embodiments, a complex comprising a therapeutic or diagnostic agent to be delivered and a nucleic acid targeting moiety can be associated with the surface of, encapsulated within, surrounded by, and/or dispersed throughout a polymeric matrix. The polymer of the matrix of particles may be a natural or synthetic polymer.

In some embodiments, a polymeric matrix is made of polyalkenes, polycarbonates, polyanhydrides, polyhydroxyacids, polyfumarates, polycaprolactones, polyamides, polyacetals, polyethers, polyesters, poly(orthoesters), polyvinyl alcohols, polyurethanes, polyphosphazenes, polyacrylates, polymethacrylates, polycyanoacrylates, polyureas, polystyrenes, and/or polyamines. In some embodiments, a polymeric matrix may comprise poly(lactic acid) (PLA), poly(glycolic acid) PGA, poly(lactic-co-glycolic acid) (PLGA), polyethylene glycol (PEG), and/or copolymers thereof.

In some embodiments, particles can be non-polymeric particles (e.g. metal particles, quantum dots, ceramics, inorganic materials, bone-derived materials, bone substitutes, etc.). In some embodiments, a complex of an aptamer or spiegelmer and agent can be covalently associated with a non-polymeric particle. In some embodiments, a complex of an aptamer or spiegelmer and agent can be non-covalently associated with a non-polymeric particle. In some embodiments, an inventive complex can be associated with the surface of, encapsulated within, surrounded by, and/or dispersed throughout a non-polymeric polymer.

Inventive targeted particles may be manufactured using any available method. Typically, the method allows for the preparation of an inventive targeted particle comprising a nucleic acid targeting moiety. In some embodiments, inventive complexes are covalently associated with the particle. In some embodiments, complexes are not covalently associated with a particle. Inventive complexes can be released by diffusion, degradation of the particle, and/or combination thereof.

Physical association can be achieved in a variety of different ways. Physical association may be covalent or non-covalent and may or may not involve a cross-linking step. The particle and complex may be directly associated with one another, e.g., by one or more covalent bonds, or the association may be mediated by one or more linkers.

In one aspect, the invention provides methods of using inventive complexes or targeted particles to treat, alleviate, ameliorate, relieve, delay onset of, inhibit progression of, reduce severity of, and/or reduce incidence of one or more symptoms or features of a disease, disorder, and/or condition (e.g., autoimmune disorders; inflammatory disorders; infectious diseases; neurological disorders; cardiovascular disorders; proliferative disorders; respiratory disorders; digestive disorders; musculoskeletal disorders; endocrine, metabolic, and nutritional disorders; urological disorders; psychological disorders; skin disorders; blood and lymphatic disorders; etc.). In certain embodiments, inventive complexes or targeted particles may be used to treat cancer (e.g. prostate cancer, lung cancer, breast cancer, colorectal cancer, bladder cancer, pancreatic cancer, endometrial cancer, ovarian cancer, bone cancer, esophageal cancer, liver cancer, stomach cancer, brain tumors, cutaneous melanoma, leukemia, just to name a few). The compositions of the present invention may be administered by any route of administration effective for treatment.

In some embodiments, targeted particles may comprise at least a second therapeutic or diagnostic agent (e.g. one that is useful for treatment, prophylaxis, and/or diagnosis of a disease, disorder, and/or condition) that is encapsulated within the polymeric matrix of a particle. According to the present invention, any agents, including, for example, therapeutic agents (e.g. anti-cancer agents), diagnostic agents (e.g. contrast agents; radionuclides; metals; fluorescent, luminescent, and magnetic moieties), prophylactic agents (e.g. vaccines), and/or nutraceutical agents (e.g. vitamins, minerals, etc.) may be delivered. Exemplary agents to be delivered in accordance with the present invention include, but are not limited to, small molecules (e.g. cytotoxic agents, antibiotics), nucleic acids (e.g. DNA, RNAi-inducing entities), proteins (e.g. antibodies), lipids, carbohydrates, hormones, metals, radioactive elements and compounds, drugs, vaccines, immunological agents, etc., and/or combinations thereof. In some embodiments, the agent to be delivered is an agent useful in the treatment of cancer. In some embodiments, the agent to be delivered may be a combination of pharmaceutically active agents. In some embodiments, the agent to be delivered may be a combination of anti-cancer agents. In some embodiments, inventive targeted particles are administered in combination with one or more of the anti-cancer agents described herein.

Inventive therapeutic protocols involve administering a therapeutically effective amount of an inventive complex or targeted particle to a subject who is susceptible to a disease, disorder, and/or condition, such that the disease is prevented or such that the onset of the disease is delayed. In some embodiments, inventive complexes or targeted particles may be administered to a subject who is susceptible to cancer (e.g., patients who have a family history of cancer; patients carrying one or more genetic mutations associated with development of cancer; patients infected by a virus associated with development of cancer; patients with habits and/or lifestyles associated with development of cancer; etc.), such that cancer is prevented or such that the onset of cancer is delayed.

In some embodiments, targeted particles of the present invention may be used to diagnose a disease, disorder, and/or condition. In some embodiments, inventive targeted particles may be used to diagnose cancer. In some embodiments, such methods of diagnosis may involve the use of inventive targeted particles to physically detect and/or locate a tumor within the body of a subject. In some embodiments, inventive targeted particles comprise particles which have intrinsically detectable properties (e.g. quantum dots, magnetic particles, radioisotopes, etc.). In some embodiments, inventive targeted particles comprise particles which do not have intrinsically detectable properties but are associated with a substance which is detectable (e.g. fluorescent or radioactive moiety). Such targeted particles are capable of simultaneously diagnosing and treating cancer. In particular, such targeted particles are capable of treating cancer by delivery of the therapeutic or diagnostic agent that is intercalated between the base pairs of the nucleic acid targeting moiety, and such targeted particles are capable of diagnosing cancer by delivery of a detectable particle to the site of a tumor.

In one aspect, the present invention provides kits useful for carrying out various aspects of the invention. In some embodiments, a kit may include, for example, (i) a complex comprising a nucleic acid targeting moiety and one or more therapeutic or diagnostic agents that are non-covalently associated with the base pairs of the nucleic acid targeting moiety; and (ii) instructions for administering the inventive complex to a subject in need thereof. In some embodiments, a kit may include (i) a targeted particle comprising a particle and a complex, wherein the complex comprises an aptamer or spiegelmer and one or more therapeutic or diagnostic agents that are capable of intercalating between the base pairs of the aptamer or spiegelmer; and (ii) instructions for administering the inventive particle to a subject in need thereof.

This application refers to various issued patents, published patent applications, journal articles, and other publications, all of which are incorporated herein by reference.

### BRIEF DESCRIPTION OF THE DRAWING

FIG. 1: (A) Physical-conjugate formation between an aptamer and a model drug. (B) 2D structure of the A10 PSMA aptamer as predicted by M fold program and the chemical structure of doxorubicin.

FIG. 2: Fluorescence spectra of doxorubicin solution (1.5 mm) with increasing molar ratios of the aptamer (from top to bottom: 0, 0.01, 0.03, 0.1, 0.3, 0.5, 1, 3, 5, 7, and 10 equiv). Inset: A Hill plot for the aptamer titration (K_{d}=0.6 mm; 0.52 equiv of the aptamer).

FIG. 3: Time dependent release profile of the aptamer-Dox complex (closed circle) and Dox without aptamer (closed triangle); n=3.

FIG. 4: Confocal laser scanning microscopy images (superimposed images of fluorescence and transmittance) of LNCaP (A, C) and PC3 (B, D) cells after treatments of 1.5 mm free doxorubicin (A, B) and of 1.5 mm Apt-Dox physical conjugate (C, D) for 2 hours. Scale bars: 20 mm.

FIG. 5: Aptamer cell binding assay. LNCaP cells were incubated with aptamer at a saturating concentration and with aptamer-Dox complexes. Cell-bound aptamer and aptamer-Dox complexes were recovered from cells, purified, and subjected to RT-PCR amplification: lane 1=100 bp DNA ladder; lane 2=free aptamer; lane 3=bound aptamer from LNCaP cell; lane 4=bound aptamer-Dox complex from LNCaP cell.

FIG. 6: Flow cytometry histogram profiles of LNCaP (dotted line) and PC3 (solid line) cells obtained after treatments with (A) nothing, (B) 1.5 mm free doxorubicin, and (C) 1.5 mm Apt-Dox physical conjugate. FL2 log=fluorescence intensity of FL2 sensor (band pass filter, 575 nm).

FIG. 7: Growth-inhibition assay (MTT) results for prostrate cancer cell lines LNCaP and PC3 after 2 hours of incubation with free doxorubicin (5 mm) and the physical conjugate (5 mm) and 24 hours of subsequent incubation. * indicates the LNCaP result that is significantly different from that with PC3 cells (p<0.005, n=5).

FIG. 8: Schematic illustration of (A) the intercalation of a hydrophilic anthracycline drug, such as doxorubicin (Dox), within the A10 PSMA aptamer; (B) the encapsulation of a hydrophobic drug, such as docetaxel (Dtxl), within PLGA-b-PEG nanoparticles using the nanoprecipitation method; and (C) nanoparticle-aptamer (NP-Apt) targeted particles comprising PLGA-b-PEG nanoparticles surface functionalized with the A10 PSMA aptamer for co-delivery of Dtxl and Dox. Both drugs can be released from the targeted particles over time.

FIG. 9: Drug release of docetaxel (black squares) and doxorubicin (red circles) from NP-Apt targeted particles at 37° C. in PBS measured by HPLC. The average molar ratio of Dtxl:Dox carried by each targeted particle is 9:1.

FIG. 10: Binding of PSMA targeted nanoparticle-aptamer targeted particles to LNCaP (+PSMA) and PC3 (−PSMA) prostate epithelial cells. The data demonstrate that NBD (green), serving as an analog of hydrophobic drug encapsulated within the nanoparticles, and Dox (red) serving as a model hydrophilic anthracycline drug intercalated within aptamers were both selectively delivered to LNCaP cells which express the PSMA protein (left panel), but not PC3 cells which do not express the PSMA protein (right panel). The dim red fluorescence in PC3 cells may be due to small amount of doxorubicin released from the targeted particles during incubation with the cells. Note that Dox can diffuse through cell membranes.

FIG. 11: MTT assay to measure the cytotoxicity of NP-Apt targeted particles carrying both Dtxl and Dox [NP(Dtxl)-Apt(Dox)]; Dtxl alone [NP(Dtxl)-Apt]; Dox alone [NP-Apt(Dox)]; or no drug (NP-Apt) to LNCaP and PC3 cell lines. NP-Apt targeted particles were incubated with cells for 6 hours, and cells were subsequently washed and incubated in media for a total of 72 hours before assessing cell viability in each group (n=4). *denotes statistical significance by one-sided two-sample T-test with equal variances (p=0.029).

FIG. 12: (A) Schematic illustration of QD-Apt-Dox targeted particles comprising a CdSe/ZnS core-shell QD surface functionalized with A10 PSMA aptamers into which doxorubicin (Dox) is intercalated. QD-Apt-Dox targeted particles form a bi-FRET system, in which Dox quenches fluorescence of the QD, and Dox fluorescence is quenched by the aptamer. (B) Schematic illustration of specific endocytic uptake of QD-Apt-Dox targeted particles into target cancer cells. Dox release from QD-Apt-Dox targeted particles induces fluorescence recovery of both QD and Dox, thereby enabling synchronous cancer imaging and therapy.

FIG. 13: Gel electrophoresis results of QD-Apt targeted particles (A) before staining with ethidium bromide, and (B) after staining with ethidium bromide. Lanes 1, 2, 3, and 4 represent 100 bp ladder, Apt only, QD-Apt targeted particle, and QD only, respectively.

FIG. 14: Fluorescence spectra of (A) QD in QD-Apt targeted particle solution (0.1 nM) with increasing molar ratios of Dox (from top to bottom: 0, 0.1, 0.3, 0.6, 1, 1.5, 2.1, 2.8, 3.5, 4.5, 5.5, 7.0, and 8.0) at an excitation of 350 nm, and (B) Dox solution (10 μM) with increasing molar ratios of QD-Apt targeted particle (from top to bottom: 0.02, 0.04, 0.07, 0.09, 0.12, 0.14, and 0.16) at an excitation of 480 nm.

FIG. 15: Binding of PSMA targeted QD-Apt targeted particles to (A) LNCaP (+PSMA), and (B) PC3 (−PSMA) prostate epithelial cells.

FIG. 16: Confocal laser scanning microscopy images of PSMA-expressing LNCaP cells after being incubated with 100 nM QD-Apt targeted particles for 0.5 hours at 37° C., washed two times in PBS, and further incubated at 37° C. for (A) 0 hours, and (B) 1.5 hours.

FIG. 17: MTT assay to measure the cytotoxicity of QD alone (100 nM), Dox alone (5 μM), and QD-Apt targeted particles (QD: 100 nM) to LNCaP and PC3 cell lines. Particulates were incubated with cells for 3 hours, and cells were subsequently washed and incubated in media for 24 hours before assessing cell viability in each group (n=3). * indicates that the LNCaP result is significantly different from PC3 cells (p<0.005).

### DEFINITIONS

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid. In some embodiments, an amino acid is a D-amino acid. In some embodiments, an amino acid is an L-amino acid. As used herein, “natural amino acid” refers to any of the twenty standard L-amino acids commonly found in naturally-occurring peptides. As used herein, “unnatural amino acid” encompasses any amino acid other than the 20 natural amino acids. Unnatural amino acids may be chemically produced or modified amino acids, including but not limited to salts and/or amino acid derivatives (such as amides). Amino acids, including carboxy- and/or amino-terminal amino acids in peptides, can be modified by methylation, amidation, acetylation, and/or substitution with other chemical groups. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Aryl: As used herein, the terms “aryl” and “heteroaryl” generally refer to stable mono- or poly-cyclic, heterocyclic, polycyclic, and polyheterocyclic unsaturated moieties having preferably 3-14 carbon atoms, each of which may be substituted or unsubstituted. Substituents include, but are not limited to, any of the previously mentioned substituents, i.e., the substituents recited for aliphatic moieties, or for other moieties as disclosed herein, resulting in the formation of a stable compound. In certain embodiments of the present invention, “aryl” refers to a mono- or bi-cyclic carbocyclic ring system having one or two aromatic rings including, but not limited to, phenyl, naphthyl, tetrahydronaphthyl, indanyl, indenyl, and the like. In certain embodiments of the present invention, the term “heteroaryl,” as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ (CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

Associated with: As used herein, the term “associated with” refers to the state of two or more entities which are linked by a direct or indirect covalent or non-covalent interaction. In some embodiments, an association is covalent. In some embodiments, a covalent association is mediated by a linker moiety. In some embodiments, an association is non-covalent (e.g. charge interactions, affinity interactions, metal coordination, physical adsorption, host-guest interactions, hydrophobic interactions, Π stacking interactions, hydrogen bonding interactions, van der Waals interactions, magnetic interactions, electrostatic interactions, dipole-dipole interactions, etc.). For example, in some embodiments, a complex (e.g. nucleic acid targeting moiety and an agent capable of intercalating between the base pairs of the nucleic acid targeting moiety) is covalently associated with a particle. In some embodiments, a complex (e.g. nucleic acid targeting moiety and an agent capable of intercalating between the base pairs of the nucleic acid targeting moiety) is non-covalently associated with a particle, (e.g. the complex may be associated with the surface of, encapsulated within, surrounded by, and/or distributed throughout a polymeric matrix of a particle).

Biocompatible: As used herein, the term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo.

Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Cell type: As used herein, the term “cell type” refers to a form of cell having a distinct set of morphological, biochemical, and/or functional characteristics that define the cell type. One of skill in the art will recognize that a cell type can be defined with varying levels of specificity. For example, prostate endothelial cells and circulatory system endothelial cells are distinct cell types, which can be distinguished from one another but share certain features that are characteristic of the broader “endothelial” cell type of which both are members. Typically, cells of different types may be distinguished from one another based on their differential expression of a variety of genes which are referred to in the art as “markers” of a particular cell type or types (e.g., cell types of a particular lineage). A “cell type specific marker” is a gene product or modified version thereof that is expressed at a significantly greater level by one or more cell types than by all or most other cell types and whose expression is characteristic of that cell type. Many cell type specific markers are recognized as such in the art. Similarly, a “tissue specific marker” is one that is expressed at a significantly greater level by cells of a type that is characteristic of a particular tissue than by cells that are characteristic of most or all other tissues.

Characteristic portion: As used herein, the phrase a “characteristic portion” of a substance, in the broadest sense, is one that shares some degree of sequence and/or structural identity and/or at least one functional characteristic with the relevant intact substance. For example, a “characteristic portion” of a polynucleotide is one that contains a continuous stretch of nucleotides, or a collection of continuous stretches of nucleotides, that together are characteristic of a polynucleotide. In some embodiments, each such continuous stretch generally will contain at least 2, 5, 10, 15, 20 or more nucleotides. In some embodiments, the characteristic portion may be biologically active.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g. between nucleic acid molecules (e.g. DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95% or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using a NWSgapdna.CMP matrix.

In vitro: As used herein, the term “in vitro” refers to events that occur in an artificial environment, e.g., in a test tube or reaction vessel, in cell culture, etc., rather than within an organism (e.g. animal, plant, and/or microbe). As used herein, “in vitro” can be used to describe a microorganism in culture.

In vivo: As used herein, the term “in vivo” refers to events that occur within an organism (e.g. animal, plant, and/or microbe).

Nucleic acid: As used herein, the term “nucleic acid,” in its broadest sense, refers to any compound and/or substance that can be incorporated into an oligonucleotide chain. As used herein, the terms “nucleic acid” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. The term “nucleic acid sequence” as used herein can refer to the nucleic acid material itself and is not restricted to the sequence information (e.g. the succession of letters chosen, for example, among the five base letters A, G, C, T, or U) that biochemically characterizes a specific nucleic acid, e.g., a DNA or RNA molecule. A nucleic acid sequence is presented in the 5′ to 3′ direction unless otherwise indicated. The term “nucleic acid segment” is used herein to refer to a nucleic acid sequence that is a portion of a longer nucleic acid sequence. In some embodiments, a “nucleic acid” or “polynucleotide” comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid targeting moiety: As used herein, the term “nucleic acid targeting moiety” refers to any nucleic acid that binds to a component associated with a cell. Such a component is referred to as a “target” or a “marker.” In some embodiments, a nucleic acid targeting moiety is an aptamer that binds to a cell type specific marker. As used herein, an aptamer refers to a polynucleotide that binds to a specific target structure that is associated with a particular organ, tissue, cell, extracellular matrix component, and/or intracellular compartment. In general, the targeting function of the aptamer is based on the three-dimensional structure of the aptamer. In some embodiments, binding of an aptamer to a target is typically mediated by the interaction between the two- and/or three-dimensional structures of both the aptamer and the target. In some embodiments, binding of an aptamer to a target is not solely based on the primary sequence of the aptamer, but depends on the three-dimensional structure(s) of the aptamer and/or target. In some embodiments, aptamers bind to their targets via complementary Watson-Crick base pairing which is interrupted by structures (e.g. hairpin loops) that disrupt base pairing. In some embodiments, nucleic acid targeting moieties are spiegelmers. In general, spiegelmers are synthetic, mirror-image nucleic acids that can specifically bind to a target (i.e. mirror image aptamers). Spiegelmers are characterized by structural features which make them not susceptible to exo- and endo-nucleases.

Particle: As used herein, a “particle” refers to any entity having a diameter of less than 100 microns (μm). Typically, particles have a longest dimension (e.g. diameter) of 1000 nm or less. In some embodiments, particles have a diameter of 300 nm or less. In some embodiments, nanoparticles have a diameter of 200 nm or less. In some embodiments, nanoparticles have a diameter of 100 nm or less. In general, particles are greater in size than the renal excretion limit, but are small enough to avoid accumulation in the liver. In some embodiments, a population of particles may be relatively uniform in terms of size, shape, and/or composition. In general, inventive particles are biodegradable and/or biocompatible. Inventive particles can be solid or hollow and can comprise one or more layers. In some embodiments, particles are spheres, spheroids, flat, plate-shaped, cubes, cuboids, ovals, ellipses, cylinders, cones, or pyramids. In some embodiments, particles can be a matrix of polymers. In some embodiments, the matrix is cross-linked. In some embodiments, formation of the matrix involves a cross-linking step. In some embodiments, the matrix is not substantially cross-linked. In some embodiments, formation of the matrix does not involve a cross-linking step. In some embodiments, particles can be a non-polymeric particle (e.g. a metal particle, quantum dot, ceramic, inorganic material, bone-derived materials, bone substitutes, etc.). Inventive particles may be microparticles, nanoparticles, liposomes, and/or micelles. As used herein, the term “nanoparticle” refers to any particle having a diameter of less than 1000 nm.

Pure: As used herein, a substance and/or entity is “pure” if it is substantially free of other components. For example, a preparation that contains more than about 90% of a particular substance and/or entity is typically considered to be a pure preparation. In some embodiments, a substance and/or entity is at least 91%, 92%, 93%, 94%, 95%, 96%, 97%, 98%, or 99% pure.

Similarity: As used herein, the term “similarity” refers to the overall relatedness between polymeric molecules, e.g. between nucleic acid molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small molecule: In general, a “small molecule” is understood in the art to be an organic molecule that is less than about 2000 g/mol in size. In some embodiments, the small molecule is less than about 1500 g/mol or less than about 1000 g/mol. In some embodiments, the small molecule is less than about 800 g/mol or less than about 500 g/mol. In some embodiments, small molecules are non-polymeric and/or non-oligomeric. In some embodiments, small molecules are not proteins, peptides, or amino acids. In some embodiments, small molecules are not nucleic acids or nucleotides. In some embodiments, small molecules are not saccharides or polysaccharides.

Specific binding: As used herein, the term “specific binding” refers to non-covalent physical association of a first and a second moiety wherein the association between the first and second moieties is at least 10 times as strong, at least 50 times as strong, or at least 100 times as strong as the association of either moiety with most or all other moieties present in the environment in which binding occurs. Binding of two or more entities may be considered specific if the equilibrium dissociation constant, K_{d}, is 10⁻³ M or less, 10⁻⁴M or less, 10⁻⁵M or less, 10⁻⁶M or less, 10⁻⁷M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g. a plurality of individual interactions, wherein each individual interaction is characterized by a K_{d} of greater than 10⁻³M). In some embodiments, specific binding, which can be referred to as “molecular recognition,” is a saturable binding interaction between two entities that is dependent on complementary orientation of functional groups on each entity. Examples of specific binding interactions include aptamer-target interactions, antibody-antigen interactions, avidin-biotin interactions, ligand-receptor interactions, metal-chelate interactions, hybridization between complementary nucleic acids, etc.

Subject: As used herein, the term “subject” or “patient” refers to any organism to which a composition of this invention may be administered, e.g., for experimental, diagnostic, and/or therapeutic purposes. Typical subjects include animals (e.g., mammals such as mice, rats, rabbits, non-human primates, and humans) and/or plants.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (e.g., cancer) may be characterized by one or more of the following: (1) a genetic mutation associated with development of the disease, disorder, and/or condition (e.g. a mutation in an oncogene-encoding gene); (2) a genetic polymorphism associated with development of the disease, disorder, and/or condition (e.g. a polymorphism in the promoter region of an oncogene-encoding gene); (3) increased and/or decreased expression and/or activity of a protein associated with the disease, disorder, and/or condition (e.g. overexpression of the EGF receptor or TGF-α); (4) habits and/or lifestyles associated with development of the disease, disorder, and/or condition (e.g. smoking, obesity, unhealthy diet, lack of exercise); (5) a family history of the disease, disorder, and/or condition (e.g. parent with cancer); (6) infection by a microbe associated with development of the disease, disorder, and/or condition (e.g. infection by a virus such as HPV). In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target: As used herein, the term “target” or “marker” refers to any entity that is capable of specifically binding to a particular nucleic acid targeting moiety. In some embodiments, targets are specifically associated with one or more particular tissue types. In some embodiments, targets are specifically associated with one or more particular cell types. In some embodiments, targets are specifically associated with one or more particular disease states. In some embodiments, targets are specifically associated with one or more particular developmental stages. For example, a cell type specific marker is typically expressed at levels at least 2 fold greater in that cell type than in a reference population of cells. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1000 fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid, as described herein.

Targeted: A substance is considered to be “targeted” for the purposes described herein if it specifically binds to a nucleic acid targeting moiety. In some embodiments, a nucleic acid targeting moiety specifically binds to a target under stringent conditions. An inventive complex or targeted particle comprising a nucleic acid targeting moiety is considered to be “targeted” if the nucleic acid targeting moiety specifically binds to a target, thereby delivering the entire complex or targeted particle composition to a specific organ, tissue, cell, extracellular matrix component, and/or intracellular compartment.

Therapeutically effective amount: As used herein, the term “therapeutically effective amount” means an amount of a therapeutic and/or diagnostic agent (e.g., inventive complex or targeted particle) that is sufficient, when administered to a subject suffering from or susceptible to a disease, disorder, and/or condition, to treat and/or diagnose the disease, disorder, and/or condition.

Therapeutic agent: As used herein, the phrase “therapeutic agent” refers to any agent that, when administered to a subject, has a therapeutic and/or diagnostic effect and/or elicits a desired biological and/or pharmacological effect.

Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an inventive complex or targeted particle to a subject.

### DETAILED DESCRIPTION OF CERTAIN PREFERRED EMBODIMENTS OF THE INVENTION

The present invention provides systems for selectively delivering therapeutic or diagnostic agents to particular organs, tissues, cells, and/or intracellular compartments using a nucleic acid targeting moiety for targeting. In certain embodiments, therapeutic or diagnostic agents are specifically delivered to diseased organs, tissues, cells, and/or intracellular compartments based on targeting directed by nucleic acid targeting moieties. In certain specific embodiments, therapeutic or diagnostic agents are specifically delivered to tumors (e.g. malignant tumors or benign tumors).

The present invention provides a complex comprising a nucleic acid targeting moiety and a therapeutic or diagnostic agent that is non-covalently associated with the base pairs of the nucleic acid targeting moiety. A nucleic acid targeting moiety may be an aptamer, which is generally an oligonucleotide (e.g., DNA, RNA, or an analog or derivative thereof) that binds to a particular target, such as a polypeptide, carbohydrate, or other target. In general, the targeting function of the aptamer is based on the three-dimensional structure of the aptamer, not exclusively on its primary sequence. In some embodiments, nucleic acid targeting moieties are spiegelmers (i.e. mirror image aptamers).

The present invention provides targeted particles comprising a particle and a complex, wherein the complex comprises a nucleic acid targeting moiety (e.g. an aptamer or spiegelmer) and a therapeutic or diagnostic agent to be delivered. In general, the particle is delivered to an organ, tissue, cell, extracellular matrix component, and/or intracellular compartment that is associated with a target which is able to bind to the nucleic acid targeting moiety. The agent is delivered once the target binds to the nucleic acid targeting moiety. According to the present invention, the therapeutic or diagnostic agent to be delivered is non-covalently associated with the base pairs of the aptamer or spiegelmer and is released from the nucleic acid targeting moiety upon binding to the target. In certain embodiments, the agent is intercalated between the base pairs of the nucleic acid targeting moiety.

### Complex of Nucleic Acid Targeting Moiety and Intercalating Agents

Intercalating Agents

According to the present invention, inventive complexes typically comprise one or more nucleic acid targeting moieties and one or more therapeutic or diagnostic agents to be delivered to an organ, tissue, cell, extracellular matrix component, and/or intracellular compartment. According to the present invention, the agent to be delivered may be capable of intercalating between the base pairs of the nucleic acid targeting moiety (i.e. an “intercalating agent,” as used herein). The agent is typically released from the nucleic acid targeting moiety upon delivery of the complex to the target.

In some embodiments, inventive complexes may comprise a nucleic acid which does not behave as a nucleic acid targeting moiety and a therapeutic or diagnostic agent to be delivered to an organ, tissue, cell, extracellular matrix component, and/or intracellular compartment. According to the present invention, the agent to be delivered may be capable of intercalating between the base pairs of any nucleic acid (i.e. an “intercalating agent,” as used herein). The agent is typically released from the nucleic acid targeting moiety upon delivery of the complex to the target. Methods of delivering inventive complexes for which the agent to be delivered is intercalated between the base pairs of a nucleic acid which does not behave as a targeting moiety are described below, in the section entitled “Therapeutic Applications.”

In general, intercalation is the inclusion of a substance (e.g. a molecule) between two other substances (e.g. molecules). In some embodiments, intercalation is a reversible process. A large class of intercalating agents intercalates into a polynucleotide in the space between two adjacent base pairs. Such intercalating agents are typically polycyclic, aromatic, and/or planar. In some embodiments, intercalating agents comprise at least one planar aromatic ring. In certain embodiments, intercalating agents may include aryl or heteroaryl ring systems. That intercalating agents are substantially planar allows them to fit between the base pairs of a polynucleotide. To give but a few examples, known polynucleotide intercalators include ethidium, proflavin, thalidomide, and anthracyclines (e.g. doxorubicin, daunrubicin, epirubicin, idarubicin, mitoxantrone, aclarubicin, pirarubicin, etc.).

In order for an intercalator to fit between base pairs, the base pairs usually need to be separated by at least 0.3 nm, inducing local structural changes to the DNA strand, such as unwinding of the double helix and lengthening of the DNA strand. These structural modifications lead to functional changes, often to the inhibition of transcription and replication processes, which makes some intercalators potent mutagens. DNA intercalators are often carcinogenic, such as benzopyrene diol epoxide, bisbenzimide, aflatoxin, and ethidium bromide.

To give but one example, anthracyclines (e.g. doxorubicin) are intercalating agents that can be used as chemotherapeutic agents, but they are notorious for causing cardiotoxicity. Cardiotoxicity may be caused by many factors, which may include interference with the ryanodine receptor of the sarcoplasmic reticulum in the heart muscle cells, free radical formation in the heart, or from buildup of metabolic products of the anthracycline in the heart. Cardioxicity often presents as EKG changes and arrhythmias, or as a cardiomyopathy leading to congestive heart failure (sometimes presenting many years after treatment). Cardiotoxicity is related to a patient's cumulative lifetime dose of the drug. The present invention encompasses the recognition that targeted delivery of anthracyclines could potentially prevent, inhibit, or delay the onset of cardiotoxicity.

The primary mode of action of anthracyclines (e.g. doxorubicin) is believed to be their reversible binding to nucleolar DNA, which causes inhibition of the replication process (Neidle, 1979, Prog. Med. Chem., 16:151). They also create iron-mediated free radicals that damage DNA and cell membranes. Numerous biochemical studies including evidence from NMR spectroscopic and X-ray crystallographic studies have shown that anthracyclines intercalate into the B-form of the DNA double stranded helix with guanine-cytosine d(CpG) site-specific interactions (Chaires et al., 1990, Biochemistry, 29:2538). As a result of intercalation with anthracyclines (Wang et al., 1987, Biochemistry, 26:1152), GC and CG base pairs “buckle” by approximately 9° and 15° respectively to prevent excessive van der Waals contacts. Also, the base pairs separate from a nominal distance of 3.4 Å to 6.8 Å when accommodating the drug, and these distortions lead to a total DNA unwinding angle of approximately 8° (5.2 measured from solution studies; DiMarco and Arcamone, 1975, Arzneim-Forsch. (Drug Res.), 25:368) and a distortion of the tertiary structure of the helix, although it is still closer to the B-DNA conformation. Several factors play a role in the stabilization of the drug-DNA complex. Anthracycline is stabilized by electrostatic hydrogen bond and stacking p-bond interactions between the electron-deficient quinone-based chromophore and the electron-rich purine-pyrimidine bases. Hydrogen bonds are involved in the stabilization of the complex, assisted by way of several water molecules and a solvated sodium cation. Also, the hydrogen atom of the charged amino group is hydrogen bonded to 0-2 of the thiamine base (T10) and two water molecules.

In some embodiments, the present invention provides an intercalating moiety which does not have therapeutic or diagnostic properties by itself, but is associated with a therapeutic or diagnostic agent to be delivered. In some embodiments, the association is covalent. In some embodiments, the association is non-covalent. In some embodiments, the association is mediated by a linker (e.g. a cleavable linker). To give but one example, an aromatic ring structure which does not have therapeutic or diagnostic properties may be associated with a therapeutic or diagnostic agent. The ring structure may be able to intercalate between the base pairs of an aptamer or spiegelmer for targeted delivery of the agent.

Nucleic Acid Targeting Moieties

According to the present invention, the inventive complexes comprise one or more nucleic acid targeting moieties associated with one or more intercalating agents. In general, a nucleic acid targeting moiety is any polynucleotide that binds to a component associated with an organ, tissue, cell, extracellular matrix component, and/or intracellular compartment. In some embodiments, such a component is referred to as a “target” or a “marker,” and these are discussed in further detail below.

In some embodiments, nucleic acid targeting moieties bind to an organ, tissue, cell, extracellular matrix component, and/or intracellular compartment that is associated with a specific developmental stage or a specific disease state. In some embodiments, a target is an antigen on the surface of a cell, such as a cell surface receptor, an integrin, a transmembrane protein, an ion channel, a membrane transport protein, a glycoprotein, a carbohydrate, and the like. In some embodiments, a target is an intracellular protein. In some embodiments, a target is a soluble protein, such as immunoglobulin. In certain specific embodiments, a target is a tumor marker. In some embodiments, a tumor marker is an antigen that is present in a tumor that is not present in normal organs, tissues, and/or cells. In some embodiments, a tumor marker is an antigen that is more prevalent in a tumor than in normal organs, tissues, and/or cells. In some embodiments, a tumor marker is an antigen that is more prevalent in malignant cancer cells than in normal cells.

In some embodiments, a target is preferentially expressed in tumor tissues and/or cells versus normal tissues and/or cells. To give but one example, when compared with expression in normal tissues, expression of prostate specific membrane antigen (PSMA) is at least 10-fold overexpressed in malignant prostate relative to normal tissue, and the level of PSMA expression is further up-regulated as the disease progresses into metastatic phases (Silver et al., 1997, Clin. Cancer Res., 3:81).

In some embodiments, nucleic acid targeting moieties are aptamers. An aptamer is typically a polynucleotide that binds to a specific target structure that is associated with a particular organ, tissue, cell, extracellular matrix component, and/or intracellular compartment. In general, the targeting function of the aptamer is based on the three-dimensional structure of the aptamer. In some embodiments, binding of an aptamer to a target is typically mediated by the interaction between the two- and/or three-dimensional structures of both the aptamer and the target. In some embodiments, binding of an aptamer to a target is not solely based on the primary sequence of the aptamer, but depends on the three-dimensional structure(s) of the aptamer and/or target. In some embodiments, aptamers bind to their targets via complementary Watson-Crick base pairing which is interrupted by structures (e.g. hairpin loops) that disrupt base pairing.

In some embodiments, nucleic acid targeting moieties are spiegelmers (PCT Publications WO 98/08856, WO 02/100442, and WO 06/117217). In general, spiegelmers are synthetic, mirror-image nucleic acids that can specifically bind to a target (i.e. mirror image aptamers). Spiegelmers are characterized by structural features which make them not susceptible to exo- and endo-nucleases.

One of ordinary skill in the art will recognize that any nucleic acid targeting moiety (e.g. aptamer or spiegelmer) that is capable of specifically binding to a target can be used in accordance with the present invention. In some embodiments, nucleic acid targeting moieties to be used in accordance with the present invention may target a marker associated with a disease, disorder, and/or condition. In some embodiments, nucleic acid targeting moieties to be used in accordance with the present invention may target cancer-associated targets. In some embodiments, nucleic acid targeting moieties to be used in accordance with the present invention may target tumor markers. Any type of cancer and/or any tumor marker may be targeted using nucleic acid targeting moieties in accordance with the present invention. To give but a few examples, nucleic acid targeting moieties may target markers associated with prostate cancer, lung cancer, breast cancer, colorectal cancer, bladder cancer, pancreatic cancer, endometrial cancer, ovarian cancer, bone cancer, esophageal cancer, liver cancer, stomach cancer, brain tumors, cutaneous melanoma, and/or leukemia.

In certain embodiments, aptamers or spiegelmers to be used in accordance with the present invention may target prostate cancer associated antigens, such as PSMA. Exemplary PSMA-targeting aptamers to be used in accordance with the present invention include, but are not limited to, the A10 aptamer, having a nucleotide sequence of 5′-GGGAGGACGAUGCGGAUCAGCCAUGUUUACGUCACUCCUUGUCAAUCCUCAUC GGCAGACGACUCGCCCGA-3′ (SEQ ID NO.: 1) (Lupold et al., 2002, Cancer Res., 62:4029), the A9 aptamer, having nucleotide sequence of 5′-GGGAGGACGAUGCGGACCGAAAAAGACCUGACUUCUAUACUAAGUCUACGUUC CCAGACGACUCGCCCGA-3′ (SEQ ID NO.: 2) (Lupold et al., 2002, Cancer Res., 62:4029; and Chu et al., 2006, Nuc. Acid Res., 34:e73), derivatives thereof, and/or characteristic portions thereof.

In some embodiments, a nucleotide sequence that is homologous to a nucleic acid nucleic acid targeting moiety may be used in accordance with the present invention. In some embodiments, a nucleotide sequence is considered to be “homologous” to a nucleic acid nucleic acid targeting moiety if it comprises fewer than 30, 25, 20, 15, 10, 5, 4, 3, 2, or 1 nucleic acid substitutions relative to the aptamer or spiegelmer. In some embodiments, a nucleotide sequence is considered to be “homologous” to a nucleic acid nucleic acid targeting moiety if their sequences are at least 25%, 30%, 35%, 40%, 45%, 50%, 55%, 60%, 65%, 70%, 75%, 80%, 85%, 90%, 95%, or 99% identical. In some embodiments, a nucleic acid sequence is considered to be “homologous” to a nucleic acid nucleic acid targeting moiety if their sequences are at least 25%, 30%, 35%, 40%, 45%, 50%, 55%, 60%, 65%, 70%, 75%, 80%, 85%, 90%, 95%, or 99% similar.

Nucleic acids of the present invention (including nucleic acid nucleic acid targeting moieties and/or functional RNAs to be delivered, e.g., RNAi-inducing entities, ribozymes, tRNAs, etc., described in further detail below) may be prepared according to any available technique including, but not limited to chemical synthesis, enzymatic synthesis, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in molecular biology, v. 288 (Clifton, N.J.) Totowa, N.J.: Humana Press, 2005).

The nucleic acid that forms the nucleic acid nucleic acid targeting moiety may comprise naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid nucleic acid targeting moiety can be replaced with a hydrocarbon linker or a polyether linker provided that the binding affinity and selectivity of the nucleic acid nucleic acid targeting moiety is not substantially reduced by the substitution (e.g., the dissociation constant of the nucleic acid nucleic acid targeting moiety for the target should not be greater than about 1×10⁻³ M).

It will be appreciated by those of ordinary skill in the art that nucleic acids in accordance with the present invention may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein disclose a wide variety of specific nucleotide analogs and modifications that may be used. See Crooke, S. (ed.) Antisense Drug Technology: Principles, Strategies, and Applications (1^{st} ed), Marcel Dekker; ISBN: 0824705661; 1st edition (2001) and references therein. For example, 2′-modifications include halo, alkoxy and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids comprising a variety of different nucleotide analogs, modified backbones, or non-naturally occurring internucleoside linkages can be utilized in accordance with the present invention. Nucleic acids of the present invention may include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially affected. To give but one example, modifications may be located at any position of a nucleic acid targeting moiety such that the ability of the nucleic acid targeting moiety to specifically bind to the target is not substantially affected. The modified region may be at the 5′-end and/or the 3′-end of one or both strands. For example, modified nucleic acid targeting moieties in which approximately 1-5 residues at the 5′ and/or 3′ end of either of both strands are nucleotide analogs and/or have a backbone modification have been employed. The modification may be a 5′ or 3′ terminal modification. One or both nucleic acid strands may comprise at least 50% unmodified nucleotides, at least 80% unmodified nucleotides, at least 90% unmodified nucleotides, or 100% unmodified nucleotides.

Nucleic acids in accordance with the present invention may, for example, comprise a modification to a sugar, nucleoside, or internucleoside linkage such as those described in U.S. Patent Application Publications 2003/0175950, 2004/0192626, 2004/0092470, 2005/0020525, and 2005/0032733. The present invention encompasses the use of any nucleic acid having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g., using any appropriate assay known in the art, for example, to select those that result in improved delivery of a therapeutic or diagnostic agent, improved specific binding of an nucleic acid targeting moiety to a target, etc. In some embodiments, nucleic acids in accordance with the present invention may comprise one or more non-natural nucleoside linkages. In some embodiments, one or more internal nucleotides at the 3′-end, 5′-end, or both 3′- and 5′-ends of the nucleic acid targeting moiety are inverted to yield a linkage such as a 3′-3′ linkage or a 5′-5′ linkage.

In some embodiments, nucleic acids in accordance with the present invention are not synthetic, but are naturally-occurring entities that have been isolated from their natural environments.

Any method can be used to design novel nucleic acid targeting moieties (see, e.g., U.S. Pat. Nos. 6,716,583; 6,465,189; 6,482,594; 6,458,543; 6,458,539; 6,376,190; 6,344,318; 6,242,246; 6,184,364; 6,001,577; 5,958,691; 5,874,218; 5,853,984; 5,843,732; 5,843,653; 5,817,785; 5,789,163; 5,763,177; 5,696,249; 5,660,985; 5,595,877; 5,567,588; and 5,270,163; and U.S. Patent Application Publications 2005/0069910, 2004/0072234, 2004/0043923, 2003/0087301, 2003/0054360, and 2002/0064780). The present invention provides methods for designing novel nucleic acid targeting moieties. The present invention further provides methods for isolating or identifying novel nucleic acid targeting moieties from a mixture of candidate nucleic acid targeting moieties.

Nucleic acid targeting moieties that bind to a protein, a carbohydrate, a lipid, and/or a nucleic acid can be designed and/or identified. In some embodiments, nucleic acid targeting moieties can be designed and/or identified for use in the complexes of the invention that bind to proteins and/or characteristic portions thereof, such as tumor-markers, integrins, cell surface receptors, transmembrane proteins, intercellular proteins, ion channels, membrane transporter proteins, enzymes, antibodies, chimeric proteins etc. In some embodiments, nucleic acid targeting moieties can be designed and/or identified for use in the complexes of the invention that bind to carbohydrates and/or characteristic portions thereof, such as glycoproteins, sugars (e.g., monosaccharides, disaccharides and polysaccharides), glycocalyx (i.e., the carbohydrate-rich peripheral zone on the outside surface of most eukaryotic cells) etc. In some embodiments, nucleic acid targeting moieties can be designed and/or identified for use in the complexes of the invention that bind to lipids and/or characteristic portions thereof, such as oils, saturated fatty acids, unsaturated fatty acids, glycerides, hormones, steroids (e.g., cholesterol, bile acids), vitamins (e.g. vitamin E), phospholipids, sphingolipids, lipoproteins etc. In some embodiments, nucleic acid targeting moieties can be designed and/or identified for use in the complexes of the invention that bind to nucleic acids and/or characteristic portions thereof, such as DNA nucleic acids; RNA nucleic acids; modified DNA nucleic acids; modified RNA nucleic acids; and nucleic acids that include any combination of DNA, RNA, modified DNA, and modified RNA; etc.

Nucleic acid targeting moieties (e.g. aptamers or spiegelmers) may be designed and/or identified using any available method. In some embodiments, nucleic acid targeting moieties are designed and/or identified by identifying nucleic acid targeting moieties from a candidate mixture of nucleic acids. Systemic Evolution of Ligands by Exponential Enrichment (SELEX), or a variation thereof, is a commonly used method of identifying nucleic acid targeting moieties that bind to a target from a candidate mixture of nucleic acids.

The SELEX process for designing and/or identifying nucleic acid targeting moieties is described in U.S. Pat. Nos. 6,482,594; 6,458,543; 6,458,539; 6,376,190; 6,344,318; 6,242,246; 6,184,364; 6,001,577; 5,958,691; 5,874,218; 5,853,984; 5,843,732; 5,843,653; 5,817,785; 5,789,163; 5,763,177; 5,696,249; 5,660,985; 5,595,877; 5,567,588; and 5,270,163. Briefly, the basic SELEX process may be defined by the following series of steps:

1) A candidate mixture of nucleic acids of differing sequence is prepared. A candidate mixture generally includes regions of fixed sequences (i.e., each of the members of the candidate mixture contains the same sequences in the same location) and regions of randomized sequences. Fixed sequence regions are selected to assist in the amplification steps described below; to mimic a sequence known to bind to the target; and/or to enhance the potential of a given structural arrangement of the nucleic acids in the candidate mixture. Randomized sequences can be totally randomized (i.e., the probability of finding a base at any position being one in four) or only partially randomized (i.e., the probability of finding a base at any location can be selected at any level between 0% and 100%).

2) The candidate mixture is contacted with a selected target under conditions favorable for binding between the target and members of the candidate mixture. Under these circumstances, the interaction between the target and the nucleic acids of the candidate mixture can be considered as forming nucleic acid-target pairs between the target and the nucleic acids having the strongest affinity for the target.

3) Nucleic acids with the highest affinity for the target are partitioned from those nucleic acids with lesser affinity to the target. Because only an extremely small number of sequences (and possibly only one molecule of nucleic acid) corresponding to the highest affinity nucleic acid targeting moieties exist in the candidate mixture, it is generally desirable to set the partitioning criteria so that a significant amount of the nucleic acid targeting moieties in the candidate mixture (approximately 0.1%-10%) is retained during partitioning.

4) Those nucleic acid targeting moieties selected during partitioning as having the relatively higher affinity to the target are then amplified to create a new candidate mixture that is enriched in nucleic acid targeting moieties having a relatively higher affinity for the target.

5) By repeating the partitioning and amplifying steps above, the newly formed candidate mixture contains fewer and fewer unique sequences, and the average degree of affinity of the nucleic acid mixture to the target will generally increase. Taken to its extreme, the SELEX process will yield a candidate mixture containing one or a small number of unique nucleic acid targeting moieties representing those nucleic acid targeting moieties from the original candidate mixture having the highest affinity to the target. In general, nucleic acid targeting moieties identified will have dissociation constants with the target of about 1×10⁻⁶ M or less. Typically, the dissociation constant of the nucleic acid targeting moiety and the target will be in the range of between about 1×10⁻⁸ M and about 1×10⁻¹² M.

Nucleic acid targeting moieties that bind selectively to any target can be isolated by the SELEX process, or a variation thereof, provided that the target can be used as a target in the SELEX process.

Alternatively or additionally, Polyplex In Vivo Combinatorial Optimization (PICO) is a method that can be used to identify nucleic acid targeting moieties (e.g. aptamers or spiegelmers) that bind to a target from a candidate mixture of nucleic acids in vivo and/or in vitro and is described in co-pending PCT Application US06/47975, entitled “System for Screening Particles,” filed Dec. 15, 2006. Briefly, the basic PICO process may be defined by the following series of steps:

1) A library comprising a plurality of nucleic acids is provided and associated with particles (e.g. nanoparticles), thereby producing targeted particles.

2) The targeted particles are administered to an animal (e.g. mouse) under conditions in which the particles can migrate to a tissue of interest (e.g. tumor).

3) A first population of targeted particles that have migrated to the cells, tissue, or organ of interest is recovered. The nucleic acid targeting moieties associated with the first population of targeted particles are amplified and associated with new particles.

4) Selection is repeated several times to yield a set of nucleic acid targeting moieties with specificity for the target tissue that is increased relative to the original library.

Nucleic acid targeting moieties that bind selectively to any in vivo and/or in vitro target can be isolated by the PICO process, provided that the target can be used as a target in the PICO process.

Spiegelmers can be designed based on the same techniques that are used for designing aptamers, utilizing the concept that if an aptamer binds its natural target, the mirror image of the aptamer will identically bind the mirror image of the natural target. For example, if the process of aptamer selection is carried out against the mirror image of the aptamer target, an aptamer against this unnatural mirror image is obtained. The corresponding mirror image nucleic acid (L-oligonucleotide) of this aptamer (i.e. the Spiegelmer) can bind to the natural target ligand with similar binding characteristics as the aptamer itself.

Targets

In certain embodiments, complexes in accordance with the present invention comprise a nucleic acid targeting moiety which specifically binds to one or more targets (e.g. antigens) associated with an organ, tissue, cell, extracellular matrix component, and/or intracellular compartment. In some embodiments, complexes comprise a nucleic acid targeting moiety which specifically binds to targets associated with a particular organ or organ system. In some embodiments, complexes in accordance with the present invention comprise a nucleic acid targeting moiety which specifically binds to one or more intracellular targets (e.g. organelle, intracellular protein). In some embodiments, complexes comprise a nucleic acid targeting moiety which specifically binds to targets associated with diseased organs, tissues, cells, extracellular matrix components, and/or intracellular compartments. In some embodiments, complexes comprise a nucleic acid targeting moiety which specifically binds to targets associated with particular cell types (e.g. endothelial cells, cancer cells, malignant cells, prostate cancer cells, etc.).

In some embodiments, complexes in accordance with the present invention comprise a nucleic acid targeting moiety which binds to a target that is specific for one or more particular tissue types (e.g. liver tissue vs. prostate tissue). In some embodiments, complexes in accordance with the present invention comprise a nucleic acid targeting moiety which binds to a target that is specific for one or more particular cell types (e.g. T cells vs. B cells). In some embodiments, complexes in accordance with the present invention comprise a nucleic acid targeting moiety which binds to a target that is specific for one or more particular disease states (e.g. tumor cells vs. healthy cells). In some embodiments, complexes in accordance with the present invention comprise a nucleic acid targeting moiety which binds to a target that is specific for one or more particular developmental stages (e.g. stem cells vs. differentiated cells).

In some embodiments, a target may be a marker that is exclusively or primarily associated with one or a few cell types, with one or a few diseases, and/or with one or a few developmental stages. A cell type specific marker is typically expressed at levels at least 2 fold greater in that cell type than in a reference population of cells which may consist, for example, of a mixture containing cells from a plurality (e.g., 5-10 or more) of different tissues or organs in approximately equal amounts. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 1000 fold, or at least 1000 fold greater than its average expression in a reference population. Detection or measurement of a cell type specific marker may make it possible to distinguish the cell type or types of interest from cells of many, most, or all other types.

In some embodiments, a target can comprise a protein, a carbohydrate, a lipid, and/or a nucleic acid. In certain embodiments, a target can comprise a protein and/or characteristic portion thereof, such as a tumor-marker, integrin, cell surface receptor, transmembrane protein, intercellular protein, ion channel, membrane transporter protein, enzyme, antibody, chimeric protein, glycoprotein, etc. In certain embodiments, a target can comprise a carbohydrate and/or characteristic portion thereof, such as a glycoprotein, sugar (e.g., monosaccharide, disaccharide, polysaccharide), glycocalyx (i.e., the carbohydrate-rich peripheral zone on the outside surface of most eukaryotic cells) etc. In certain embodiments, a target can comprise a lipid and/or characteristic portion thereof, such as an oil, fatty acid, glyceride, hormone, steroid (e.g., cholesterol, bile acid), vitamin (e.g. vitamin E), phospholipid, sphingolipid, lipoprotein, etc. In certain embodiments, a target can comprise a nucleic acid and/or characteristic portion thereof, such as a DNA nucleic acid; RNA nucleic acid; modified DNA nucleic acid; modified RNA nucleic acid; nucleic acid that includes any combination of DNA, RNA, modified DNA, and modified RNA; etc.

Numerous markers are known in the art. Typical markers include cell surface proteins, e.g., receptors. Exemplary receptors include, but are not limited to, the transferrin receptor; LDL receptor; growth factor receptors such as epidermal growth factor receptor family members (e.g., EGFR, HER-2, HER-3, HER-4, HER-2/neu) or vascular endothelial growth factor receptors; cytokine receptors; cell adhesion molecules; integrins; selectins; CD molecules; etc. The marker can be a molecule that is present exclusively or in higher amounts on a malignant cell, e.g., a tumor antigen. For example, prostate-specific membrane antigen (PSMA) is expressed at the surface of prostate cancer cells. In certain embodiments of the invention the marker is an endothelial cell marker.

In certain embodiments of the invention a marker is a tumor marker. The marker may be a polypeptide that is expressed at higher levels on dividing than on non-dividing cells. For example, Her-2/neu (also known as ErbB-2) is a member of the EGF receptor family and is expressed on the cell surface of tumors associated with breast cancer. To give another example, a peptide known as F3 is a suitable targeting agent for directing a nanoparticle to nucleolin (Porkka et al., 2002, Proc. Natl. Acad. Sci., USA, 99:7444; and Christian et al., 2003, J. Cell Biol., 163:871). As described in the Examples, targeted particles comprising a nanoparticle and the A10 aptamer (which specifically binds to PSMA) were able to specifically and effectively deliver docetaxel to prostate cancer tumors.

In some embodiments, a marker is a prostate cancer marker. In some embodiments, a prostate cancer marker is expressed by prostate cells but not by other cell types. In some embodiments, a prostate cancer marker is expressed by prostate cancer tumor cells but not by other cell types. Any prostate cancer marker can be used in accordance with the present invention. To give but one non-limiting example, in certain embodiments, a prostate cancer marker is prostate specific membrane antigen (PSMA), a 100 kDa transmembrane glycoprotein that is expressed in most prostatic tissues, but is more highly expressed in prostatic cancer tissue than in normal tissue.

In some embodiments, a prostate cancer marker is transmembrane protein 24P4C12 (U.S. Patent Application Publication 2005/0019870). In some embodiments, a prostate cancer marker is prostate stem cell antigen (U.S. Patent Application Publication 2006/0269557). In some embodiments, a prostate cancer marker is the androgen receptor (see, e.g., U.S. Pat. Nos. 7,026,500; 7,022,870; 6,998,500; 6,995,284; 6,838,484; 6,569,896; 6,492,554; and U.S. Patent Application Publications 2006/0287547; 2006/0276540; 2006/0258628; 2006/0241180; 2006/0183931; 2006/0035966; 2006/0009529; 2006/0004042; 2005/0033074; 2004/0260108; 2004/0260092; 2004/0167103; 2004/0147550; 2004/0147489; 2004/0087810; 2004/0067979; 2004/0052727; 2004/0029913; 2004/0014975; 2003/0232792; 2003/0232013; 2003/0225040; 2003/0162761; 2004/0087810; 2003/0022868; 2002/0173495; 2002/0099096; and 2002/0099036). In some embodiments, a prostate cancer marker is calveolin (U.S. Pat. No. 7,029,859; and U.S. Patent Application Publications 2003/0003103 and 2001/0012890). In some embodiments, a prostate cancer marker is prostate specific antigen. In some embodiments, a prostate cancer marker is human glandular kallikrein 2. In some embodiments, a prostate cancer marker is prostatic acid phosphatase. In some embodiments, a prostate cancer marker is insulin-like growth factor and/or insulin-like growth factor binding protein. In some embodiments, a prostate cancer marker is PHOR-1 (U.S. Patent Application Publication 2004/0248088). In some embodiments, a prostate cancer marker is C-type lectin transmembrane antigen (U.S. Patent Application Publication 2005/0019872). In some embodiments, a prostate cancer marker is a protein encoded by 103P2D6 (U.S. Patent Application Publication 2003/0219766). In some embodiments, a prostate cancer marker is a prostatic specific reductase polypeptide (U.S. Pat. No. 5,786,204; and U.S. Patent Application Publication 2002/0150578). In some embodiments, a prostate cancer marker is an IL-11 receptor-α (U.S. Patent Application Publication 2005/0191294).

Particles Associated with Inventive Complexes

In general, targeted particles of the present invention comprise any type of particle. Any particle can be used in accordance with the present invention. In some embodiments, particles are biodegradable and biocompatible. In general, a biocompatible substance is not toxic to cells. In some embodiments, a substance is considered to be biocompatible if its addition to cells results in less than a certain threshold of cell death. In some embodiments, a substance is considered to be biocompatible if its addition to cells does not induce adverse effects. In general, a biodegradable substance is one that undergoes breakdown under physiological conditions over the course of a therapeutically relevant time period (e.g., weeks, months, or years). In some embodiments, a biodegradable substance is a substance that can be broken down by cellular machinery. In some embodiments, a biodegradable substance is a substance that can be broken down by chemical processes. In some embodiments, a particle is a substance that is both biocompatible and biodegradable. In some embodiments, a particle is a substance that is biocompatible, but not biodegradable. In some embodiments, a particle is a substance that is biodegradable, but not biocompatible.

In some embodiments, a particle which is biocompatible and/or biodegradable may be associated with a therapeutic or diagnostic agent that is not biocompatible, is not biodegradable, or is neither biocompatible nor biodegradable (e.g. a cytotoxic agent). In some embodiments, a particle which is biocompatible and/or biodegradable may be associated with a therapeutic or diagnostic agent that is also biocompatible and/or biodegradable.

In general, a particle in accordance with the present invention is any entity having a greatest dimension (e.g. diameter) of less than 100 microns (μm). In some embodiments, inventive particles have a greatest dimension of less than 10 μm. In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 300 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

In some embodiments, particles have a diameter of approximately 1000 nm. In some embodiments, particles have a diameter of approximately 750 nm. In some embodiments, particles have a diameter of approximately 500 nm. In some embodiments, particles have a diameter of approximately 450 nm. In some embodiments, particles have a diameter of approximately 400 nm. In some embodiments, particles have a diameter of approximately 350 nm. In some embodiments, particles have a diameter of approximately 300 nm. In some embodiments, particles have a diameter of approximately 275 nm. In some embodiments, particles have a diameter of approximately 250 nm. In some embodiments, particles have a diameter of approximately 225 nm. In some embodiments, particles have a diameter of approximately 200 nm. In some embodiments, particles have a diameter of approximately 175 nm. In some embodiments, particles have a diameter of approximately 150 nm. In some embodiments, particles have a diameter of approximately 125 nm. In some embodiments, particles have a diameter of approximately 100 nm. In some embodiments, particles have a diameter of approximately 75 nm. In some embodiments, particles have a diameter of approximately 50 nm. In some embodiments, particles have a diameter of approximately 25 nm.

In certain embodiments, particles are greater in size than the renal excretion limit (e.g. particles having diameters of greater than 6 nm). In certain embodiments, particles are small enough to avoid clearance of particles from the bloodstream by the liver (e.g. particles having diameters of less than 1000 nm). In general, physiochemical features of particles should allow a targeted particle to circulate longer in plasma by decreasing renal excretion and liver clearance.

It is often desirable to use a population of particles that is relatively uniform in terms of size, shape, and/or composition so that each particle has similar properties. For example, at least 80%, at least 90%, or at least 95% of the particles may have a diameter or greatest dimension that falls within 5%, 10%, or 20% of the average diameter or greatest dimension. In some embodiments, a population of particles may be heterogeneous with respect to size, shape, and/or composition.

Zeta potential is a measurement of surface potential of a particle. In some embodiments, particles have a zeta potential ranging between −50 mV and +50 mV. In some embodiments, particles have a zeta potential ranging between −25 mV and +25 mV. In some embodiments, particles have a zeta potential ranging between −10 mV and +10 mV. In some embodiments, particles have a zeta potential ranging between −5 mV and +5 mV. In some embodiments, particles have a zeta potential ranging between 0 mV and +50 mV. In some embodiments, particles have a zeta potential ranging between 0 mV and +25 mV. In some embodiments, particles have a zeta potential ranging between 0 mV and +10 mV. In some embodiments, particles have a zeta potential ranging between 0 mV and +5 mV. In some embodiments, particles have a zeta potential ranging between −50 mV and 0 mV. In some embodiments, particles have a zeta potential ranging between −25 mV and 0 mV. In some embodiments, particles have a zeta potential ranging between −10 mV and 0 mV. In some embodiments, particles have a zeta potential ranging between −5 mV and 0 mV. In some embodiments, particles have a substantially neutral zeta potential (i.e. approximately 0 mV).

A variety of different particles can be used in accordance with the present invention. In some embodiments, particles are spheres or spheroids. In some embodiments, particles are spheres or spheroids. In some embodiments, particles are flat or plate-shaped. In some embodiments, particles are cubes or cuboids. In some embodiments, particles are ovals or ellipses. In some embodiments, particles are cylinders, cones, or pyramids.

In some embodiments, particles are microparticles (e.g. microspheres). In general, a “microparticle” refers to any particle having a diameter of less than 1000 μm. In some embodiments, particles are nanoparticles (e.g. nanospheres). In general, a “nanoparticle” refers to any particle having a diameter of less than 1000 nm. In some embodiments, particles are picoparticles (e.g. picospheres). In general, a “picoparticle” refers to any particle having a diameter of less than 1 nm. In some embodiments, particles are liposomes. In some embodiments, particles are micelles.

Particles can be solid or hollow and can comprise one or more layers (e.g., nanoshells, nanorings). In some embodiments, each layer has a unique composition and unique properties relative to the other layer(s). To give but one example, particles may have a core/shell structure, wherein the core is one layer and the shell is a second layer. Particles may comprise a plurality of different layers. In some embodiments, one layer may be substantially cross-linked, a second layer is not substantially cross-linked, and so forth. In some embodiments, one, a few, or all of the different layers may comprise one or more therapeutic or diagnostic agents to be delivered. In some embodiments, one layer comprises an agent to be delivered, a second layer does not comprise an agent to be delivered, and so forth. In some embodiments, each individual layer comprises a different agent or set of agents to be delivered.

In certain embodiments of the invention, a particle is porous, by which is meant that the particle contains holes or channels, which are typically small compared with the size of a particle. For example a particle may be a porous silica particle, e.g., a mesoporous silica nanoparticle or may have a coating of mesoporous silica (Lin et al., 2005, J. Am. Chem. Soc., 17:4570). Particles may have pores ranging from about 1 nm to about 50 nm in diameter, e.g., between about 1 and 20 nm in diameter. Between about 10% and 95% of the volume of a particle may consist of voids within the pores or channels.

Particles may have a coating layer. Use of a biocompatible coating layer can be advantageous, e.g., if the particles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, other nanoparticles that can be associated with inventive nanoparticles etc. Coatings may be applied or assembled in a variety of ways such as by dipping, using a layer-by-layer technique, by self-assembly, conjugation, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

In some embodiments, particles may optionally comprise one or more dispersion media, surfactants, release-retarding ingredients, or other pharmaceutically acceptable excipient. In some embodiments, particles may optionally comprise one or more plasticizers or additives.

Particles Comprising a Polymeric Matrix

In some embodiments, particles can comprise a matrix of polymers. In some embodiments, a therapeutic or diagnostic agent and/or nucleic acid targeting moiety can be covalently associated with the surface of a polymeric matrix. In some embodiments, covalent association is mediated by a linker. In some embodiments, a therapeutic or diagnostic agent and/or nucleic acid targeting moiety can be non-covalently associated with the surface of a polymeric matrix. In some embodiments, a therapeutic or diagnostic agent and/or nucleic acid targeting moiety can be associated with the surface of, encapsulated within, surrounded by, and/or dispersed throughout a polymeric matrix.

A wide variety of polymers and methods for forming particles therefrom are known in the art of drug delivery. In some embodiments of the invention, the matrix of a particle comprises one or more polymers. Any polymer may be used in accordance with the present invention. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers include polyalkylenes (e.g. polyethylenes), polycarbonates (e.g. poly(1,3-dioxan-2one)), polyanhydrides (e.g. poly(sebacic anhydride)), polyhydroxyacids (e.g. poly(β-hydroxyalkanoate)), polyfumarates, polycaprolactones, polyamides (e.g. polycaprolactam), polyacetals, polyethers, polyesters (e.g. polylactide, polyglycolide), poly(orthoesters), polyvinyl alcohols, polyurethanes, polyphosphazenes, polyacrylates, polymethacrylates, polycyanoacrylates, polyureas, polystyrenes, and polyamines. In some embodiments, polymers in accordance with the present invention include polymers which have been approved for use in humans by the U.S. Food and Drug Administration (FDA) under 21 C.F.R. §177.2600, including but not limited to polyesters (e.g. polylactic acid, polyglycolic acid, poly(lactic-co-glycolic acid), polycaprolactone, polyvalerolactone, poly(1,3-dioxan-2one)); polyanhydrides (e.g. poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g. phosphate group, sulphate group, carboxylate group); cationic groups (e.g. quaternary amine group); or polar groups (e.g. hydroxyl group, thiol group, amine group).

In some embodiments, polymers may be modified with one or more moieties and/or functional groups. Any moiety or functional group can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301).

In some embodiments, may be modified with a lipid or fatty acid group, properties of which are described in further detail below. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g. PEGylated PLA, PEGylated PGA, PEGylated PLGA, and derivatives thereof. In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 65:35, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, RNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633). Poly(4-hydroxy-L-proline ester) was recently demonstrated to condense plasmid DNA through electrostatic interactions, and to mediate gene transfer (Putnam et al., 1999, Macromolecules, 32:3658; and Lim et al., 1999, J. Am. Chem. Soc., 121:5633). These new polymers are less toxic than poly(lysine) and PEI, and they degrade into non-toxic metabolites.

In some embodiments, polymers can be anionic polymers. In some embodiments, anionic polymers comprise carboxyl, sulfate, or groups. To give but a few examples, anionic polymers include, but are not limited to, dextran sulfate, heparan sulfate, alginic acid, polyvinylcarboxylic acid, arabic acid carboxymethylcellulose, and the like. In some embodiments, anionic polymers are provided as a salt (e.g. sodium salt).

In some embodiments, a polymer in accordance with the present invention may be a carbohydrate, properties of which are described in further detail below. In some embodiments, a carbohydrate may be a polysaccharide comprising simple sugars (or their derivatives) connected by glycosidic bonds, as known in the art. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan.

In some embodiments, a polymer in accordance with the present invention may be a protein or peptide, properties of which are described in further detail below. Exemplary proteins that may be used in accordance with the present invention include, but are not limited to, albumin, collagen, a poly(amino acid) (e.g. polylysine), an antibody, etc.

In some embodiments, a polymer in accordance with the present invention may be a nucleic acid (i.e. polynucleotide), properties of which are described in further detail below. Exemplary polynucleotides that may be used in accordance with the present invention include, but are not limited to, DNA, RNA, etc.

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step.

It is further to be understood that inventive targeted particles may comprise block copolymers, graft copolymers, blends, mixtures, and/or adducts of any of the foregoing and other polymers.

Those skilled in the art will recognize that the polymers listed herein represent an exemplary, not comprehensive, list of polymers that can be of use in accordance with the present invention.

Non-Polymeric Particles

In some embodiments, particles can be non-polymeric particles (e.g. metal particles, quantum dots, ceramic particles, polymers comprising inorganic materials, bone-derived materials, bone substitutes, viral particles, etc.). In some embodiments, a therapeutic or diagnostic agent to be delivered can be associated with the surface of such a non-polymeric particle. In some embodiments, a non-polymeric particle is an aggregate of non-polymeric components, such as an aggregate of metal atoms (e.g. gold atoms). In some embodiments, a therapeutic or diagnostic agent to be delivered can be associated with the surface of and/or encapsulated within, surrounded by, and/or dispersed throughout an aggregate of non-polymeric components.

In certain embodiments of the invention, non-polymeric particles comprise gradient or homogeneous alloys. In certain embodiments of the invention, particles are composite particles made of two or more materials, of which one, more than one, or all of the materials possess an optically or magnetically detectable property, as discussed in further detail below.

In certain embodiments of the invention, particles comprise silica (SiO₂). For example, a particle may consist at least in part of silica, e.g., it may consist essentially of silica or may have an optional coating layer composed of a different material. In some embodiments, a particle has a silica core and an outside layer composed of one or more other materials. In some embodiments, a particle has an outer layer of silica and a core composed of one or more other materials. The amount of silica in the particle, or in a core or coating layer comprising silica, can range from approximately 5% to 100% by mass, volume, or number of atoms, or can assume any value or range between 5% and 100%.

Preparation of Particles

Particles (e.g. nanoparticles, microparticles) may be prepared using any method known in the art. For example, particulate formulations can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanoparticles have been described (Pellegrino et al., 2005, Small, 1:48; Murray et al., 2000, Ann. Rev. Mat. Sci., 30:545; and Trindade et al., 2001, Chem. Mat., 13:3843).

In certain embodiments, particles are prepared by the nanoprecipitation process or spray drying. Conditions used in preparing particles may be altered to yield particles of a desired size or property (e.g., hydrophobicity, hydrophilicity, external morphology, “stickiness,” shape, etc.). The method of preparing the particle and the conditions (e.g., solvent, temperature, concentration, air flow rate, etc.) used may depend on the therapeutic or diagnostic agent to be delivered and/or the composition of the polymer matrix.

Methods for making microparticles for delivery of encapsulated agents are described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, δ: 275; and Mathiowitz et al., 1988, J. Appl. Polymer Sci., 35:755).

If particles prepared by any of the above methods have a size range outside of the desired range, particles can be sized, for example, using a sieve.

Surfactants

In some embodiments, particles may optionally comprise one or more surfactants. In some embodiments, a surfactant can promote the production of particles with increased stability, improved uniformity, or increased viscosity. Surfactants can be particularly useful in embodiments that utilize two or more dispersion media. The percent of surfactant in particles can range from 0% to 99% by weight, from 10% to 99% by weight, from 25% to 99% by weight, from 50% to 99% by weight, or from 75% to 99% by weight. In some embodiments, the percent of surfactant in particles can range from 0% to 75% by weight, from 0% to 50% by weight, from 0% to 25% by weight, or from 0% to 10% by weight. In some embodiments, the percent of surfactant in particles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

Any surfactant known in the art is suitable for use in making particles in accordance with the present invention. Such surfactants include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span 85) glycocholate; sorbitan monolaurate (Span 20); polysorbate 20 (Tween-20); polysorbate 60 (Tween-60); polysorbate 65 (Tween-65); polysorbate 80 (Tween-80); polysorbate 85 (Tween-85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. The surfactant component may be a mixture of different surfactants. These surfactants may be extracted and purified from a natural source or may be prepared synthetically in a laboratory. In certain specific embodiments, surfactants are commercially available.

Those skilled in the art will recognize that this is an exemplary, not comprehensive, list of substances with surfactant activity. Any surfactant may be used in the production of particles to be used in accordance with the present invention.

Lipids

In some embodiments, particles may optionally comprise one or more lipids. The percent of lipid in particles can range from 0% to 99% by weight, from 10% to 99% by weight, from 25% to 99% by weight, from 50% to 99% by weight, or from 75% to 99% by weight. In some embodiments, the percent of lipid in particles can range from 0% to 75% by weight, from 0% to 50% by weight, from 0% to 25% by weight, or from 0% to 10% by weight. In some embodiments, the percent of lipid in particles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

In some embodiments, lipids are oils. In general, any oil known in the art can be included in particles. In some embodiments, an oil may comprise one or more fatty acid groups or salts thereof. In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid group may be a C₁₅-C₂₅ fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

In some embodiments, the oil is a liquid triglyceride.

Suitable oils for use with the present invention include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils, and combinations thereof. Suitable oils for use with the present invention include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In some embodiments, a lipid is a hormone (e.g. estrogen, testosterone), steroid (e.g., cholesterol, bile acid), vitamin (e.g. vitamin E), phospholipid (e.g. phosphatidyl choline), sphingolipid (e.g. ceramides), or lipoprotein (e.g. apolipoprotein).

Carbohydrates

In some embodiments, particles may optionally comprise one or more carbohydrates. The percent of carbohydrate in particles can range from 0% to 99% by weight, from 10% to 99% by weight, from 25% to 99% by weight, from 50% to 99% by weight, or from 75% to 99% by weight. In some embodiments, the percent of carbohydrate in particles can range from 0% to 75% by weight, from 0% to 50% by weight, from 0% to 25% by weight, or from 0% to 10% by weight. In some embodiments, the percent of carbohydrate in particles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

Carbohydrates may be natural or synthetic. A carbohydrate may be a derivatized natural carbohydrate. In certain embodiments, a carbohydrate is a monosaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a disaccharide, including but not limited to lactose, sucrose, maltose, trehalose, and cellobiose. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In certain embodiments, the carbohydrate is a sugar alcohol, including but not limited to mannitol, sorbitol, xylitol, erythritol, maltitol, and lactitol.

### Therapeutic Applications

The compositions and methods described herein can be used for the treatment and/or diagnosis of any disease, disorder, and/or condition which is associated with a tissue specific and/or cell type specific marker (e.g. cancer). Subjects include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Methods of Treatment

In some embodiments, complexes or targeted particles in accordance with the present invention may be used to treat, alleviate, ameliorate, relieve, delay onset of, inhibit progression of, reduce severity of, and/or reduce incidence of one or more symptoms or features of a disease, disorder, and/or condition. In some embodiments, inventive complexes or targeted particles may be used to treat cancer.

Cancer can be associated with a variety of physical symptoms. Symptoms of cancer generally depend on the type, location, and/or stage of the tumor. For example, lung cancer can cause coughing, shortness of breath, and chest pain, while colon cancer often causes diarrhea, constipation, and blood in the stool. However, to give but a few examples, the following symptoms are often generally associated with many cancers: fever, chills, night sweats, cough, dyspnea, weight loss, loss of appetite, anorexia, nausea, vomiting, diarrhea, anemia, jaundice, hepatomegaly, hemoptysis, fatigue, malaise, cognitive dysfunction, depression, hormonal disturbances, neutropenia, pain, non-healing sores, enlarged lymph nodes, peripheral neuropathy, and sexual dysfunction.

In one aspect of the invention, a method for the treatment of cancer (e.g. prostate cancer) is provided. In some embodiments, the treatment of cancer comprises administering a therapeutically effective amount of inventive complexes or targeted particles to a subject in need thereof, in such amounts and for such time as is necessary to achieve the desired result. In certain embodiments of the present invention a “therapeutically effective amount” of an inventive complexes or targeted particle is that amount effective for treating, alleviating, ameliorating, relieving, delaying onset of, inhibiting progression of, reducing severity of, and/or reducing incidence of one or more symptoms or features of cancer.

In one aspect of the invention, a method for administering inventive compositions to a subject suffering from cancer (e.g. prostate cancer) is provided. In some embodiments, such methods comprise administering a therapeutically effective amount of inventive complexes or targeted particles to a subject in such amounts and for such time as is necessary to achieve the desired result (i.e. treatment of cancer). In certain embodiments of the present invention a “therapeutically effective amount” of an inventive complex or targeted particle is that amount effective for treating, alleviating, ameliorating, relieving, delaying onset of, inhibiting progression of, reducing severity of, and/or reducing incidence of one or more symptoms or features of cancer.

Inventive therapeutic protocols involve administering a therapeutically effective amount of an inventive complex or targeted particle to a healthy individual (i.e. a subject who does not display any symptoms of cancer and/or who has not been diagnosed with cancer). For example, healthy individuals may be “immunized” with an inventive complex or targeted particle prior to development of cancer and/or onset of symptoms of cancer; at risk individuals (e.g., patients who have a family history of cancer; patients carrying one or more genetic mutations associated with development of cancer; patients having a genetic polymorphism associated with development of cancer; patients infected by a virus associated with development of cancer; patients with habits and/or lifestyles associated with development of cancer; etc.) can be treated substantially contemporaneously with (e.g., within 48 hours, within 24 hours, or within 12 hours of) the onset of symptoms of cancer. Of course individuals known to have cancer may receive inventive treatment at any time.

The present invention provides methods for treating cancer generally comprising targeted delivery of inventive complexes or targeted particles. Such targeted delivery can be useful for delivery of one or more therapeutic agents that are capable of intercalating between the base pairs of a nucleic acid targeting moiety. Alternatively or additionally, such targeted delivery can be useful for co-delivery of multiple therapeutic agents. For example, targeted particles may comprise at least a second therapeutic agent (e.g. one that is useful for treatment and/or diagnosis of cancer) that is encapsulated within the polymeric matrix of a particle. An example of such a targeted particle that is useful for co-delivery of therapeutic agents (e.g. one agent that is intercalated between the base pairs of the nucleic acid targeting moiety, and one agent that is encapsulated within the polymeric matrix of the particle) is described in Example 3.

Intercalating agents may intercalate between the base pairs of any nucleic acid, such as aptamers, spiegelmers, short interfering RNAs, short hairpin RNAs, micro RNAs, RNAi inducing entities, double-stranded RNAs, etc. In some embodiments, intercalating agents may intercalate between the base pairs of any GC-rich nucleic acid. In some embodiments, intercalating agents may intercalate between the base pairs of any nucleic acid with a high affinity (e.g. high binding coefficient) for the intercalating agent. In some embodiments, intercalating agents may intercalate between the base pairs of nucleic acids that behave as targeting moieties. In some embodiments, intercalating agents may intercalate between the base pairs of nucleic acids that do not behave as targeting moieties. Such complexes can be conjugated to any of the particles described herein. The resulting conjugate may also be associated with a targeting moiety (i.e., not the nucleic acid in which the agent is intercalated), resulting in a targeted particle which is capable of targeted delivery of the agent. Acceptable targeting moieties to be used in such embodiments include, but are not limited to, nucleic acid targeting moieties (e.g. aptamers or spiegelmers), protein targeting moieties (e.g. antibodies), small molecule targeting moieties, carbohydrate targeting moieties, etc. and are described in further detail in co-pending PCT Application U.S. Ser. No. 07/07927, entitled “System for Targeted Delivery of Therapeutic Agents,” filed Mar. 30, 2007.

Encapsulated Agents to be Delivered

According to the present invention, inventive targeted particles may be used for delivery of any agent, including, for example, therapeutic, diagnostic, and/or prophylactic agents. In some embodiments, the therapeutic or diagnostic agent to be delivered is the agent that is intercalated between the base pairs of the nucleic acid targeting moiety. In some embodiments, the agent to be delivered is not the agent that is intercalated between the base pairs of the nucleic acid targeting moiety.

Exemplary agents to be delivered in accordance with the present invention include, but are not limited to, small molecules, organometallic compounds, nucleic acids, proteins (including multimeric proteins, protein complexes, etc.), peptides, lipids, carbohydrates, hormones, metals, radioactive elements and compounds, drugs, vaccines, immunological agents, etc., and/or combinations thereof.

In some embodiments, inventive targeted particles comprise less than 50% by weight, less than 40% by weight, less than 30% by weight, less than 20% by weight, less than 15% by weight, less than 10% by weight, less than 5% by weight, less than 1% by weight, or less than 0.5% by weight of the therapeutic or diagnostic agent to be delivered.

In some embodiments, the agent to be delivered may be a mixture of pharmaceutically active agents. For example, a local anesthetic may be delivered in combination with an anti-inflammatory agent such as a steroid. To give but another example, an antibiotic may be combined with an inhibitor of the enzyme commonly produced by bacteria to inactivate the antibiotic (e.g., penicillin and clavulanic acid).

In some embodiments, the agent to be delivered may be a mixture of anti-cancer agents. In some embodiments, inventive targeted particles are administered in combination with one or more of the anti-cancer agents described herein. Combination therapy is described in further detail below, in the section entitled, “Administration.” To give but one example, in some embodiments, inventive targeted particles comprising a therapeutic or diagnostic agent capable of intercalation between the base pairs of the nucleic acid targeting moiety may be administered in combination with an alkylating agent. To provide another example, inventive compositions comprising an anti-cancer agent to be delivered are administered in combination with hormonal therapy. The growth of some types of tumors can be inhibited by providing or blocking certain hormones. For example, steroids (e.g. dexamethasone) can inhibit tumor growth or associated edema and may cause regression of lymph node malignancies. In some cases, prostate cancer is often sensitive to finasteride, an agent that blocks the peripheral conversion of testosterone to dihydrotestosterone. Breast cancer cells often highly express the estrogen and/or progesterone receptor Inhibiting the production (e.g. with aromatase inhibitors) or function (e.g. with tamoxifen) of these hormones can often be used in breast cancer treatments. In some embodiments, gonadotropin-releasing hormone agonists (GnRH), such as goserelin possess a paradoxic negative feedback effect followed by inhibition of the release of follicle stimulating hormone (FSH) and leuteinizing hormone (LH), when given continuously.

A. Small Molecule Agents

In some embodiments, the agent to be delivered is a small molecule and/or organic compound with pharmaceutical activity. In some embodiments, the agent is a clinically-used drug. In some embodiments, the drug is an anti-cancer agent, antibiotic, anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-Parkinson agent, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g. cell growth inhibitors and anti-adhesion molecules), inhibitors of DNA, RNA, or protein synthesis, etc.

In certain embodiments, the therapeutic agent to be delivered is an anti-cancer agent (i.e. cytotoxic agents). Most anti-cancer agents can be divided in to the following categories: alkylating agents, antimetabolites, natural products, and hormones and antagonists.

Anti-cancer agents typically affect cell division and/or DNA synthesis. However, some chemotherapeutic agents do not directly interfere with DNA. To give but one example, tyrosine kinase inhibitors (imatinib mesylate/Gleevec®) directly target a molecular abnormality in certain types of cancer (chronic myelogenous leukemia, gastrointestinal stromal tumors, etc.).

Alkylating agents are so named because of their ability to add alkyl groups to many electronegative groups under conditions present in cells. Alkylating agents typically function by chemically modifying cellular DNA. Exemplary alkylating agents include nitrogen mustards (e.g. mechlorethamine, cyclophosphamide, ifosfamide, melphalan (1-sarcolysin), chlorambucil), ethylenimines and methylmelamines (e.g. altretamine (hexamethylmelamine; HMM), thiotepa (triethylene thiophosphoramide), triethylenemelamine (TEM)), alkyl sulfonates (e.g. busulfan), nitrosureas (e.g. carmustine (BCNU), lomustine (CCMU), semustine (methyl-CCNU), streptozocin (streptozotocin)), and triazenes (e.g. dacarbazine (DTIC; dimethyltriazenoimidazolecarboxamide)).

Antimetabolites act by mimicking small molecule metabolites (e.g. folic acid, pyrimidines, and purines) in order to be incorporated into newly synthesized cellular DNA. Such agents also affect RNA synthesis. An exemplary folic acid analog is methotrexate (amethopterin). Exemplary pyrimidine analogs include fluorouracil (5-fluorouracil; 5-FU), floxuridine (fluorodeoxyuridine; FUdR), and cytarabine (cytosine arabinoside). Exemplary purine analogs include mercaptopurine (6-mercaptopurine; 6-MP), azathioprine, thioguanine (6-thioguanine; TG), fludarabine phosphate, pentostatin (2′-deoxycoformycin), cladribine (2-chlorodeoxyadenosine; 2-CdA), and erythrohydroxynonyladenine (EHNA).

Natural small molecule products which can be used as anti-cancer agents include plant alkaloids and antibiotics. Plant alkaloids and terpenoids (e.g. vinca alkaloids, podophyllotoxin, taxanes, etc.) typically block cell division by preventing microtubule function. Vinca alkaloids (e.g. vincristine, vinblastine (VLB), vinorelbine, vindesine, etc.) bind to tubulin and inhibit assembly of tubulin into microtubules. Vinca alkaloids are derived from the Madagascar periwinkle, Catharanthus roseus (formerly known as Vinca rosea). Podophyllotoxin is a plant-derived compound used to produce two other cytostatic therapeutic agents, etoposide and teniposide, which prevent cells from entering the G1 and S phases of the cell cycle. Podophyllotoxin is primarily obtained from the American Mayapple (Podophyllum peltatum) and a Himalayan Mayapple (Podophyllum hexandrum). Taxanes (e.g. paclitaxel, docetaxel, etc.) are derived from the Yew Tree. Taxanes enhance stability of microtubules, preventing the separation of chromosomes during anaphase.

Antibiotics which can be used as anti-cancer agents include dactinomycin (actinomycin D), daunorubicin (daunomycin; rubidomycin), doxorubicin, idarubicin, bleomycin, plicamycin (mithramycin), and mitomycin (mytomycin C).

Other small molecules which can be used as anti-cancer agents include platinum coordination complexes (e.g. cisplatin (cis-DDP), carboplatin), anthracenedione (e.g. mitoxantrone), substituted urea (e.g. hydroxyurea), methylhydrazine derivatives (e.g. procarbazine (N-methylhydrazine, MIH), and adrenocortical suppressants (e.g. mitotane (o,p′-DDD), aminoglutethimide).

Hormones which can be used as anti-cancer agents include adrenocorticosteroids (e.g. prednisone), aminoglutethimide, progestins (e.g. hydroxyprogesterone caproate, medroxyprogesterone acetate, megestrol acetate), estrogens (e.g. diethylstilbestrol, ethinyl estradiol), antiestrogen (e.g. tamoxifen), androgens (e.g. testosterone propionate, fluoxymesterone), antiandrogens (e.g. flutamide), and gonadotropin-releasing hormone analog (e.g. leuprolide).

Topoisomerase inhibitors act by inhibiting the function of topoisomerases, which are enzymes that maintain the topology of DNA Inhibition of type I or type II topoisomerases interferes with both transcription and replication of DNA by upsetting proper DNA supercoiling. Some exemplary type I topoisomerase inhibitors include camptothecins (e.g. irinotecan, topotecan, etc.). Some exemplary type II topoisomerase inhibitors include amsacrine, etoposide, etoposide phosphate, teniposide, etc., which are semisynthetic derivatives of epipodophyllotoxins, discussed herein.

In certain embodiments, a small molecule agent can be any drug. In some embodiments, the drug is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention.

A more complete listing of classes and specific drugs suitable for use in the present invention may be found in Pharmaceutical Drugs: Syntheses, Patents, Applications by Axel Kleemann and Jurgen Engel, Thieme Medical Publishing, 1999 and the Merck Index: An Encyclopedia of Chemicals, Drugs and Biologicals, Ed. by Budavari et al., CRC Press, 1996, both of which are incorporated herein by reference.

B. Nucleic Acid Agents

In certain embodiments of the invention, an inventive targeted particle is used to deliver one or more nucleic acids (e.g. functional RNAs, functional DNAs, etc.) to a specific location such as an organ, tissue, cell, extracellular matrix component, and/or intracellular compartment.

Functional RNA

In general, a “functional RNA” is an RNA that does not code for a protein but instead belongs to a class of RNA molecules whose members characteristically possess one or more different functions or activities within a cell. It will be appreciated that the relative activities of functional RNA molecules having different sequences may differ and may depend at least in part on the particular cell type in which the RNA is present. Thus the term “functional RNA” is used herein to refer to a class of RNA molecule and is not intended to imply that all members of the class will in fact display the activity characteristic of that class under any particular set of conditions. In some embodiments, functional RNAs include RNAi-inducing entities (e.g. short interfering RNAs (siRNAs), short hairpin RNAs (shRNAs), and microRNAs), ribozymes, tRNAs, rRNAs, RNAs useful for triple helix formation, etc.

RNAi is an evolutionarily conserved process in which presence of an at least partly double-stranded RNA molecule in a eukaryotic cell leads to sequence-specific inhibition of gene expression. RNAi was originally described as a phenomenon in which the introduction of long dsRNA (typically hundreds of nucleotides) into a cell results in degradation of mRNA containing a region complementary to one strand of the dsRNA (U.S. Pat. No. 6,506,559; and Fire et al., 1998, Nature, 391:806). Subsequent studies in Drosophila showed that long dsRNAs are processed by an intracellular RNase III-like enzyme called Dicer into smaller dsRNAs primarily comprised of two approximately 21 nucleotide (nt) strands that form a 19 base pair duplex with 2 nt 3′ overhangs at each end and 5′-phosphate and 3′-hydroxyl groups (see, e.g., PCT Publication WO 01/75164; U.S. Patent Application Publications 2002/0086356 and 2003/0108923; Zamore et al., 2000, Cell, 101:25; and Elbashir et al., 2001, Genes Dev., 15:188).

Short dsRNAs having structures such as this, referred to as siRNAs, silence expression of genes that include a region that is substantially complementary to one of the two strands. This strand is referred to as the “antisense” or “guide” strand, with the other strand often being referred to as the “sense” strand. The siRNA is incorporated into a ribonucleoprotein complex termed the RNA-induced silencing complex (RISC) that contains member(s) of the Argonaute protein family. Following association of the siRNA with RISC, a helicase activity unwinds the duplex, allowing an alternative duplex to form the guide strand and a target mRNA containing a portion substantially complementary to the guide strand. An endonuclease activity associated with the Argonaute protein(s) present in RISC is responsible for “slicing” the target mRNA, which is then further degraded by cellular machinery.

Considerable progress towards the practical application of RNAi was achieved with the discovery that exogenous introduction of siRNAs into mammalian cells can effectively reduce the expression of target genes in a sequence-specific manner via the mechanism described above. A typical siRNA structure includes a 19 nucleotide double-stranded portion, comprising a guide strand and an antisense strand. Each strand has a 2 nt 3′ overhang. Typically the guide strand of the siRNA is perfectly complementary to its target gene and mRNA transcript over at least 17-19 contiguous nucleotides, and typically the two strands of the siRNA are perfectly complementary to each other over the duplex portion. However, as will be appreciated by one of ordinary skill in the art, perfect complementarity is not required. Instead, one or more mismatches in the duplex formed by the guide strand and the target mRNA is often tolerated, particularly at certain positions, without reducing the silencing activity below useful levels. For example, there may be 1, 2, 3, or even more mismatches between the target mRNA and the guide strand (disregarding the overhangs). Thus, as used herein, two nucleic acid portions such as a guide strand (disregarding overhangs) and a portion of a target mRNA that are “substantially complementary” may be perfectly complementary (i.e., they hybridize to one another to form a duplex in which each nucleotide is a member of a complementary base pair) or they may have a lesser degree of complementarity sufficient for hybridization to occur. One of ordinary skill in the art will appreciate that the two strands of the siRNA duplex need not be perfectly complementary. Typically at least 80%, preferably at least 90%, or more of the nucleotides in the guide strand of an effective siRNA are complementary to the target mRNA over at least about 19 contiguous nucleotides. The effect of mismatches on silencing efficacy and the locations at which mismatches may most readily be tolerated are areas of active study (see, e.g., Reynolds et al., 2004, Nat. Biotechnol., 22:326).

It will be appreciated that molecules having the appropriate structure and degree of complementarity to a target gene will exhibit a range of different silencing efficiencies. A variety of additional design criteria have been developed to assist in the selection of effective siRNA sequences. Numerous software programs that can be used to choose siRNA sequences that are predicted to be particularly effective to silence a target gene of choice are available (see, e.g., Yuan et al., 2004, Nucl. Acids. Res., 32:W130; and Santoyo et al., 2005, Bioinformatics, 21:1376).

As will be appreciated by one of ordinary skill in the art, RNAi may be effectively mediated by RNA molecules having a variety of structures that differ in one or more respects from that described above. For example, the length of the duplex can be varied (e.g., from about 17-29 nucleotides); the overhangs need not be present and, if present, their length and the identity of the nucleotides in the overhangs can vary (though most commonly symmetric dTdT overhangs are employed in synthetic siRNAs).

Additional structures, referred to as short hairpin RNAs (shRNAs), are capable of mediating RNA interference. An shRNA is a single RNA strand that contains two complementary regions that hybridize to one another to form a double-stranded “stem,” with the two complementary regions being connected by a single-stranded loop. shRNAs are processed intracellularly by Dicer to form an siRNA structure containing a guide strand and an antisense strand. While shRNAs can be delivered exogenously to cells, more typically intracellular synthesis of shRNA is achieved by introducing a plasmid or vector containing a promoter operably linked to a template for transcription of the shRNA into the cell, e.g., to create a stable cell line or transgenic organism.

While sequence-specific cleavage of target mRNA is currently the most widely used means of achieving gene silencing by exogenous delivery of short RNAi entities to cells, additional mechanisms of sequence-specific silencing mediated by short RNA entities are known. For example, post-transcriptional gene silencing mediated by small RNA entities can occur by mechanisms involving translational repression. Certain endogenously expressed RNA molecules form hairpin structures containing an imperfect duplex portion in which the duplex is interrupted by one or more mismatches and/or bulges. These hairpin structures are processed intracellularly to yield single-stranded RNA species referred to as known as microRNAs (miRNAs), which mediate translational repression of a target transcript to which they hybridize with less than perfect complementarity. siRNA-like molecules designed to mimic the structure of miRNA precursors have been shown to result in translational repression of target genes when administered to mammalian cells.

Thus the exact mechanism by which a short RNAi entity inhibits gene expression appears to depend, at least in part, on the structure of the duplex portion of the RNAi entity and/or the structure of the hybrid formed by one strand of the RNAi entity and a target transcript. RNAi mechanisms and the structure of various RNA molecules known to mediate RNAi, e.g., siRNA, shRNA, miRNA and their precursors, have been extensively reviewed (see, e.g., Dykxhhorn et al., 2003, Nat. Rev. Mol. Cell. Biol., 4:457; Hannon et al., 2004, Nature, 431:3761; and Meister et al., 2004, Nature, 431:343). It is to be expected that future developments will reveal additional mechanisms by which RNAi may be achieved and will reveal additional effective short RNAi entities. Any currently known or subsequently discovered short RNAi entities are within the scope of the present invention.

A short RNAi entity that is delivered according to the methods of the invention and/or is present in a composition of the invention may be designed to silence any eukaryotic gene. The gene can be a mammalian gene, e.g., a human gene. The gene can be a wild type gene, a mutant gene, an allele of a polymorphic gene, etc. The gene can be disease-associated, e.g., a gene whose over-expression, under-expression, or mutation is associated with or contributes to development or progression of a disease. For example, the gene can be oncogene. The gene can encode a receptor or putative receptor for an infectious agent such as a virus (see, e.g., Dykxhhorn et al., 2003, Nat. Rev. Mol. Cell. Biol., 4:457 for specific examples).

In some embodiments, tRNAs are functional RNA molecules whose delivery to eukaryotic cells can be monitored using the compositions and methods of the invention. The structure and role of tRNAs in protein synthesis is well known (Soll and Rajbhandary, (eds.) tRNA: Structure, Biosynthesis, and Function, ASM Press, 1995). The cloverleaf shape of tRNAs includes several double-stranded “stems” that arise as a result of formation of intramolecular base pairs between complementary regions of the single tRNA strand. There is considerable interest in the synthesis of polypeptides that incorporate unnatural amino acids such as amino acid analogs or labeled amino acids at particular positions within the polypeptide chain (see, e.g., Köhrer and RajBhandary, “Proteins carrying one or more unnatural amino acids,” Chapter 33, In Ibba et al., (eds.), Aminoacyl-tRNA Synthetases, Landes Bioscience, 2004). One approach to synthesizing such polypeptides is to deliver a suppressor tRNA that is aminoacylated with an unnatural amino acid to a cell that expresses an mRNA that encodes the desired polypeptide but includes a nonsense codon at one or more positions. The nonsense codon is recognized by the suppressor tRNA, resulting in incorporation of the unnatural amino acid into a polypeptide encoded by the mRNA (Kohrer et al., 2001, Proc. Natl. Acad. Sci., USA, 98:14310; and Kohrer et al., 2004, Nucleic Acids Res., 32:6200). However, as in the case of siRNA delivery, existing methods of delivering tRNAs to cells result in variable levels of delivery, complicating efforts to analyze such proteins and their effects on cells.

The invention contemplates the delivery of tRNAs, e.g., suppressor tRNAs, and optically or magnetically detectable particles to eukaryotic cells in order to achieve the synthesis of proteins that incorporate an unnatural amino acid with which the tRNA is aminoacylated. The analysis of proteins that incorporate one or more unnatural amino acids has a wide variety of applications. For example, incorporation of amino acids modified with detectable (e.g., fluorescent) moieties can allow the study of protein trafficking, secretion, etc., with minimal disturbance to the native protein structure. Alternatively or additionally, incorporation of reactive moieties (e.g., photoactivatable and/or cross-linkable groups) can be used to identify protein interaction partners and/or to define three-dimensional structural motifs. Incorporation of phosphorylated amino acids such as phosphotyrosine, phosphothreonine, or phosphoserine, or analogs thereof, into proteins can be used to study cell signaling pathways and requirements.

In one embodiment of the invention, the functional RNA is a ribozyme. A ribozyme is designed to catalytically cleave target mRNA transcripts may be used to prevent translation of a target mRNA and/or expression of a target (see, e.g., PCT publication WO 90/11364; and Sarver et al., 1990, Science 247:1222).

In some embodiments, endogenous target gene expression may be reduced by targeting deoxyribonucleotide sequences complementary to the regulatory region of the target gene (i.e., the target gene's promoter and/or enhancers) to form triple helical structures that prevent transcription of the target gene in target muscle cells in the body (see generally, Helene, 1991, Anticancer Drug Des. 6:569; Helene et al., 1992, Ann, N.Y. Acad. Sci. 660:27; and Maher, 1992, Bioassays 14:807).

RNAs such as RNAi-inducing entities, tRNAs, ribozymes, etc., for delivery to eukaryotic cells may be prepared according to any available technique including, but not limited to chemical synthesis, enzymatic synthesis, enzymatic or chemical cleavage of a longer precursor, etc. Methods of synthesizing RNA molecules are known in the art (see, e.g., Gait, M. J. (ed.) Oligonucleotide synthesis: a practical approach, Oxford (Oxfordshire), Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005). Short RNAi entities such as siRNAs are commercially available from a number of different suppliers. Pre-tested siRNAs targeted to a wide variety of different genes are available, e.g., from Ambion (Austin, Tex.), Dharmacon (Lafayette, Colo.), Sigma-Aldrich (St. Louis, Mo.).

When siRNAs are synthesized in vitro the two strands are typically allowed to hybridize before contacting them with cells. It will be appreciated that the resulting siRNA composition need not consist entirely of double-stranded (hybridized) molecules. For example, an RNAi entity commonly includes a small proportion of single-stranded RNA. Generally, at least approximately 50%, at least approximately 90%, at least approximately 95%, or even at least approximately 99%-100% of the RNAs in an siRNA composition are double-stranded when contacted with cells. However, a composition containing a lower proportion of dsRNA may be used, provided that it contains sufficient dsRNA to be effective.

Vectors

In some embodiments, a nucleic acid to be delivered is a vector. As used herein, the term “vector” refers to a nucleic acid molecule (typically, but not necessarily, a DNA molecule) which can transport another nucleic acid to which it has been linked. A vector can achieve extra-chromosomal replication and/or expression of nucleic acids to which they are linked in a host cell (e.g. a cell targeted by targeted particles of the present invention). In some embodiments, a vector can achieve integration into the genome of the host cell.

In some embodiments, vectors are used to direct protein and/or RNA expression. In some embodiments, the protein and/or RNA to be expressed is not normally expressed by the cell. In some embodiments, the protein and/or RNA to be expressed is normally expressed by the cell, but at lower levels than it is expressed when the vector has not been delivered to the cell.

In some embodiments, a vector directs expression of any of the proteins described herein. In some embodiments, a vector directs expression of a protein with anti-cancer activity. In some embodiments, a vector directs expression of any of the functional RNAs described herein, such as RNAi-inducing entities, ribozymes, etc. In some embodiments, a vector directs expression of a functional RNA with anti-cancer activity.

C. Protein Agents

In some embodiments, the agent to be delivered may be a protein or peptide. In certain embodiments, peptides range from about 5 to about 5000, 5 to about 1000, about 5 to about 750, about 5 to about 500, about 5 to about 250, about 5 to about 100, about 5 to about 75, about 5 to about 50, about 5 to about 40, about 5 to about 30, about 5 to about 25, about 5 to about 20, about 5 to about 15, or about 5 to about 10 amino acids in size. Peptides from panels of peptides comprising random sequences and/or sequences which have been varied consistently to provide a maximally diverse panel of peptides may be used.

The terms “protein,” “polypeptide,” and “peptide” are used interchangeably herein, typically referring to a polypeptide having a length of less than about 500 to about 1000 amino acids. Polypeptides may contain L-amino acids, D-amino acids, or both and may contain any of a variety of amino acid modifications or analogs known in the art. Useful modifications include, e.g., terminal acetylation, amidation, etc. In some embodiments, polypeptides may comprise natural amino acids, unnatural amino acids, synthetic amino acids, and combinations thereof, as described herein.

In some embodiments, the agent to be delivered may be a peptide, hormone, erythropoietin, insulin, cytokine, antigen for vaccination, etc. In some embodiments, the agent to be delivered may be an antibody and/or characteristic portion thereof. In some embodiments, antibodies may include, but are not limited to, polyclonal, monoclonal, chimeric (i.e. “humanized”), single chain (recombinant) antibodies. In some embodiments, antibodies may have reduced effector functions and/or bispecific molecules. In some embodiments, antibodies may include Fab fragments and/or fragments produced by a Fab expression library, as described in further detail above.

In some embodiments, the agent to be delivered may be an anti-cancer agent. Exemplary protein anti-cancer agents are enzymes (e.g. L-asparaginase) and biological response modifiers, such as interferons (e.g. interferon-α), interleukins (e.g. interleukin 2; IL-2), granulocyte colony-stimulating factor (G-CSF), and granulocyte/macrophage colony-stimulating factor (GM-CSF). In some embodiments, a protein anti-cancer agent is an antibody or characteristic portion thereof which is cytotoxic to tumor cells.

D. Carbohydrate Agents

In some embodiments, the agent to be delivered is a carbohydrate, such as a carbohydrate that is associated with a protein (e.g. glycoprotein, proteogycan, etc.). A carbohydrate may be natural or synthetic. A carbohydrate may also be a derivatized natural carbohydrate. In certain embodiments, a carbohydrate may be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, malitol, and lactitol.

E. Lipid Agents

In some embodiments, the agent to be delivered is a lipid, such as a lipid that is associated with a protein (e.g. lipoprotein). Exemplary lipids that may be used in accordance with the present invention include, but are not limited to, oils, fatty acids, saturated fatty acid, unsaturated fatty acids, essential fatty acids, cis fatty acids, trans fatty acids, glycerides, monoglycerides, diglycerides, triglycerides, hormones, steroids (e.g., cholesterol, bile acids), vitamins (e.g. vitamin E), phospholipids, sphingolipids, and lipoproteins.

In some embodiments, the lipid may comprise one or more fatty acid groups or salts thereof. In some embodiments, the fatty acid group may comprise digestible, long chain (e.g., C₈-C₅₀), substituted or unsubstituted hydrocarbons. In some embodiments, the fatty acid group may be a C₁₀-C₂₀ fatty acid or salt thereof. In some embodiments, the fatty acid group may be a C₁₅-C₂₀ fatty acid or salt thereof. In some embodiments, the fatty acid group may be a C₁₅-C₂₅ fatty acid or salt thereof. In some embodiments, the fatty acid group may be unsaturated. In some embodiments, the fatty acid group may be monounsaturated. In some embodiments, the fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, the fatty acid group may be one or more of butyric, caproic, caprylic, capric, lauric, myristic, palmitic, stearic, arachidic, behenic, or lignoceric acid. In some embodiments, the fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

F. Diagnostic Agents

In some embodiments, the agent to be delivered is a diagnostic agent. In some embodiments, diagnostic agents include gases; commercially available imaging agents used in positron emissions tomography (PET), computer assisted tomography (CAT), single photon emission computerized tomography, x-ray, fluoroscopy, and magnetic resonance imaging (MRI); anti-emetics; and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

In some embodiments, inventive targeted particles may comprise a diagnostic agent used in magnetic resonance imaging (MRI), such as iron oxide particles or gadolinium complexes. Gadolinium complexes that have been approved for clinical use include gadolinium chelates with DTPA, DTPA-BMA, DOTA and HP-DO3A (reviewed in Aime et al., 1998, Chemical Society Reviews, 27:19).

In some embodiments, inventive targeted particles may comprise radionuclides as therapeutic and/or diagnostic agents. Among the radionuclides used, gamma-emitters, positron-emitters, and X-ray emitters are suitable for diagnostic and/or therapy, while beta emitters and alpha-emitters may also be used for therapy. Suitable radionuclides for forming the targeted particle of the invention include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ⁹⁹mTc, ¹⁴C, ¹³N, ¹⁵O, ³²P, ³³P, and ¹⁸F.

In some embodiments, a diagnostic agent may be a fluorescent, luminescent, or magnetic moiety. In some embodiments, a detectable moiety such as a fluorescent or luminescent dye, etc., is entrapped, embedded, or encapsulated by a particle core and/or coating layer.

Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., U.S. Patent Application Publication 2004/0067503; Valeur, B., “Molecular Fluorescence: Principles and Applications,” John Wiley and Sons, 2002; Handbook of Fluorescent Probes and Research Products, Molecular Probes, 9^{th} edition, 2002; and The Handbook—A Guide to Fluorescent Probes and Labeling Technologies, Invitrogen, 10^{th} edition, available at the Invitrogen web site).

G. Prophylactic Agents

In some embodiments, the agent to be delivered is a prophylactic agent. In some embodiments, prophylactic agents include vaccines. Vaccines may comprise isolated proteins or peptides, inactivated organisms and viruses, dead organisms and virus, genetically altered organisms or viruses, and cell extracts. Prophylactic agents may be combined with interleukins, interferon, cytokines, and adjuvants such as cholera toxin, alum, Freund's adjuvant, etc. Prophylactic agents may include antigens of such bacterial organisms as Streptococcus pnuemoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

H. Nutraceutical Agents

In some embodiments, the therapeutic agent to be delivered is a nutraceutical agent. In some embodiments, the nutraceutical agent provides basic nutritional value, provides health or medical benefits, and/or is a dietary supplement. In some embodiments, the nutraceutical agent is a vitamin (e.g. vitamins A, B, C, D, E, K, etc.), mineral (e.g. iron, magnesium, potassium, calcium, etc.), or essential amino acid (e.g. lysine, glutamine, leucine, etc.).

In some embodiments, nutraceutical agents may include plant or animal extracts, such as fatty acids and/or omega-3 fatty acids (e.g. DHA or ARA), fruit and vegetable extracts, lutein, phosphatidylserine, lipoid acid, melatonin, glucosamine, chondroitin, aloe vera, guggul, green tea, lycopene, whole foods, food additives, herbs, phytonutrients, antioxidants, flavonoid constituents of fruits, evening primrose oil, flaxseeds, fish and marine animal oils (e.g. cod liver oil), and probiotics.

Exemplary nutraceutical agents and dietary supplements are disclosed, for example, in Roberts et al., (Nutriceuticals: The Complete Encyclopedia of Supplements, Herbs, Vitamins, and Healing Foods, American Nutriceutical Association, 2001). Nutraceutical agents and dietary supplements are also disclosed in Physicians' Desk Reference for Nutritional Supplements, 1st Ed. (2001) and The Physicians' Desk Reference for Herbal Medicines, 1st Ed. (2001).

Those skilled in the art will recognize that this is an exemplary, not comprehensive, list of therapeutic or diagnostic agents that can be delivered using the targeted particles of the present invention. Any therapeutic or diagnostic agent may be associated with particles for targeted delivery in accordance with the present invention.

Methods of Diagnosis

In some embodiments, targeted particles of the present invention may be used to diagnose a disease, disorder, and/or condition (e.g., autoimmune disorders; inflammatory disorders; infectious diseases; neurological disorders; cardiovascular disorders; proliferative disorders; respiratory disorders; digestive disorders; musculoskeletal disorders; endocrine, metabolic, and nutritional disorders; urological disorders; psychological disorders; skin disorders; blood and lymphatic disorders; etc.). In some embodiments, inventive targeted particles may be used to diagnose cancer. In some embodiments, such methods of diagnosis may involve the use of inventive targeted particles to physically detect and/or locate a tumor within the body of a subject.

In one aspect of the invention, a method for the diagnosis of cancer (e.g. prostate cancer) is provided. In some embodiments, the diagnosis of cancer comprises administering a therapeutically effective amount of inventive targeted particles to a subject, in such amounts and for such time as is necessary to achieve the desired result. In certain embodiments of the present invention a “therapeutically effective amount” of an inventive targeted particle is that amount effective for diagnosing cancer.

In some embodiments, inventive targeted particles comprise particles which have intrinsically detectable properties (described in further detail below). In some embodiments, inventive targeted particles comprise particles which do not have intrinsically detectable properties but are associated with a substance which is detectable. Such targeted particles are capable of simultaneously diagnosing and treating cancer. In particular, such targeted particles are capable of treating cancer by delivery of the agent that is intercalated between the base pairs of the nucleic acid targeting moiety, and such targeted particles are capable of diagnosing cancer by delivery of a detectable particle to the site of a tumor.

Targeted Particles Comprising a Detectable Agent

In certain embodiments of the invention, the particle comprises a bulk material that is not intrinsically detectable. The particle comprises one or more fluorescent, luminescent, or magnetic moieties. For example, the particle may comprise fluorescent or luminescent substances or smaller particles of a magnetic material. In some embodiments, an optically detectable moiety such as a fluorescent or luminescent dye, etc., is entrapped, embedded, or encapsulated by a particle core and/or coating layer. Fluorescent and luminescent moieties include a variety of different organic or inorganic small molecules, as described in further detail above.

Fluorescence or luminescence can be detected using any approach known in the art including, but not limited to, spectrometry, fluorescence microscopy, flow cytometry, etc. Spectrofluorometers and microplate readers are typically used to measure average properties of a sample while fluorescence microscopes resolve fluorescence as a function of spatial coordinates in two or three dimensions for microscopic objects (e.g., less than approximately 0.1 mm diameter). Microscope-based systems are thus suitable for detecting and optionally quantitating particles inside individual cells.

Flow cytometry measures properties such as light scattering and/or fluorescence on individual cells in a flowing stream, allowing subpopulations within a sample to be identified, analyzed, and optionally quantitated (see, e.g., Mattheakis et al., 2004, Analytical Biochemistry, 327:200). Multiparameter flow cytometers are available. In certain embodiments of the invention, laser scanning cytometery is used (Kamentsky, 2001, Methods Cell Biol., 63:51). Laser scanning cytometry can provide equivalent data to a flow cytometer but is typically applied to cells on a solid support such as a slide. It allows light scatter and fluorescence measurements and records the position of each measurement. Cells of interest may be re-located, visualized, stained, analyzed, and/or photographed. Laser scanning cytometers are available, e.g., from CompuCyte (Cambridge, Mass.).

In certain embodiments of the invention, an imaging system comprising an epifluorescence microscope equipped with a laser (e.g., a 488 nm argon laser) for excitation and appropriate emission filter(s) is used. The filters should allow discrimination between different populations of particles used in the particular assay. For example, in one embodiment, the microscope is equipped with fifteen 10 nm bandpass filters spaced to cover portion of the spectrum between 520 and 660 nm, which would allow the detection of a wide variety of different fluorescent particles. Fluorescence spectra can be obtained from populations of particles using a standard UV/visible spectrometer.

Targeted Particles Comprising Particles with Intrinsically Detectable Properties

In some embodiments, particles have detectable optical and/or magnetic properties, though particles that may be detected by other approaches could be used. An optically detectable particle is one that can be detected within a living cell using optical means compatible with cell viability. Optical detection is accomplished by detecting the scattering, emission, and/or absorption of light that falls within the optical region of the spectrum, i.e., that portion of the spectrum extending from approximately 180 nm to several microns. Optionally a sample containing cells is exposed to a source of electromagnetic energy. In some embodiments of the invention, absorption of electromagnetic energy (e.g., light of a given wavelength) by the particle or a component thereof is followed by the emission of light at longer wavelengths, and the emitted light is detected. In some embodiments, scattering of light by the particles is detected. In certain embodiments of the invention, light falling within the visible portion of the electromagnetic spectrum, i.e., the portion of the spectrum that is detectable by the human eye (approximately 400 nm to approximately 700 nm) is detected. In some embodiments of the invention, light that falls within the infrared or ultraviolet region of the spectrum is detected.

An optical property can be a feature of an absorption, emission, or scattering spectrum or a change in a feature of an absorption, emission, or scattering spectrum. An optical property can be a visually detectable feature such as, for example, color, apparent size, or visibility (i.e. simply whether or not the particle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the particles; thus polarization dependence can be measured. Features associated with hyper-Rayleigh scattering can be measured. Fluorescence detection can include detection of fluorescence modes and any of the methods described herein.

Intrinsically fluorescent or luminescent particles, particles that comprise fluorescent or luminescent moieties, plasmon resonant particles, and magnetic particles are among the detectable particles that are used in various embodiments of the invention. Such particles can have a variety of different shapes including spheres, oblate spheroids, cylinders, shells, cubes, pyramids, rods (e.g., cylinders or elongated structures having a square or rectangular cross-section), tetrapods (particles having four leg-like appendages), triangles, prisms, etc. In general, the particles should have dimensions small enough to allow their uptake by eukaryotic cells. Typically the particles have a longest straight dimension (e.g., diameter) of 200 nm or less. In some embodiments, the particles have a diameter of 100 nm or less. Smaller particles, e.g., having diameters of 50 nm or less, e.g., 5-30 nm, are used in some embodiments of the invention. In some embodiments, the term “particle” encompasses atomic clusters, which have a typical diameter of 1 nm or less and generally contain from several (e.g., 3-4) up to several hundred atoms.

In certain embodiments of the invention, the particles can be quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible (see, e.g., Zheng et al., 2004, Phys. Rev. Lett., 93:7, describing gold QDs). By varying their size and composition, the emission wavelength can be tuned (i.e., adjusted in a predictable and controllable manner) from the blue to the near infrared. QDs generally have a broad absorption spectrum and a narrow emission spectrum. Thus different QDs having distinguishable optical properties (e.g., peak emission wavelength) can be excited using a single source. QDs are brighter than most conventional fluorescent dyes by approximately 10-fold (Wu et al., 2003, Nat. Biotechnol., 21:41; and Gao et al., 2004, Nat. Biotechnol., 22:969) and have been significantly easier to detect than GFP among background autofluorescence in vivo (Gao et al., 2004, Nat. Biotechnol., 22:969). Furthermore, QDs are less susceptible to photobleaching, fluorescing more than 20 times longer than conventional fluorescent dyes under continuous mercury lamp exposure (Derfus et al., 2004, Advanced Materials, 16:961).

In certain embodiments of the invention, optically detectable particles are metal particles. Metals of use in the particles include, but are not limited to, gold, silver, iron, cobalt, zinc, cadmium, nickel, gadolinium, chromium, copper, manganese, palladium, tin, and alloys thereof. Oxides of any of these metals can be used.

Noble metals (e.g., gold, silver, copper, platinum, palladium) are preferred for plasmon resonant particles, which are discussed in further detail below. For example, gold, silver, or an alloy comprising gold, silver, and optionally one or more other metals can be used. Core/shell particles (e.g., having a silver core with an outer shell of gold, or vice versa) can be used. Particles containing a metal core and a nonmetallic inorganic or organic outer shell, or vice versa, can be used. In certain embodiments, the nonmetallic core or shell comprises a dielectric material such as silica. Composite particles in which a plurality of metal particles are embedded or trapped in a nonmetal (e.g., a polymer or a silica shell) may be used. Hollow metal particles (e.g., hollow nanoshells) having an interior space or cavity are used in some embodiments. In some embodiments, a nanoshell comprising two or more concentric hollow spheres is used. Such a particle optionally comprises a core, e.g., made of a dielectric material.

In certain embodiments of the invention, at least 1%, or typically at least 5% of the mass or volume of the particle or number of atoms in the particle is contributed by metal atoms. In certain embodiments of the invention, the amount of metal in the particle, or in a core or coating layer comprising a metal, ranges from approximately 5% to 100% by mass, volume, or number of atoms, or can assume any value or range between 5 and 100%.

Certain metal particles, referred to as plasmon resonant particles, exhibit the well known phenomenon of plasmon resonance. When a metal particle (usually made of a noble metal such as gold, silver, copper, platinum, etc.) is subjected to an external electric field, its conduction electrons are displaced from their equilibrium positions with respect to the nuclei, which in turn exert an attractive, restoring force. If the electric field is oscillating (as in the case of electromagnetic radiation such as light), the result is a collective oscillation of the conduction electrons in the particle, known as plasmon resonance (Kelly et al., 2003, J. Phys. Chem. B., 107:668; Schultz et al., 2000, Proc. Natl. Acad. Sci., USA, 97:996; and Schultz, 2003, Curr. Op. Biotechnol., 14:13). The plasmon resonance phenomenon results in extremely efficient wavelength-dependent scattering and absorption of light by the particles over particular bands of frequencies, often in the visible range. Scattering and absorption give rise to a number of distinctive optical properties that can be detected using various approaches including visually (i.e., by the naked eye or using appropriate microscopic techniques) and/or by obtaining a spectrum, e.g., a scattering spectrum, extinction (scattering+absorption) spectrum, or absorption spectrum from the particle(s).

Certain lanthanide ion-doped particles exhibit strong fluorescence and are of use in certain embodiments of the invention. A variety of different dopant molecules can be used. For example, fluorescent europium-doped yttrium vanadate (YVO₄) particles have been produced (Beaureparie et al., 2004, Nano Letters, 4:2079). These particles may be synthesized in water and are readily functionalized with biomolecules.

Magnetic particles are of use in the invention. “Magnetic particles” refers to magnetically responsive particles that contain one or more metals or oxides or hydroxides thereof. Such particles typically react to magnetic force resulting from a magnetic field. The field can attract or repel the particle towards or away from the source of the magnetic field, respectively, optionally causing acceleration or movement in a desired direction in space. A magnetically detectable particle is a magnetic particle that can be detected within a living cell as a consequence of its magnetic properties. Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 for suitable synthesis methods for certain of these particles. Additional materials that may be used in magnetic particles include yttrium, europium, and vanadium.

A magnetic particle may contain a magnetic material and one or more nonmagnetic materials, which may be a metal or a nonmetal. In certain embodiments of the invention, the particle is a composite particle comprising an inner core or layer containing a first material and an outer layer or shell containing a second material, wherein at least one of the materials is magnetic. Optionally both of the materials are metals. In one embodiment, the particle is an iron oxide particle, e.g., the particle has a core of iron oxide. Optionally the iron oxide is monocrystalline. In one embodiment, the particle is a superparamagnetic iron oxide particle, e.g., the particle has a core of superparamagnetic iron oxide.

Detection of magnetic particles may be performed using any method known in the art. For example, a magnetometer or a detector based on the phenomenon of magnetic resonance (NMR) can be employed. Superconducting quantum interference devices (SQUID), which use the properties of electron-pair wave coherence and Josephson junctions to detect very small magnetic fields can be used. Magnetic force microscopy or handheld magnetic readers can be used. U.S. Patent Application Publication 2003/009029 describes various suitable methods. Magnetic resonance microscopy offers one approach (Wind et al., 2000, J. Magn. Reson., 147:371).

In some embodiments, the use of magnetic particles allows for the use of a magnet to position the targeted particle in the vicinity of the target organ, tissue, and/or cell. For example, a targeted particle comprising a magnetic particle can be administered to a subject intravenously, and external magnets can be positioned so that a magnetic field is created within the body at the site of a target organ, tissue, and/or cell. The magnetic particle is then drawn to the magnetic field and retained there until the magnet is removed.

### Production of Inventive Particles

In some embodiments, inventive targeted particles comprise one or more inventive complexes and a particle. Inventive complexes generally comprise a nucleic acid targeting moiety and one or more agents to be delivered that are capable of intercalating between the base pairs of the nucleic acid targeting moiety. Inventive complexes are typically formed by incubating the therapeutic or diagnostic agent with the nucleic acid targeting moiety.

Inventive targeted particles may be manufactured using any available method. When associating inventive complexes with particles, it is desirable to have a particle which can be efficiently linked to a negatively charged nucleic acid targeting moiety using simple chemistry without adversely affecting the 3-dimensional characteristic and conformation of the nucleic acid targeting moiety. It is desirable that the targeted particle should be able to avoid uptake by the mononuclear phagocytic system after systemic administration so that it is able to reach specific organs, tissues, and/or cells in the body.

In some embodiments, the particle is associated with a second therapeutic or diagnostic agent to be delivered. In some embodiments, therapeutic or diagnostic agents are not covalently associated with a particle. To give another example, particles may comprise polymers, and therapeutic or diagnostic agents may be associated with the surface of, encapsulated within, and/or distributed throughout the polymer of an inventive particle. Agents are released by diffusion, degradation of the particle, and/or combination thereof. In some embodiments, polymers degrade by bulk erosion. In some embodiments, polymers degrade by surface erosion. In some embodiments, therapeutic or diagnostic agents are covalently associated with a particle. For such targeted particles, release and delivery of the therapeutic or diagnostic agent to a target site occurs by disrupting the association. For example, if an agent is associated with a particle by a cleavable linker, the agent is released and delivered to the target site upon cleavage of the linker.

In some embodiments, inventive complexes are physically associated with a particle. In some embodiments, physical association may be covalent. For example, the particle and complex may be directly associated with one another, e.g., by one or more covalent bonds, or may be associated by means of one or more linkers. In some embodiments, the linker forms one or more covalent or non-covalent bonds with the complex and one or more covalent or non-covalent bonds with the particle, thereby attaching them to one another. In some embodiments, a first linker forms a covalent or non-covalent bond with the complex and a second linker forms a covalent or non-covalent bond with the particle. The two linkers form one or more covalent or non-covalent bond(s) with each other.

Any suitable linker can be used in accordance with the present invention. Linkers may be used to form amide linkages, ester linkages, disulfide linkages, etc. Linkers may contain carbon atoms or heteroatoms (e.g., nitrogen, oxygen, sulfur, etc.). Typically, linkers are 1 to 50 atoms long, 1 to 40 atoms long, 1 to 25 atoms long, 1 to 20 atoms long, 1 to 15 atoms long, 1 to 10 atoms long, or 1 to 10 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

In some embodiments, a linker is an aliphatic or heteroaliphatic linker. In some embodiments, the linker is a polyalkyl linker. In certain embodiments, the linker is a polyether linker. In certain embodiments, the linker is a polyethylene linker. In certain specific embodiments, the linker is a polyethylene glycol (PEG) linker.

In some embodiments, a linker is a short peptide chain, e.g., between 1 and 10 amino acids in length, e.g., 1, 2, 3, 4, or 5 amino acids in length, a nucleic acid, an alkyl chain, etc.

In some embodiments, the linker is a cleavable linker. To give but a few examples, cleavable linkers include protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g. esterase cleavable linker), ultrasound-sensitive linkers, x-ray cleavable linkers, etc. In some embodiments, the linker is not a cleavable linker.

Any of a variety of methods can be used to associate a linker with a particle. General strategies include passive adsorption (e.g., via electrostatic interactions), multivalent chelation, high affinity non-covalent binding between members of a specific binding pair, covalent bond formation, etc. (Gao et al., 2005, Curr. Op. Biotechnol., 16:63). In some embodiments, click chemistry can be used to associate a linker with a particle (e.g. Diels-Alder reaction, Huigsen 1,3-dipolar cycloaddition, nucleophilic substitution, carbonyl chemistry, epoxidation, dihydroxylation, etc.).

A bifunctional cross-linking reagent can be employed. Such reagents contain two reactive groups, thereby providing a means of covalently associating two target groups. The reactive groups in a chemical cross-linking reagent typically belong to various classes of functional groups such as succinimidyl esters, maleimides, and pyridyldisulfides. Exemplary cross-linking agents include, e.g., carbodiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), N-Succinimidyl 3-[2-pyridyldithio]-propionamido (SPDP), succimidyl α-methylbutanoate, biotinamidohexanoyl-6-amino-hexanoic acid N-hydroxy-succinimide ester (SMCC), succinimidyl-[(N-maleimidopropionamido)-dodecaethyleneglycol]ester (NHS-PEO12), etc. For example, carbodiimide-mediated amide formation and active ester maleimide-mediated amine and sulfhydryl coupling are widely used approaches.

Common schemes for forming a targeted particle involve the coupling of an amine group on one molecule to a thiol group on a second molecule, sometimes by a two- or three-step reaction sequence. A thiol-containing molecule may be reacted with an amine-containing molecule using a heterobifunctional cross-linking reagent, e.g., a reagent containing both a succinimidyl ester and either a maleimide, a pyridyldisulfide, or an iodoacetamide. Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Polypeptides can conveniently be attached to particles via amine or thiol groups in lysine or cysteine side chains respectively, or by an N-terminal amino group. Nucleic acids such as RNAs can be synthesized with a terminal amino group. A variety of coupling reagents (e.g., succinimidyl 3-(2-pyridyldithio)propionate (SPDP) and sulfosuccinimidyl-4-(N-maleimidomethyl)cyclohexane-1-carboxylate (sulfo-SMCC) may be used to associate the various components of targeted particles. Particles can be prepared with functional groups, e.g., amine or carboxyl groups, available at the surface to facilitate association with a biomolecule. Any biomolecule can be attached to a particle and/or inventive complex using any of the methods described herein.

For additional general information on association and/or conjugation methods and cross-linkers, see the journal Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-95 Pierce Catalog, and references cited therein; Wong SS, Chemistry of Protein Conjugation and Cross-linking, CRC Press Publishers, Boca Raton, 1991; and Hermanson, G. T., Bioconjugate Techniques, Academic Press, Inc., San Diego, 1996.

In some embodiments, particles can be attached to inventive complexes directly or indirectly via non-covalent interactions. For example, particles may comprise polymers, and complexes may be associated with the surface of, encapsulated within, surrounded by, and/or distributed throughout the polymermic matrix of a particle.

Exemplary non-covalent interactions include, but are not limited to, charge interactions, affinity interactions, metal coordination, physical adsorption, host-guest interactions, hydrophobic interactions, Π stacking interactions, hydrogen bonding interactions, van der Waals interactions, magnetic interactions, electrostatic interactions, dipole-dipole interactions, etc.

In some embodiments, a particle may be associated with an inventive complex via charge interactions. For example, a particle may have a cationic surface or may be reacted with a cationic polymer, such as poly(lysine) or poly(ethylene imine), to provide a cationic surface. The particle surface can then bind via charge interactions with a negatively charged complex. One end of the nucleic acid targeting moiety is, typically, attached to a negatively charged polymer (e.g., a poly(carboxylic acid)) or an additional oligonucleotide sequence that can interact with the cationic polymer surface without disrupting the binding affinity of the nucleic acid targeting moiety for its target.

In some embodiments, a particle may be associated with an inventive complex via affinity interactions. For example, biotin may be attached to the surface of the particle and streptavidin may be attached to the complex; or conversely, biotin may be attached to the complex and the streptavidin may be attached to the surface of the particle. The biotin group and streptavidin are typically attached to the particle or to the complex via a linker, such as an alkylene linker or a polyether linker Biotin and streptavidin bind via affinity interactions, thereby binding the particle to the complex. Other specific binding pairs could be similarly used (e.g. histidine-tagged biomolecules can be associated with particles conjugated to nickel-nitrolotriaceteic acid (Ni—NTA)).

In some embodiments, a particle may be associated with an inventive complex via metal coordination. For example, a polyhistidine may be attached to one end of the nucleic acid targeting moiety, and a nitrilotriacetic acid can be attached to the surface of the particle. A metal, such as Ni²⁺, will chelate the polyhistidine and the nitrilotriacetic acid, thereby binding the complex to the particle.

In some embodiments, a particle may be associated with an inventive complex via physical adsorption. For example, a hydrophobic tail, such as polymethacrylate or an alkyl group having at least about 10 carbons, may be attached to one end of the nucleic acid targeting moiety. The hydrophobic tail will typically adsorb onto the surface of a hydrophobic particle, such as a particle comprising a polyorthoester, polysebacic anhydride, or polycaprolactone, thereby binding the complex to the particle.

In some embodiments, a particle may be associated with an inventive complex via host-guest interactions. For example, a macrocyclic host, such as cucurbituril or cyclodextrin, may be attached to the surface of the particle and a guest group, such as an alkyl group, a polyethylene glycol, or a diaminoalkyl group, may be attached to the complex; or conversely, the host group may be attached to the complex and the guest group may be attached to the surface of the particle. In some embodiments, the host and/or the guest molecule may be attached to the complex or the particle via a linker, such as an alkylene linker or a polyether linker.

In some embodiments, a particle may be associated with an inventive complex via hydrogen bonding interactions. For example, an oligonucleotide having a particular sequence may be attached to the surface of the particle, and an essentially complementary sequence may be attached to one or both ends of the complex such that it does not disrupt the binding affinity of the nucleic acid targeting moiety for its target. The nucleic acid targeting moiety will then bind to the particle via complementary base pairing with the oligonucleotide attached to the particle. Two oligonucleotides are essentially complimentary if about 80% of the nucleic acid bases on one oligonucleotide form hydrogen bonds via an oligonucleotide base pairing system, such as Watson-Crick base pairing, reverse Watson-Crick base pairing, Hoogsten base pairing, etc., with a base on the second oligonucleotide. Typically, it is desirable for an oligonucleotide sequence attached to the particle to form at least about 6 complementary base pairs with a complementary oligonucleotide attached to the nucleic acid targeting moiety.

It is to be understood that the compositions of the invention can be made in any suitable manner, and the invention is in no way limited to compositions that can be produced using the methods described herein. Selection of an appropriate method may require attention to the properties of the particular moieties being associated.

If desired, various methods may be used to separate targeted particles with an attached complex from targeted particles to which the complex has not become attached, or to separate targeted particles having different numbers of complexes attached thereto. For example, size exclusion chromatography, agarose gel electrophoresis, or filtration can be used to separate populations of targeted particles having different numbers of complexes attached thereto and/or to separate targeted particles from other entities. Some methods include size-exclusion or anion-exchange chromatography.

Any method may be used to determine whether targeted particle aggregates have formed, including measuring extinction coefficients, atomic force microscopy (AFM), etc. An extinction coefficient, generally speaking, is a measure of a substance's turbidity and/or opacity. If EM radiation can pass through a substance very easily, the substance has a low extinction coefficient. Conversely, if EM radiation hardly penetrates a substance, but rather quickly becomes “extinct” within it, the extinction coefficient is high. For example, to determine whether targeted particle aggregates have formed, EM radiation is directed toward and allowed to pass through a sample. If the sample contains primarily targeted particle aggregates, EM radiation will deflect and scatter in a pattern that is different from the pattern produced by a sample containing primarily individual targeted particles.

In general, AFM utilizes a high-resolution type of scanning probe microscope and attains resolution of fractions of an Angstrom. The microscope has a microscale cantilever with a sharp tip (probe) at its end that is used to scan a specimen surface. The cantilever is frequently silicon or silicon nitride with a tip radius of curvature on the order of nanometers. When the tip is brought into proximity of a sample surface, forces between the tip and the sample lead to a deflection of the cantilever according to Hooke's law. Typically, a feedback mechanism is employed to adjust the tip-to-sample distance to maintain a constant force between the tip and the sample. Samples are usually spread in a thin layer across a surface (e.g. mica), which is mounted on a piezoelectric tube that can move the sample in the z direction for maintaining a constant force, and the x and y directions for scanning the sample.

In general, forces that are measured in AFM may include mechanical contact force, Van der Waals forces, capillary forces, chemical bonding, electrostatic forces, magnetic forces, Casimir forces, solvation forces, etc. Typically, deflection is measured using a laser spot reflected from the top of the cantilever into an array of photodiodes. Alternatively or additionally, deflection can be measured using optical interferometry, capacitive sensing, or piezoresistive AFM probes.

### Pharmaceutical Compositions

The present invention provides novel complexes comprising one or more nucleic acid targeting moieties (e.g. aptamers or spiegelmers) and a therapeutically effective amount of one or more therapeutic or diagnostic agents that are capable of intercalating between the base pairs of the nucleic acid targeting moiety; and one or more pharmaceutically acceptable excipients. The present invention provides novel targeted particles comprising: a particle and an inventive complex; and one or more pharmaceutically acceptable excipients. In some embodiments, the present invention provides for pharmaceutical compositions comprising inventive complexes or targeted particles as described herein. Such pharmaceutical compositions may optionally comprise one or more additional therapeutically-active substances. In accordance with some embodiments, a method of administering a pharmaceutical composition comprising inventive compositions to a subject in need thereof is provided. In some embodiments, inventive compositions are administered to humans. For the purposes of the present invention, the phrase “active ingredient” generally refers to an inventive complex or targeted particle, as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions of the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

The formulations of the pharmaceutical compositions described herein may be prepared by any method known or hereafter developed in the art of pharmaceutics. In general, such preparatory methods include the step of bringing the active ingredient into association with one or more excipients and/or one or more other accessory ingredients, and then, if necessary and/or desirable, shaping and/or packaging the product into a desired single- or multi-dose unit.

A pharmaceutical composition of the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The relative amounts of the active ingredient, the pharmaceutically acceptable excipient(s), and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and perfuming agents. In certain embodiments for parenteral administration, complexes or targeted particles of the invention are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution, etc. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the active ingredient then depends upon its rate of dissolution which, in turn, may depend upon crystal size and crystalline form. In some embodiments, delayed absorption of a parenterally administered active ingredient is accomplished by dissolving or suspending the drug in an oil vehicle.

Compositions for rectal or vaginal administration are typically suppositories which can be prepared by mixing the complexes or targeted particles of this invention with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

The active ingredients can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a complex or targeted particle of this invention may include ointments, pastes, creams, lotions, gels, powders, solutions, sprays, inhalants and/or patches. Generally, the active component is admixed under sterile conditions with a pharmaceutically acceptable excipient and/or any needed preservatives and/or buffers as may be required. Additionally, the present invention contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the excipients and/or additional ingredients described herein.

A pharmaceutical composition of the invention may be prepared, packaged, and/or sold in a formulation suitable for pulmonary administration via the buccal cavity. Such a formulation may comprise dry particles which comprise the active ingredient and which have a diameter in the range from about 0.5 μm to about 7 μm or from about 1 μm to about 6 μm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 μm and at least 95% of the particles by number have a diameter less than 7 μm. Alternatively, at least 95% of the particles by weight have a diameter greater than 1 μm and at least 90% of the particles by number have a diameter less than 6 μm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 μm to about 200 μm.

The formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may comprise one or more of the excipients and/or additional ingredients described herein. A pharmaceutical composition of the invention may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the excipients and/or additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 μm to about 200 μm, and may further comprise one or more of the excipients and/or additional ingredients described herein.

A pharmaceutical composition of the invention may be prepared, packaged, and/or sold in a formulation suitable for ophthalmic administration. Such formulations may, for example, be in the form of eye drops including, for example, a 0.1%/1.0% (w/w) solution and/or suspension of the active ingredient in an aqueous or oily liquid excipient. Such drops may further comprise buffering agents, salts, and/or one or more other of the excipients and/or additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005.

Administration

In some embodiments, a therapeutically effective amount of an inventive composition is delivered to a patient and/or organism prior to, simultaneously with, and/or after diagnosis with a disease, disorder, and/or condition (e.g. cancer). In some embodiments, a therapeutic or diagnostic amount of an inventive composition is delivered to a patient and/or organism prior to, simultaneously with, and/or after onset of symptoms of a disease, disorder, and/or condition. In some embodiments, the amount of inventive complex or targeted particle is sufficient to treat, alleviate, ameliorate, relieve, delay onset of, inhibit progression of, reduce severity of, and/or reduce incidence of one or more symptoms or features of the disease, disorder, and/or condition.

The compositions, according to the method of the present invention, may be administered using any amount and any route of administration effective for treatment. The exact amount required will vary from subject to subject, depending on the species, age, and general condition of the subject, the severity of the infection, the particular composition, its mode of administration, its mode of activity, and the like. The compositions of the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The pharmaceutical compositions of the present invention may be administered by any route. In some embodiments, the pharmaceutical compositions of the present invention are administered by a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), transdermal, mucosal, nasal, buccal, enteral, sublingual; by intratracheal instillation, bronchial instillation, and/or inhalation; and/or as an oral spray, nasal spray, and/or aerosol. Specifically contemplated routes are systemic intravenous injection, regional administration via blood and/or lymph supply, and/or direct administration to an affected site. In some embodiments, inventive complexes or targeted particles are administered parenterally. In some embodiments, inventive complexes or targeted particles are administered intravenously. In some embodiments, inventive complexes or targeted particles are administered orally.

In some embodiments, inventive complexes or targeted particles are administered directly to an affected site. For example, inventive complexes or targeted particles may be administered locally near a tumor and/or may be administered directly to a tumor. In some embodiments, local administration refers to administration of complexes or targeted particles directly to a specific organ (e.g. injection into the prostate, in the case of prostate cancer). In some embodiments, local administration refers to administration of complexes or targeted particles directly to a particular organ, tissue, and/or cell. Local administration may be achieved via injection of complexes or targeted particles directly into a tumor or in the vicinity of a tumor. Local administration may be achieved by topical administration of complexes or targeted particles at or near the site of a tumor. Local administration may be achieved by implantation of complexes or targeted particles at or near a site of a tumor by stereotactic surgery. Local administration may be achieved by implantation of complexes or targeted particles at or near the site of a tumor during surgical removal of the tumor. In some embodiments, local administration refers to administration of complexes or targeted particles to a specific cell or population of cells (e.g. prostate cancer cells).

In general the most appropriate route of administration will depend upon a variety of factors including the nature of the agent (e.g., its stability in the environment of the gastrointestinal tract), the condition of the subject (e.g., whether the subject is able to tolerate oral administration), etc. However, the invention encompasses the delivery of the inventive pharmaceutical composition by any appropriate route taking into consideration likely advances in the sciences of drug delivery.

In certain embodiments, complexes or targeted particles of the invention may be administered at therapeutic agent in amounts ranging from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Combination Therapy

In some embodiments, the present invention encompasses “therapeutic cocktails” comprising inventive complexes or targeted particles. In some embodiments, complexes or targeted particles comprise a single species of nucleic acid targeting moiety which can bind to multiple targets. In some embodiments, different complexes or targeted particles comprise different nucleic acid targeting moiety species, and all of the different nucleic acid targeting moiety species can bind to the same target. In some embodiments, different complexes or targeted particles comprise different nucleic acid targeting moiety species, and all of the different nucleic acid targeting moiety species can bind to different targets. In some embodiments, such different targets may be associated with the same cell type. In some embodiments, such different targets may be associated with different cell types.

It will be appreciated that complexes or targeted particles and pharmaceutical compositions of the present invention can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, an inventive complex or targeted particle useful for detecting tumors may be administered concurrently with another agent useful for detecting tumors), or they may achieve different effects (e.g., control of any adverse effects).

Pharmaceutical compositions of the present invention may be administered either alone or in combination with one or more other therapeutic agents. By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a time schedule determined for that agent. Additionally, the invention encompasses the delivery of inventive pharmaceutical compositions in combination with agents that may improve their bioavailability, reduce and/or modify their metabolism, inhibit their excretion, and/or modify their distribution within the body.

The particular combination of therapies (therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, an inventive complex or targeted particle may be administered concurrently with another therapeutic agent used to treat the same disorder), and/or they may achieve different effects (e.g., control of any adverse effects). In some embodiments, complexes or targeted particles of the invention are administered with a second therapeutic agent that is approved by the U.S. Food and Drug Administration.

In will further be appreciated that therapeutically active agents utilized in combination may be administered together in a single composition or administered separately in different compositions.

In general, it is expected that agents utilized in combination with be utilized at levels that do not exceed the levels at which they are utilized individually. In some embodiments, the levels utilized in combination will be lower than those utilized individually.

In some embodiments, inventive compositions may be administered in combination with any therapeutic agent or therapeutic regimen that is useful to treat, alleviate, ameliorate, relieve, delay onset of, inhibit progression of, reduce severity of, and/or reduce incidence of one or more symptoms or features of cancer. For example, inventive compositions may be administered in combination with traditional cancer therapies including, but not limited to, surgery, chemotherapy, radiation therapy, hormonal therapy, immunotherapy, complementary or alternative therapy, and any combination of these therapies.

In some embodiments, inventive compositions are administered in combination with surgery to remove a tumor. Because complete removal of a tumor with minimal or no damage to the rest of a patient's body is typically the goal of cancer treatment, surgery is often performed to physically remove part or all of a tumor. If surgery is unable to completely remove a tumor, additional therapies (e.g. chemotherapy, radiation therapy, hormonal therapy, immunotherapy, complementary or alternative therapy) may be employed.

In some embodiments, inventive compositions are administered in combination with radiation therapy. Radiation therapy (also known as radiotherapy, X-ray therapy, or irradiation) is the use of ionizing radiation to kill cancer cells and shrink tumors. Radiation therapy may be used to treat almost any type of solid tumor, including cancers of the brain, breast, cervix, larynx, lung, pancreas, prostate, skin, stomach, uterus, or soft tissue sarcomas. Radiation can be used to treat leukemia and lymphoma. Radiation therapy can be administered externally via external beam radiotherapy (EBRT) or internally via brachytherapy. Typically, the effects of radiation therapy are localized and confined to the region being treated. Radiation therapy injures or destroys tumor cells in an area being treated (e.g. a target organ, tissue, and/or cell) by damaging their genetic material, preventing tumor cells from growing and dividing. In general, radiation therapy attempts to damage as many tumor cells as possible while limiting harm to nearby healthy organs, tissues, and/or cells. Hence, it is often administered in multiple doses, allowing healthy organs, tissues, and/or cells to recover between fractions.

In some embodiments, inventive compositions are administered in combination with immunotherapy. Immunotherapy is the use of immune mechanisms against tumors which can be used in various forms of cancer, such as breast cancer (e.g. trastuzumab/Herceptin®), leukemia (e.g. gemtuzumab ozogamicin/Mylotarg®), and non-Hodgkin's lymphoma (e.g. rituximab/Rituxan®). In some embodiments, immunotherapy agents are monoclonal antibodies directed against proteins that are characteristic to the cells of the cancer in question. In some embodiments, immunotherapy agents are cytokines that modulate the immune system's response. In some embodiments, immunotherapy agents may be vaccines.

In some embodiments, vaccines can be administered to prevent and/or delay the onset of cancer. In some embodiments, cancer vaccines prevent and/or delay the onset of cancer by preventing infection by oncogenic infectious agents. In some embodiments, cancer vaccines prevent and/or delay the onset of cancer by mounting an immune response against cancer-specific epitopes. To give but one example of a cancer vaccine, an experimental vaccine for HPV types 16 and 18 was shown to be 100% successful at preventing infection with these types of HPV and, thus, are able to prevent the majority of cervical cancer cases (Harper et al., 2004, Lancet, 364:1757).

In some embodiments, inventive compositions are administered in combination with complementary and alternative medicine treatments. Some exemplary complementary measures include, but are not limited to, botanical medicine (e.g. use of mistletoe extract combined with traditional chemotherapy for the treatment of solid tumors); acupuncture for managing chemotherapy-associated nausea and vomiting and in controlling pain associated with surgery; prayer; psychological approaches (e.g. “imaging” or meditation) to aid in pain relief or improve mood. Some exemplary alternative measures include, but are not limited to, diet and other lifestyle changes (e.g. plant-based diet, the grape diet, and the cabbage diet).

In some embodiments, a therapeutic or diagnostic agent to be delivered that is capable of intercalating between the base pairs of the nucleic acid targeting moiety can be associated with unpleasant, uncomfortable, and/or dangerous side effects. For example, chronic pain often results from continued tissue damage due to the cancer itself or due to the treatment (i.e., surgery, radiation, chemotherapy). Alternatively or additionally, such therapies are often associated with hair loss, nausea, vomiting, diarrhea, constipation, anemia, malnutrition, depression of immune system, infection, sepsis, hemorrhage, secondary neoplasms, cardiotoxicity, hepatotoxicity, nephrotoxicity, ototoxicity, etc. Thus, inventive compositions which are administered in combination with any of the traditional cancer treatments described herein may be also be administered in combination with any therapeutic agent or therapeutic regimen that is useful to treat, alleviate, ameliorate, relieve, delay onset of, inhibit progression of, reduce severity of, and/or reduce incidence of one or more side effects of cancer treatment. To give but a few examples, pain can be treated with opioids and/or analgesics (e.g. morphine, oxycodone, antiemetics, etc.); nausea and vomiting can be treated with 5-HT₃ inhibitors (e.g. dolasetron/Anzemet®, granisetron/Kytril®, ondansetron/Zofran®, palonsetron/Aloxi®) and/or substance P inhibitors (e.g. aprepitant/Emend®); immunosuppression can be treated with a blood transfusion; infection and/or sepsis can be treated with antibiotics (e.g. penicillins, tetracyclines, cephalosporins, sulfonamides, aminoglycosides, etc.); and so forth.

In addition to the complexes or targeted particles described above that are useful for simultaneously diagnosing and treating cancer, in some embodiments, inventive compositions may be administered and/or inventive diagnostic methods may be performed in combination with (e.g. in parallel with) any therapeutic or diagnostic agent or regimen that is useful to diagnose one or more symptoms or features of cancer (e.g. detect the presence of and/or locate a tumor). In some embodiments, inventive complexes or targeted particles may be used in combination with one or more other diagnostic agents. To give but one example, complexes or targeted particles used to detect tumors may be administered in combination with other agents useful in the detection of tumors. For example, inventive complexes or targeted particles may be administered in combination with traditional tissue biopsy followed by immunohistochemical staining and serological tests (e.g. prostate serum antigen test). Alternatively or additionally, inventive complexes or targeted particles may be administered in combination with a contrasting agent for use in computed tomography (CT) scans and/or MRI.

### Kits

The invention provides a variety of kits comprising one or more of the complexes or targeted particles of the invention. For example, the invention provides a kit comprising an inventive complex or targeted particle and instructions for use. A kit may comprise multiple different complexes and/or targeted particles. A kit may comprise any of a number of additional components or reagents in any combination (e.g. pharmaceutically acceptable excipients). All of the various combinations are not set forth explicitly but each combination is included in the scope of the invention.

According to certain embodiments of the invention, a kit may include, for example, (i) a complex comprising a nucleic acid targeting moiety and one or more therapeutic or diagnostic agents to be delivered which are capable of intercalating between the base pairs of the nucleic acid targeting moiety; (ii) instructions for administering the complex to a subject in need thereof.

In some embodiments, a kit may include, for example, (i) a targeted particle comprising a particle, a specific nucleic acid targeting moiety, and one or more particular therapeutic or diagnostic agents to be delivered which are capable of intercalating between the base pairs of the nucleic acid targeting moiety; (ii) instructions for administering the targeted particle to a subject in need thereof.

Kits typically include instructions for use of inventive complexes or targeted particles. Instructions may, for example, comprise protocols and/or describe conditions for production of complexes or targeted particles, administration of complexes or targeted particles to a subject in need thereof, design of novel complexes or targeted particles, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### EXEMPLIFICATION

### Example 1

### Aptamer-Doxorubicin Physical Conjugate as a Novel Targeted Drug Delivery Platform

### Materials and Methods

Formation of Aptamer-Dox Complexes

A complex comprising the A10 PSMA aptamer (RNA-Tec, Belgium) and doxorubicin (Dox) was generated through the stepwise addition of increasing molar ratio of aptamer to a fixed concentration of doxorubicin (3 μM) in the presence of 0.1 M sodium acetate, 0.05 M sodium chloride, and 0.01 M magnesium chloride. The fluorescence of Dox was measured at 35 minutes by exciting the solution at 480 nm and recording the emission in the interval of 500 nm-720 nm (1.5 mm slit) on a Shimadzu RF-PC100 spectrofluorophotometer.

Release of Dox from Aptamer-Dox Complexes

Aptamer-doxorubicin complexes [(1:1.2 mole ratio), doxorubicin concentration 40 μM were generated and size fractionated through NAP 5 (G25-DNA grade BIORAD column) to remove free unbound Dox in solution. The resulting complex solution (1 mL) was transferred to dialysis vials (3.5 kDa cut off, PIERCE) and dialyzed against buffer (0.1 M sodium acetate, 0.05 M sodium chloride, and 0.01 M magnesium chloride) at ambient temperature. At selected time intervals, buffer solution outside the dialysis vials was taken for UV-VIS analysis and replaced with fresh buffer solution. Free Dox (40 μM) was also dialyzed under same condition as control. Dox concentration was calculated based on the absorbance intensity at 485 nm.

Assessment of Cell Uptake by Confocal Laser Scanning Microscopy

Prostrate cancer cell lines LNCaP(PSMA+) and PC3 (PSMA−) (5*103 cells/mL) were grown in chamber slides in RPMI 1640 media with 10% fetal bovine serum for 24 hours to attain 70% confluence. Before incubation with complex, cells were pre-incubated with OPTIMEM media for 30 minutes, followed by incubation with physical complex of 1.0:1.1 molar ratio of Dox:aptamer with a doxorubicin concentration of 1.5 μM for 2 hours, the cells were washed with PBS twice, fixed with 3.5% HCHO for 10 minutes, washed, mounted with Vector mounting media and cover slipped. Fluorescence images were obtained (Carl Zeiss LSM 510, Ar laser 488, long pass filter 560,63× water lens.)

Flow Cytometry

Cellular uptake of the complex was confirmed using flow cytometry (EPICS XL Flow cytometry systems, Beckman Coulter, Inc). Briefly, 5×104 cells were seeded onto 12 well plates (n=4) for 24 hours followed by incubation for 2 hours with the complex solution (1.0:1.1 mole ratio of Dox:aptamer) such that final Dox concentration was 1.5 μM. Cells were washed with PBS twice, trypsinized, centrifuged at 1000 rpm for 3 minutes and resuspended in PBS for FACS analysis. Data were processed with EXPO 32 software.

MTT Cell Viability Assay

MTT assays were performed essentially as previously described (Akaishi et al., 1995, Tohoku J. Exp. Med., 175:29). Briefly, 100 μl aliquot of LNCaP or PC3 cells (5*103 cells/mL) were seeded in 96 well plates (n=5) and allowed to grow overnight and treated with 100 μL of either (1) aptamer-Dox complex (1.0:1.1 Dox:aptamer molar ratio; doxorubicin concentration of 5 μM); (2) Dox alone (5 μM); or (3) aptamer alone (3.8 μM) for 2 hours, washed, and further incubated in fresh media for a total of 24 hours. Cells were next washed twice with PBS and treated with 20 μL MTT solution, which was aspirated. 100 μL DMSO was added, mixed, and the absorbance was measured with a microplate reader at 570 nm.

Aptamer Cell Binding Assay

Briefly, (3*104/100 μA) LNCaP cells were taken and fixed in suspension with 4% formaldehyde and washed with PBS. Cells were incubated with saturating aptamer concentration (100 nM) and with aptamer-Dox complex (1.0:1.5 molar ratio) for 30 minutes. Cells were pelleted, supernatant was removed, and cells were washed with PBS. Finally, bound aptamer and aptamer-Dox complexes were recovered by treating cells with preheated (65° C.) elution buffer (100 nM sodium citrate, 7 M urea, 10 mM EDTA) and washed over 3K spin filter twice and resuspended in 100 μl DNase RNase free water. A 5 μl aliquot each of free aptamer, bound aptamer, and bound aptamer-Dox complex was taken and subjected to RT-PCR amplification. The product was loaded on 1.5% agarose gel, and the intensity of the bands was resolved using densitometry.

### Results

Production of Aptamer-Anthracycline Complexes

The two-dimensional structure of the A10 PSMA (Lupold et al., 2002, Cancer Res., 62:4029) aptamer used herein was predicted by the M fold program (Zuker, 2003, Nuc. Acid. Res., 31:3406). The anthracycline class of drugs, including Doxorubicin (Dox), has fluorescence properties that can become quenched after intercalation into DNA (Haj et al., 2003, Chem. Biol. Interact., 145:349; and Valentini et al., 1985, Farmaco [Sci], 40:377). The present invention encompasses the recognition that Dox can intercalate within an RNA aptamer.

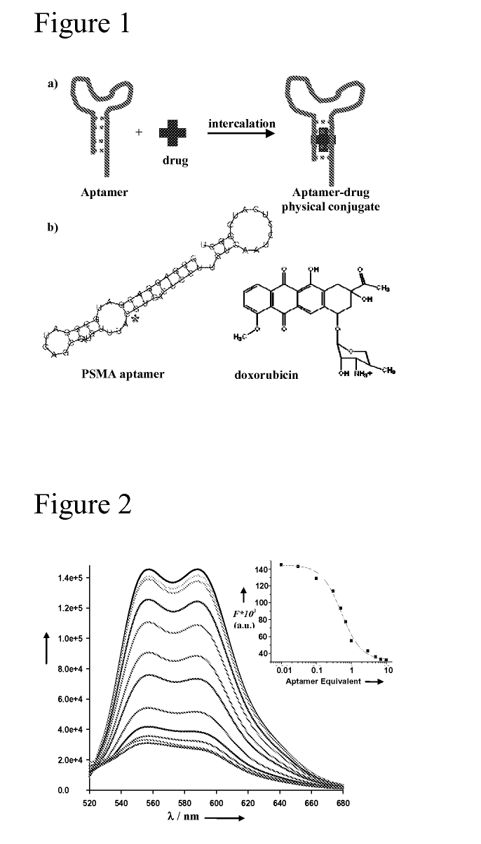

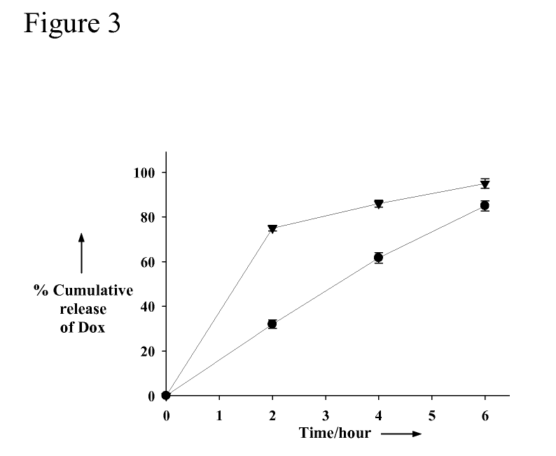

To examine whether such intercalation occurs within an RNA aptamer, binding studies were carried out between the A10 PSMA aptamer and Dox. Fluorescence spectroscopy was used to examine the association of Dox with the A10 PSMA aptamer. Sequential decreases in the native fluorescence spectrum of Dox were observed when a fixed concentration of Dox was incubated with an increasing molar ratio of the A10 PSMA aptamer, results consistent with the intercalation of Dox within the A10 PSMA aptamer (FIG. 2). Dox preferentially binds to double-stranded 5′-GC-3′ or 5′-CG-3′ sequences (Chaires et al., 1990, Biochemistry, 29:6145; and Frederick et al., 1990, Biochemistry, 29:2538), and evaluation of the predicted A10 aptamer secondary structure reveals one possible site for Dox intercalation, as marked by an asterisk in FIG. 1B. Incubation of Dox with the A10 aptamer results in very effective quenching of the Dox fluorescence at approximately 1:1.2 molar equivalence of Dox to aptamer, suggesting that Dox associates with the A10 aptamer by intercalating into its predicted CG sequence (FIG. 2). The inset of FIG. 2 shows a Hill plot of fluorescence quenching as a function of increasing aptamer concentration. The dissociation constant (K_{d}=600 nm) of the aptamer-Dox complex was derived from this figure and suggests a spontaneously-formed stable physical association. The stability of the aptamer-Dox complex was further confirmed by high-pressure liquid chromatography (HPLC) where the complex peak appeared at a different elution time from those of the native aptamer and Dox. A study of the release of Dox from the aptamer-Dox complex over time was conducted by using a dialysis tube (FIG. 3). Upon dialysis, more than 80% Dox release was observed in 6 hours with zero order kinetics, suggesting that Dox is released from the complex beyond the concentration of its dissociation constant by simple diffusion.

In Vitro Binding and Uptake of Apt-Dox Complexes

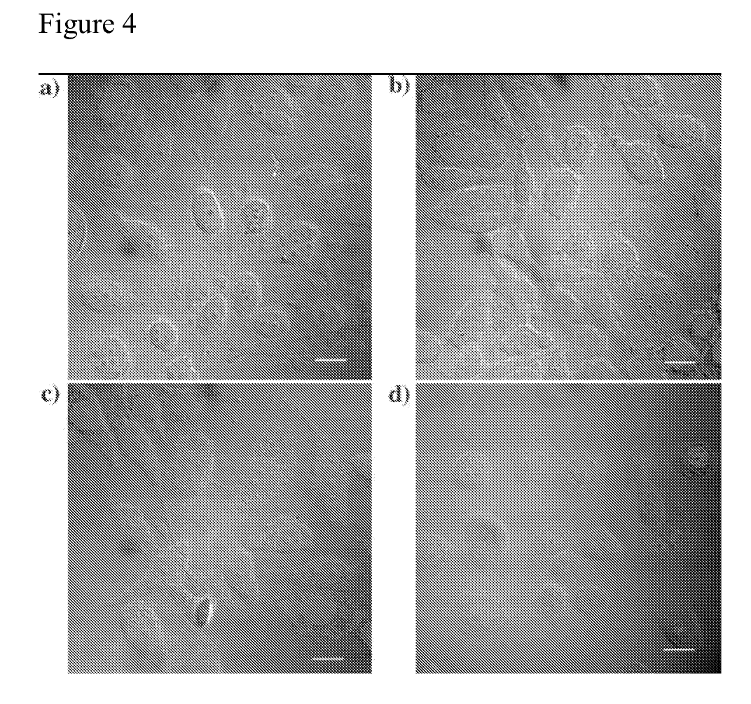

To evaluate the feasibility of the aptamer-Dox physical conjugate as a targeted drug-delivery platform, in vitro binding and uptake studies were performed using LNCaP prostate epithelial cells which express the target PSMA protein on their plasma membranes. The PC3 prostate epithelial cells which do not express any detectable level of the PSMA protein were used as a negative control (FIG. 4). Confocal laser scanning microscopy data demonstrate that while free Dox readily diffuses through the plasma membrane of LNCaP and PC3 cells with equal efficiency (FIGS. 4A and 4B), there is a remarkable specificity in the uptake of the aptamer-Dox conjugate by LNCaP, but not PC3 cells (FIGS. 4C and 4D), as marked by strong nuclear fluorescence that is consistent with the intercalation of Dox within genomic DNA.

The mechanisms of uptake of aptamer-Dox complex and the free Dox by LNCaP cells appear distinct (Yoo et al., 2000, J. Control. Release, 68:419). Unlike free Dox, which almost exclusively stains nuclei, aptamer-Dox complexes demonstrate both nuclear and cytosolic staining, with the latter predominately in the form of punctuate granules that are consistent with compartmentalization of the Dox within endosomes (FIG. 4C). This pattern of cytosolic staining is consistent with receptor-mediated endocytic uptake of aptamer-Dox complexes, which results after binding of the conjugate to the PSMA protein on the LNCaP plasma membrane. Aptamer-Dox complexes failed to produce any cytosolic staining of PC3 cells, a result consistent with the lack of PSMA protein expression in these cells (FIG. 4D). Without wishing to be bound by any one theory, the weak fluorescence staining of the PC3 nuclei after incubating with the aptamer-Dox complex is likely attributable to a small amount of free Dox that may be present in the media bathing the cells. Indeed, the LNCaP— and PC3-binding data suggest that the majority of Dox remains in the form of a complex with the aptamer, thereby demonstrating the stability of the aptamer-Dox complex over time in the culture media. Furthermore, the data demonstrate the ability of an aptamer to retain its binding characteristics while the Dox is intercalated within it, so allowing the targeted delivery of Dox to the cells that express the aptamer target.

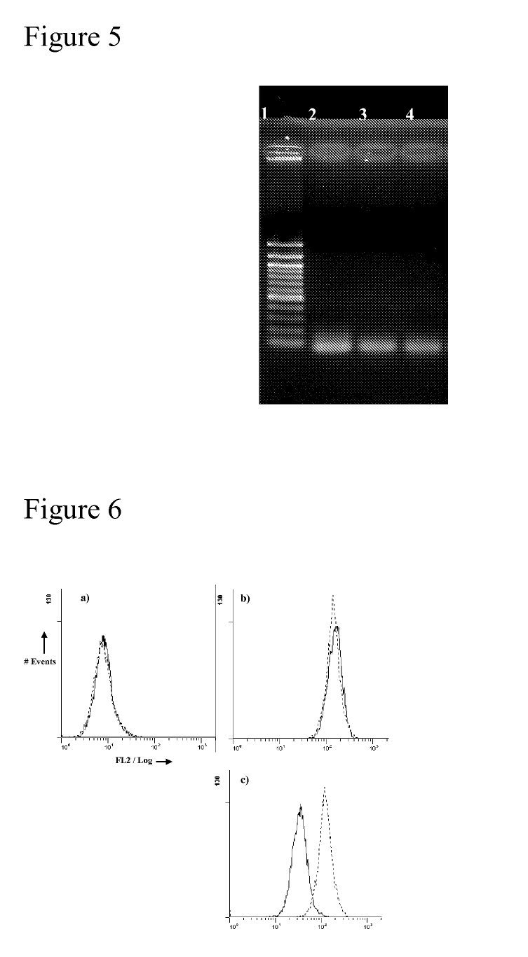

Next, the binding characteristics of an equimolar concentration of the aptamer-Dox complex were compared to the free aptamer in LNCaP binding assays (Chu et al., 2006, Biosens. Bioelectron., 21:1859). By using quantitative PCR amplification of LNCaP-bound aptamers, about 84% of the binding ability of the A10 aptamer was shown to be retained by the aptamer-Dox complex (FIG. 5). This result also demonstrates the ability of an aptamer to retain its binding characteristics while the Dox is intercalated within it, thereby allowing the targeted delivery of Dox to the cells that express the aptamer target.

The targeting specificity of the aptamer-Dox physical conjugate was next quantified by flow cytometry experiments (FIG. 6). The data demonstrate near-identical staining of LNCaP and PC3 cells after treatment of these cells with free Dox (FIGS. 6A and 6B). However, when LNCaP and PC3 cells were incubated with the aptamer-Dox complex, there was a significant enhancement in the fluorescence signal from LNCaP cells as compared to that from PC3 cells (FL2 log intensity for LNCaP was 123±4.66 versus 35±1.79 for PC3; mean±standard error (SE), number of samples (N)=4), which validates the targeting specificity of the aptamer-Dox physical conjugate (FIG. 6C). Taken together, the microscopy and flow cytometry data demonstrate the proof of concept for the feasibility of the aptamer-Dox physical conjugate to serve as a novel drug-delivery platform for a variety of applications in oncology.

Cytotoxicity of Aptamer-Dox Complexes

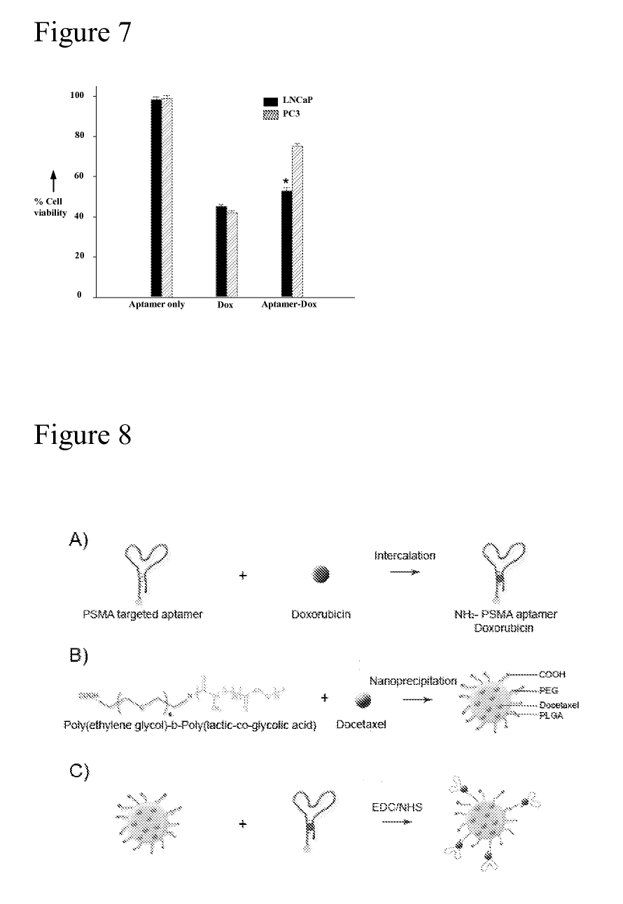

Next, it was determined whether the targeted delivery of the aptamer-Dox physical conjugate to LNCaP cells results in enhanced cellular cytotoxicity (Akaishi et al., 1995, J. Exp. Med., 175:29) compared to that in PC3 control cells. First, it was demonstrated in escalating-dose studies that the cytotoxicity of free Dox to LNCaP and PC3 cells is equipotent. At a dose where the cytotoxicity of free Dox had reached a plateau near its maximum, the cytotoxic efficacy of free Dox (5 μm) was compared to that of the aptamer-Dox complex (5 μm), as well as that of free aptamer without Dox, on LNCaP and PC3 cells by MTT assay. The data demonstrate that while the cytotoxicity of free Dox is equipotent against LNCaP and PC3 cells, the cytotoxicity of the aptamer-Dox complex is significantly enhanced against the targeted LNCaP cells as compared to the nontargeted PC3 cells (cellular viability: 52.8%±1.73 LNCaP versus 75.2%±1.19 PC3; mean±SE, N=5; probability value (p)<0.005; FIG. 7). The data demonstrate a near-equipotent cytotoxicity of the aptamer-Dox complex to the LNCaP cells as compared to that of free Dox. The free aptamer without Dox had no inherent cytotoxicity to LNCaP or PC3 cells (FIG. 7). Without wishing to be bound by any one theory, the data suggest that, after endocytic uptake, the aptamer-Dox complex releases the Dox molecules inside the LNCaP cells, possibly due to the aptamer-Dox dissociation constant favoring the release of Dox because of the relatively negligible concentrations of Dox inside the cells. Alternatively, the release of Dox from the aptamer-Dox complex may occur through gradual degradation of the aptamer by endonucleases in the lysosomes after cellular uptake. It is possible that a combination of these factors may contribute to the observed findings. In contrast to the significant cytotoxic effects of the aptamer-Dox complex to the LNCaP cells, the cytotoxicity of the complex to PC3 cells was significantly less pronounced, a result consistent with the lack of PSMA expression in PC3 cells.

### DISCUSSION

In conclusion, by exploiting the ability of anthracycline drugs to intercalate between bases of polynucleotides, a novel complex was made comprising the anticancer drug Dox and the A10 RNA aptamer that binds to the PSMA protein on the surface of prostate cancer cells. The stability and efficacy of this conjugate to serve as a novel drug-delivery platform was further demonstrated in vitro. The data suggest that the aptamer-Dox physical conjugate is stable in the cell culture medium and could differentially and with high efficiency target the PSMA-expressing LNCaP cells. The specificity of the system was further demonstrated by the lack of targeting of the PSMA-negative PC3 cells. Without wishing to be bound by any one theory, the inventors expect that the small size of the aptamer-Dox physical-conjugate system (approximately 18 kD) as compared to that of similar antibody-based immunoconjugates (approximately 150 kD) may facilitate the rapid vascular extravasation and intratumoral penetration of the former, thereby making it a therapeutically effective drug delivery system for in vivo applications (Wu, 2005, Nat. Biotechnol., 23:1137).

Furthermore, these systems may be combined such that a targeted nanoparticle-aptamer targeted particle system may deliver distinct drugs through encapsulation within the nanoparticles and through intercalation within the aptamers, with the result of a temporally distinct release of two or more drugs for combination chemotherapy (Farokhzad et al., 2006, Proc. Natl. Acad. Sci., USA, 103:6315; Farokhzad et al., 2004, Cancer Res., 64:7668; and Farokhzad et al., 2006, Expert Opin. Drug Delivery, 3:311). The inventors anticipate that the aforementioned aptamer-drug platform technology based on the intercalation of anthracyclines within the bases of aptamers may be utilized in distinct ways to develop novel targeted therapeutic modalities for more effective cancer chemotherapy, such as the methods and systems described in Examples 2 and 3.

### Example 2

### Co-Delivery of Hydrophobic and Hydrophilic Drugs from Nanoparticle-Aptamer Targeted Particles

### Materials and Methods

Materials

Docetaxel (Dxtl), Doxorubicin (Dox), and ¹⁴C-paclitaxel were purchased from Sigma-Aldrich (St. Louis, Mo.). Poly(D,L-lactide-co-glycolide) (50/50) with terminal carboxylate groups (PLGA, inherent viscosity 0.20 dl/g in hexafluoroisopropanol, MW approximately 17 kDa) was obtained from Absorbable Polymers International (Pelham, Ala.). NH₂—PEG-COOH (MW 3400) was purchased from Nektar Therapeutics (San Carlos, Calif.). All reagents were analytical grade or above and used as received, unless otherwise stated. Molecular biology buffers were purchased from Boston BioProducts (Worcester, Mass.). Tissue culture reagents and the LNCaP cell line were obtained from American Type Culture Collection (Manassas, Va.). RNA aptamer (sequence: 5′—NH₂-spacer-[GGG/AGG/ACG/AUG/CGG/AUC/AGC/CAU/GUU/UAC/GUC/ACU/CCU/UGU/CAA/UCC/UCA/UCG/GCiT-3′(SEQ ID NO.: 3)] with 2′-fluoro pyrimidines, a 5′-amino group attached by a hexaethyleneglycol spacer and a 3′-inverted T cap) was custom synthesized by RNA-TEC (Leuven, Belgium) at a purity above 90%.

Synthesis of PLGA-b-PEG Copolymers

Carboxylate-functionalized copolymer PLGA-b-PEG was synthesized by the attachment of COOH-PEG-NH₂ to PLGA-COOH. PLGA-COOH (5 g, 0.28 mmol) in methylene chloride (10 ml) was converted to PLGA-NHS with excess N-hydroxysuccinimide (NHS, 135 mg, 1.1 mmol) in the presence of 1-ethyl-3-(3-dimethylaminopropyl)-carbodiimide (EDC, 230 mg, 1.2 mmol). PLGA-NHS was precipitated with ethyl ether (5 ml), and repeatedly washed in an ice-cold mixture of ethyl ether and methanol to remove residual NHS. After drying under vacuum, PLGA-NHS (1 g, 0.059 mmol) was dissolved in chloroform (4 ml) followed by addition of NH₂—PEG-COOH (250 mg, 0.074 mmol) and N,N-diisopropylethylamine (28 mg, 0.22 mmol). The co-polymer was precipitated with cold methanol after 12 hours and washed with the same solvent (3×5 ml) to remove unreacted PEG. The resulting PLGA-PEG block co-polymer was dried under vacuum and used for nanoparticle (NP) preparation without further treatment. ¹H NMR (CDCl₃ at 300 Hz) δ 5.2 (m, ((OCH(CH₃)C(O)OCH₂C(O))ₙ—(CH₂CH₂O)ₘ), 4.8 (m, ((OCH(CH₃)C(O)OCH₂C(O))ₙ—(CH₂CH₂O)ₘ), 3.7 (s, ((OCH(CH₃)C(O)OCH₂C(O))ₙ—(CH₂CH₂O)ₘ), 1.6 (d, ((OCH(CH₃)C(O)OCH₂C(O))ₙ—(CH₂CH₂O)ₘ).

Formulation of NP(Dtxl)-Apt(Dox) Targeted Particles

PLGA-b-PEG NPs were prepared by using the nanoprecipitation method. Briefly, PLGA-PEG-COOH (10 mg/ml) and Dtxl (0.5 mg/ml) were dissolved in acetonitrile and together mixed dropwise into water, giving a final polymer concentration of 3.3 mg/ml. NPs were stirred for 1 hour, and the remaining organic solvent was removed using a rotary evaporator at reduced pressure. NPs were centrifuged at 10,000 g for 15 minutes and washed with deionized water, and the size (in nanometers) and surface charge (zeta potential in millivolts) of NPs were evaluated by Quasi-elastic laser light scattering (QELS) by using a ZetaPALS dynamic light-scattering detector (15 mW laser, incident beam=676 nm; Brookhaven Instruments, Holtsville, N.Y.). Separately, a physical conjugate between the A10 PSMA aptamer (RNA-Tec, Belgium) and Dox was generated through the stepwise addition of increasing molar ratio of aptamer to a fixed concentration of Dox (3 μM) in the presence of 0.1 M sodium acetate, 0.05 M sodium chloride, and 0.01 M magnesium chloride. The fluorescence of Dox was measured at 35 minutes by exciting the solution at 480 nm and recording the emission in the interval of 500-720 nm (1.5 mm slit) on a Shimadzu RF-PC100 spectrofluorophotometer. Next, the Apt-Dox conjugate was filtered through NAP 5 (G25-DNA grade BIORAD column) to remove the free unbound Dox in solution. To conjugate Apt(Dox) to the NP(Dtxl) surface, the PLGA-PEG-COOH NP suspension (10 μg/μL in DNase RNase-free water) was incubated with 400 mM 1-ethyl-3-(3-dimethylaminopropyl)-carbodiimide and 100 mM N-hydroxysuccinimide for 15 minutes at room temperature with gentle stirring. The resulting N-hydroxysuccinimide-activated particles were covalently linked to 5′-NH2 modified A10 PSMA Apts (2% weight compared with polymer concentration). The resulting NP-Apt targeted particles were washed, resuspended in PBS, and used immediately.

HPLC Measurements

The release of Dtxl and Dox from targeted nanoparticle-aptamer targeted particles [NP(Dtxl)-Apt(Dox)] was performed in PBS buffer at 37° C. using a Slide-A-Lyzer MINI dialysis microtube with a molecular weight cut-off of 3,500 kD (Pierce, Rockford, Ill.). To measure the drug release profile of Dtxl, 3 ml of NP(Dtxl)-Apt(Dox) in PBS (10 mg/ml) were split equally into 30 MINI dialysis microtubes (100 μA per microtube). These microtubes were dialyzed in 4 L PBS buffer at 37° C. with gentle stirring. At each data point, NP(Dtxl)-Apt(Dox) solutions from three microtubes were collected separately and mixed with an equal volume of acetonitrile to dissolve the nanoparticles. The resulting free Dtxl content in each microtube was assayed using an Agilent (Palo Alto, Calif.) 1100 HPLC equipped with a pentafluorophenyl column (Curosil-PFP, 250×4.6 mm, 5 g; Phenomenex, Torrance, Calif.). Dtxl absorbance was measured by a UV-vis detector at 227 nm and a retention time of 12 minutes in 1 ml/min 50/50 acetonitrile/water mobile phase. To measure the release of Dox, 300 μl of the same NP(Dtxl)-Apt(Dox) solution was equally distributed into three MINI dialysis microtubes. These microtubes were each dialyzed in 1 ml PBS buffer at 37° C. with gentle stirring. At each data point, 100 μl samples from each dialysate were gathered, replaced by the same amount of fresh PBS buffer, and then assayed by HPLC with a UV-vis detector at a wavelength of 490 nm and a retention time of 3 minutes in 1 ml/min 40/60 acetonitrile/water mobile phase.

Fluorescence Microscopy Measurements

To visualize cell uptake of drugs using fluorescence microscopy, a hydrophobic fluorescent dye, NBD-cholesterol (22-(N-(7-nitrobenz-2-oxa-1,3-diazol-4-yl)amino)-23,24-bisnor-5-cholen-3β-ol, Invitrogen, Carlsbad, Calif.), was encapsulated inside PLGA-b-PEG nanoparticles as an analog of a hydrophobic drug. The fluorescence emission spectrum (excitation/emission=460 nm/534 nm) of NBD was detected mainly in the green channel (490 nm/528 nm) of a Delta Vision RT Deconvolution Microscope. The fluorescence emission spectrum of Dox (Excitation/Emission=540 nm/600 nm, Pierce, Rockford, Ill.) allowed it to be visualized in the red channel (560 nm/617 nm) of a Delta Vision RT Deconvolution Microscope. In the study, the prostate LNCaP and PC3 cell lines were grown in 8-well microscope chamber slides in RPMI-1640 and Ham's F-12K medium, respectively, both supplemented with 100 units/ml aqueous penicillin G, 100 μg/ml streptomycin, and 10% FBS (fetal bovine serum) at concentrations to allow 70% confluence in 24 hours (i.e., 40,000 cells/cm²). On the day of experiments, cells were washed with pre-warmed PBS buffer and incubated with pre-warmed fresh media for 30 minutes before adding NP(NBD)-Apt(Dox) targeted particles with a final dye concentration of approximately 1 μg/ml (n=4). Cells were incubated with the targeted particles for 2 hours at 37° C., washed two times with PBS (300 μA per well), fixed with 4% formaldehyde, and mounted with non-fluorescent mounting medium DAPI (Cector Laboratory, Inc. Burlingame, Calif.). The cells were then imaged using a Delta Vision RT Deconvolution Microscope.

MTT Cell Viability Assay

Prostate LNCaP and PC3 cell lines were grown in 24-well plates in RPMI-1640 and Ham's F-12K medium, respectively, both supplemented with 100 units/ml aqueous penicillin G, 100 μg/ml streptomycin, and 10% FBS (fetal bovine serum) at concentrations to allow 70% confluence in 24 hours (i.e., 40,000 cells/cm²). On the day of experiments, cells were washed with pre-warmed PBS buffer and incubated with pre-warmed fresh media for 30 minutes before adding NP(Dtxl)-Apt(Dox) targeted particles with a final drug concentration of approximately 1 μg/ml (n=4). Three other systems, NP-Apt targeted particles carrying Dtxl alone [NP(Dtxl)-Apt], Dox alone [NP-Apt(Dox)], and no drug [NP-Apt], were chosen as controls for both cell lines. Cells were incubated with the targeted particles for 6 hours at 37° C., washed two times with PBS (1 ml per well), and then incubated in fresh growth media for a total of 72 hours. Cell viability was assessed colorimetrically with the MTT reagent (ATCC) following the standard protocol provided by the manufacturer.

### Results

Formulation of NP(Dxtl)-Apt(Dox) Targeted Particles

As illustrated in FIG. 8, using docetaxel (Dtxl) as a model small molecule hydrophobic drug; doxorubicin (Dox) as a model intercalating hydrophilic drug; the A10 RNA Apt which binds to the PSMA on the surface of PCa cells as a model aptamer targeting moiety (Lupold et al., 2002, Cancer Res., 62:4029); and poly(D,Llactic-co-glycolic acid)-block-poly(ethylene glycol) (PLGA-b-PEG) block copolymer as a model controlled release polymer system, targeted particles were developed (NP[Dxtl]-Apt[Dox]) that can co-deliver Dox and Dtxl to PSMA expressing PCa cells intracellularly. The A10 PSMA Apt is a 57 base pair nuclease-stabilized 2′-fluoropyrimidine RNA molecule with a single 5′-CG-3′ sequence in its predicted double stranded stem region that is the preferred binding site of Dox (Chaires et al., 1990, Biochemistry, 29:6145). Incubation of Dox with the A10 PSMA aptamer results in formation of a reversible physical conjugate, with a final Dox:Apt stoichiometry of 1:1.1, consistent with the intercalation of the Dox into a single CG sequence present in this aptamer (Bagalkot et al., 2006, Angew. Chem. Int. Ed., 45:8149). The present invention encompasses the recognition that the conjugation of Dox-loaded aptamer with Dtxl-encapsulated polymeric NPs results in targeted particle vehicles for targeted delivery of Dox and Dtxl (two chemotherapeutics with differing water solubility properties).

The biocompatible and biodegradable PLGA-b-PEG copolymer was used to formulate Dtxl encapsulated NPs (approximately 1% Dtxl by weight) with a diameter of 62±1.5 nm using the nanoprecipitation method (Bagalkot et al., 2006, Angew. Chem. Int. Ed., 45:8149; Cheng et al., 2007, Biomaterials, 28:869). NP surfaces were functionalized with the A10 PSMA Apt that was preloaded with Dox. The resulting targeted NP-Apt targeted particle carries and releases both Dtxl and Dox.

Drug Loading Efficiency of and Release Rate from NP(Dxtl)-Apt(Dox) Targeted Particles

Drug loading efficiency and release rate of Dtxl and Dox from the NP-Apt system was determined in PBS at 37° C. under gentle stirring. The drug content in solution was assayed over time and quantified using high performance liquid chromatography (HPLC). The data suggest that the relative carrying capacity of Dtxl to Dox in each NP-Apt targeted particle is approximately 9:1 (molar ratio), respectively, a property that can be tuned by controlling the total amount of each drug in the formulation process. Release profiles of Dtxl and Dox from NP(Dxtl)-Apt(Dox) targeted particles are shown in FIG. 8B. Approximately 50% and 80% of the initial dose of Dtxl was released from the polymeric core of the NP-Apt during the first 6 and 25 hours, respectively. Conversely, Dox release from the aptamer component of the NP-Apt was relatively fast, such that 50% and 80% of intercalated Dox was released within 4 and 6 hours, respectively. Without wishing to be bound by any one theory, the difference in release rates between Dtxl and Dox may be attributed to their relative hydrophobicity and their different loading mechanisms. For example, the former has low water solubility (0.025 mg/l) and is readily encapsulated within the hydrophobic core of NPs. The release of Dtxl depends on diffusion through the polymer matrix and on the hydrolysis of the PLGA polymer. Conversely, Dox is more soluble in water (10 g/l) and is more exposed to the aqueous solution, with release requiring only dissociation from the surface-bound aptamer.

Co-Delivery of Dxtl and Dox to Target Cells

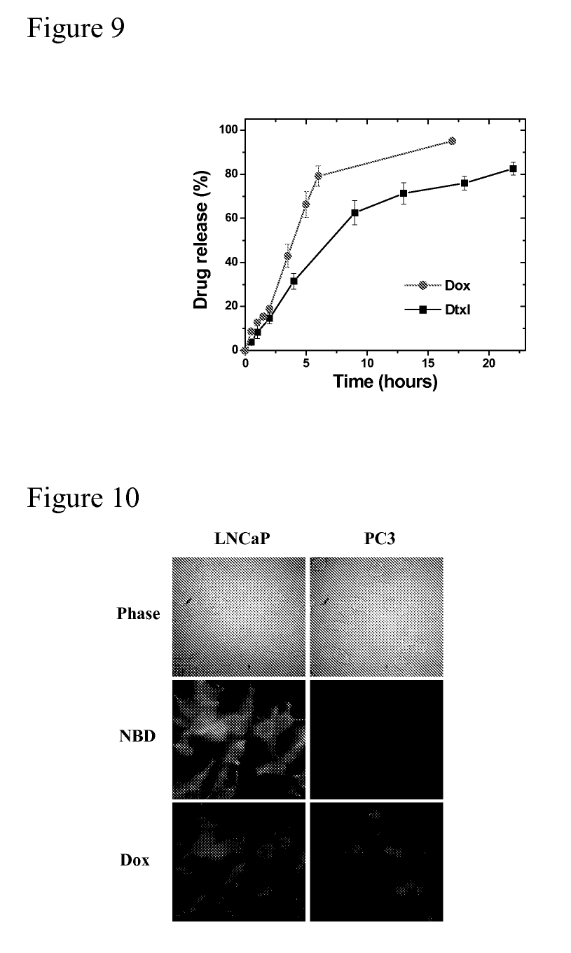

The present invention demonstrates that NP(Dxtl)-Apt(Dox) targeted particles are capable of co-delivering Dtxl and Dox selectively to target cells. LNCaP prostate adenocarcinomas, which express the PSMA antigen on their plasma membrane, were chosen as the target cell line for in vitro testing; PC3 prostate adenocarcinomas, which do not express the PSMA antigen, were employed as a negative control (Farokhzad et al., 2006, Proc. Natl. Acad. Sci., USA, 103:6315). To visualize cell uptake of drugs using fluorescence microscopy, a hydrophobic fluorescent probe, NBD-cholesterol (22-(N-(7-nitrobenz-2-oxa-1,3-diazol-4-yl)amino)-23,24-bisnor-5-cholen-3β-ol) (excitation/emission=460 nm/534 nm), was encapsulated inside PLGA-b-PEG NPs as an analog of a hydrophobic drug, while taking advantage of the fluorescence emission spectrum of Dox, which is in the red region to track the delivery of Dox. FIG. 9 shows that both NBD and Dox were effectively delivered into LNCaP cells by NP(Dxtl)-Apt(Dox) targeted particles. Virtually no NBD was delivered into PC3 cells, and the relatively small amount of Dox signal appearing in the PC3 nucleus may represent a portion of free Dox released from the NP(Dxtl)-Apt(Dox) targeted particles during incubation with cells, consistent with the observation that Dox release from the conjugates is relatively fast.

Cytotoxicity of NP(Dxtl)-Apt(Dox) Targeted Particles

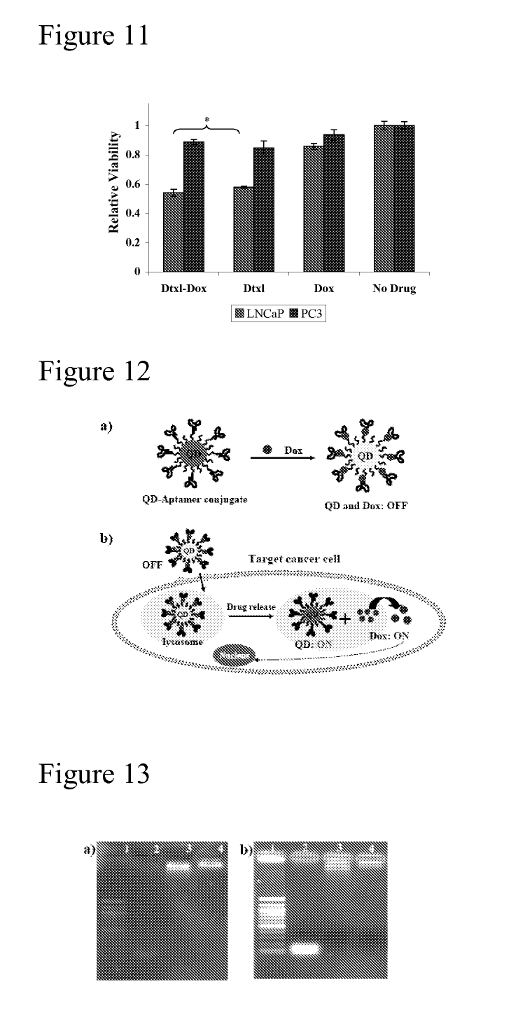

After having confirmed the feasibility of using NP(Dxtl)-Apt(Dox) targeted particles to co-deliver model hydrophobic and intercalating hydrophilic drugs to target cells, the in vitro cellular cytotoxicity of targeted particles carrying (i) both Dtxl and Dox [NP(Dtxl)-Apt(Dox)], (ii) Dtxl alone [NP(Dtxl)-Apt], (iii) Dox alone [NP-Apt(Dox)], or (iv) no drug [NP-Apt] to LNCaP and PC3 cell lines was examined. The results of the MTT cell proliferation assay (FIG. 11) show that for LNCaP cells treated with the same dose of drugs, NP(Dxtl)-Apt(Dox) targeted particles are more cytotoxic than all controls. Relative cell viability of NP(Dxtl)-Apt(Dox) is 54% in contrast to 58%, 86%, and 100% of using NP(Dtxl)-Apt, NP-Apt(Dox), and NP-Apt respectively. A one-sided, two sample t-test with equal variances was used to confirm that the observed differences between NP(Dxtl)-Apt(Dox) and NP(Dtxl)-Apt were statistically meaningful (p=0.029, n=4). Equality of variances was confirmed by F-test. Thus, the present invention encompasses the recognition that co-delivery of Dtxl and Dox may be more efficient than treating cells with the same amount of a single drug. The synergistic effects between the two chemotherapeutic drugs were obtained but did not reach a statistically significant level in this study. Without wishing to be bound by any one theory, one possible reason is that the molar ratio of Dtxl to Dox carried by each NP-Apt targeted particle is, on average, 9:1. The present invention encompasses the recognition that an aptamer with rich CG bases might enhance Dox carrying capacity, thereby, enhancing synergistic effects between drugs. The relative lack of toxicity on PC3 cells confirms the specificity of the NP(Dtxl)-Apt(Dox) targeted particle system, although some pre-released drugs during the period of sample preparation induced cell apoptosis (consistent with what was shown in FIG. 9).

### Example 3

### Quantum Dot-Aptamer Conjugates for Synchronous Cancer Imaging and Therapy Based on Bi-Fluorescence Resonance Energy Transfer

### Materials and Methods

Materials

Carboxyl core-shell CdSe/ZnS QD was obtained from Evitag (Troy, N.Y.), and Dox was obtained from Sigma-Aldrich (St. Louis, Mo.). Molecular biology buffers were purchased from Boston BioProducts (Worcester, Mass.). Tissue culture reagents and the LNCaP cell line were obtained from American Type Culture Collection (Manassas, Va.). All reagents were analytical grade or above and used as received, unless otherwise stated RNA aptamer (sequence: 5′-NH₂-spacer-[GGG/AGG/ACG/AUG/CGG/AUC/AGC/CAU/GUU/UAC/GUC/ACU/CCU/UGU/CAA/UCC/UCA/UCG/GCiT-3′(SEQ ID NO.: 3)] with 2′-fluoro pyrimidines, a 5′-amino group attached by a hexaethyleneglycol spacer and a 3′-inverted T cap) was custom synthesized by RNA-TEC (Leuven, Belgium) at a purity above 90%.

Formulation of QD-Apt Targeted Particles

The final QD-Apt-Dox conjugate for further experiments was made as follows: After conjugation of 5′—NH₂-modified A10 PSMA aptamers to QD (QD:Apt molar ratio of 1:10) and quenching of unreacted carboxyl on QD surface with ethanol amine (as described below), QD-Apt conjugates were purified using a 100K spin filter in order to remove the unbound aptamers. Purification was confirmed by agarose gel analysis.

40 μl (0.6 nM) carboxyl core-shell CdSe/ZnS quantum dots (QDs) (Evitag, Dunedin, Fla.) were first activated by incubating with 60 μl (50 mM) 1-ethyl-3-(3-dimethylaminopropyl) carbodiimide (EDC), and 30 μl (25 mM) N-hydroxysuccinimide (NHS) for 15 minutes under gentle stirring. The resulting N-hydroxysuccinimide-activated QDs were covalently linked to 5′—NH₂ modified A10 PSMA aptamers (QD:Apt molar ratio of 1:10). The mixture was reacted with slow rotation for 1 hour, and ethanol amine (100 mM) was added for 2 hours to quench unreacted carboxyls on the QD surface. QD-Apt conjugates were purified using a 100K spin filter in order to remove the unbound aptamers. The final QD-Aptamer was washed, resuspended in PBS, and characterized using gel electrophoresis.

Fluorescence Quenching of QD-Apt-Dox Targeted Particles

Dox was loaded onto the QD-Apt through titration based method. Briefly, purified QD-Apt conjugates (0.1 nM) were suspended in DNase RNase free water, followed by adding Dox with increasing molar ratios of 0.1, 0.3, 0.6, 1.0, 1.5, 2.1, 2.8, 3.5, 4.5, 5.5, 7.0, and 8.0. After each addition of Dox, the solution was mixed by vortexing for 30 minutes, and the fluorescence spectrum of the QDs was measured using Schimadzu RF-PC100 spectrofluorophotometer with an excitation wavelength of 350 nm and a recorded emission range of 440-560 nm. From the fluorescence spectra it was found that at 5.5 mole ratio of Dox the maximum quenching of QD was seen, and this was considered as maximum loading of Dox to QD-aptamer surface. For further in vitro experiments, this mole ratio was followed to make final Dox loaded QD-Apt targeted particle. In order to monitor the quenching effect on Dox, the Dox suspension (10 μM) was incubated with purified QD-Apt conjugates with increasing molar rations of 0.02, 0.04, 0.07, 0.09, 0.12, 0.14, and 0.16. Before measuring the fluorescence spectrum of Dox, the mixture suspension was incubated for 30 minutes. The excitation and emission range of Dox was 480 nm and 520-640 nm respectively.

Fluorescence Imaging Measurement

Prostate cancer cell lines LNCaP and PC3 cells (5,000 cells/ml) were grown in S-well microscope chamber slides in RPMI-1640 and Ham's F-12K medium, respectively, both supplemented with 100 units/ml aqueous penicillin G, 100 μg/mL streptomycin, and 10% FBS (fetal bovine serum) at concentrations to allow 70% confluence in 24 hour. On the day of experiments, cells were washed with pre-warmed PBS buffer and incubated with pre-warmed fresh media for 30 minutes before adding QD-Apt-Dox conjugate (100 nM) (n=4). Cells were incubated with the conjugates for 30 minutes at 37° C., washed two times with PBS (300 μl per well). For target binding experiments, cells were then fixed with 4% formaldehyde, mounted with non-fluorescent mounting medium DAPI (Cector Laboratory, Inc. Burlingame, Calif.), and imaged using confocal laser scanning microscopy (Carl Zeiss LSM 510, DAPI long pass filter set was used for QD imaging, and rhodamine filter set was used for Dox imaging). For time-dependent imaging experiments, cells were further incubated for 0 hours or 1.5 hours before fixing, mounting, and imaging.

MTT Cell Viability Assay

The prostate LNCaP and PC3 cell lines were grown in 24-well plates in RPMI-1640 and Ham's F-12K medium, respectively, both supplemented with 100 units/ml aqueous penicillin G, 100 μg/ml streptomycin, and 10% FBS (fetal bovine serum) at concentrations to allow 70% confluence in 24 hours (i.e., 40,000 cells/cm²). On the day of experiments, cells were washed with pre-warmed PBS buffer and incubated with pre-warmed fresh media for 30 minutes before adding QD-Apt-Dox targeted particles (100 nM), free QDs (100 nM), or free Dox (5 μM). Cells were incubated with the conjugates for 3 hours at 37° C., washed two times with PBS (1 ml per well), and incubated in fresh growth media for a total of 72 hours. Cell viability was assessed colorimetrically with the MTT reagent (ATCC) following the standard protocol provided by the manufacturer. The absorbance was read with a microplate reader at 570 nm.

### Results

Formulation of QD-Apt-Dox Targeted Particles

The amine terminated A10 RNA aptamer was conjugated to the surface of carboxyl terminated QDs using 1-ethyl-3-(3-dimethylaminopropyl)carbodiimide (EDC) and N-hydroxysuccinimide (NHS) activation chemistry. Gel electrophoresis (FIG. 13) showed that QD-Apt targeted particles were formed, and on average, each targeted particle carries 10 aptamers. Nonspecifically bound aptamers were efficiently washed off using centrifugal filtration. After forming QD-Apt targeted particles, the extra carboxyl groups present on QD surface were subsequently quenched using ethanol amine.

QD-Apt targeted particles were incubated with Dox to form QD-Apt-Dox targeted particles by intercalating Dox into the CG sequence present in PSMA aptamer (Bagalkot et al., 2006, Angew. Chem. Int. Ed., 45:8149). Since the extra negative charges present on CdSe/ZnS core-shell QD surface have been passivated, nonspecific binding of positively charged Dox to QD surfaces due to electrostatic attraction is negligible.

QD-Apt-Dox Targeted Particle Forms a Bi-FRET System

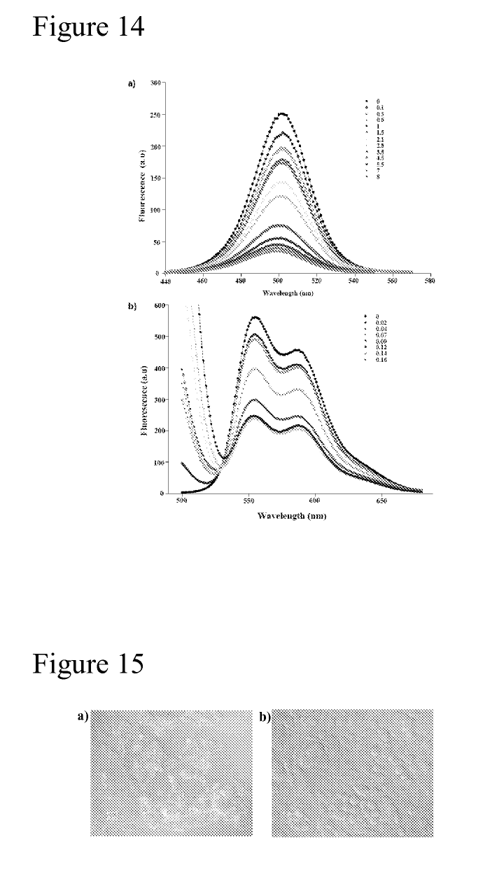

The anthracycline class of drugs has fluorescence properties (Valentini et al., 1985, Farmaco [Sci], 40:377; Haj et al., 2003, Chem. Biol. Interact., 145:349); for example, Dox can be effectively excited by absorbing photons with a wavelength of 480 nm, and Dox gives fluorescence emission in the range of 520-640 nm. Thus, the present invention encompasses the recognition that Dox can be a photon acceptor of CdSe/ZnS QD490, which emits fluorescence at the range of 470-530 nm with an excitation at 350 nm. In addition, the fluorescence emission from anthracycline class of drugs, including Dox, can be quenched after intercalation into DNA (Valentini et al., 1985, Farmaco [Sci], 40:377; Haj et al., 2003, Chem. Biol. Interact., 145:349). Recently, the inventors have reported that PSMA aptamer is able to quench Dox (Bagalkot et al., 2006, Angew. Chem. Int. Ed., 45:8149). Thus, the present invention encompasses the recognition that the QD-Apt-Dox targeted particles forms a Bi-FRET system; a donor-acceptor model FRET between QD and Dox; and a donor-quencher model FRET between Dox and PSMA aptamer. To examine whether such FRET systems occur in practice, fluorescence spectroscopy was used to monitor the binding of Dox to QD-Apt targeted particles. Sequential decreases in the fluorescence emission spectrum of QDs were observed when a fixed concentration of QD-Apt targeted particles was incubated with an increasing molar ratio of Dox (FIG. 14A). This result suggests that Dox binding indeed causes energy transfer from QD to Dox which diminishes QD emission. Minimal emission was achieved when, on average, 8 Dox bind to each QD-Apt targeted particle, which is consistent with the fact that, on average, each conjugate carries 10 aptamers and each PSMA aptamer carries a maximum of 1 Dox. FIG. 14B shows a similar sequential decrease of Dox emission when a fixed concentration of Dox was incubated with an increasing molar ratio of QD-Apt targeted particles. This observation confirms that the fluorescence emission of Dox can be quenched by an aptamer into which Dox intercalated. Therefore, the QD-Apt-Dox targeted particle forms a Bi-FRET system that has potential in cellular imaging with ultra specificity and sensitivity.

Specificity of Imaging Prostate Cancer Cells Using QD-Apt-Dox Targeted Particles

To evaluate the specificity of imaging prostate cancer (PCa) cells using QD-Apt-Dox targeted particles, LNCaP prostate adenocarcinomas, which express the PSMA antigen on their plasma membrane, were used as the target cancer cell line for in vitro testing. PC3 prostate adenocarcinomas, which do not express the PSMA antigen, were employed as a negative control (Farokhzad et al., 2006, Proc. Natl. Acad. Sci., USA, 103:6315). The fact that QD fluorescence was recovered after Dox was released from the conjugates allowed for visualization of cell uptake of the targeted particles using confocal laser scanning microscopy. Both cell lines were incubated with 100 nM Dox saturated QD-Apt-Dox targeted particles for 0.5 hours at 37° C. followed by copious washing to remove unbound conjugates. FIG. 15 shows that QD-Apt-Dox targeted particles were effectively delivered into LNCaP cells, while few conjugates were taken up by PC3 cells. Since the size of QD-Apt-Dox targeted particles is about 30 nm, they were capable to reach nuclei and demonstrate both nuclear and cytosolic staining for LNCaP cells (FIG. 15A). However, the conjugates failed to image PC3 cells because very few of them were delivered into PC3 cells. This is consistent with the lack of PSMA antigen present on these cells.

Time Profile of Imaging Prostate Cancer Cells Using QD-Apt-Dox Targeted Particles

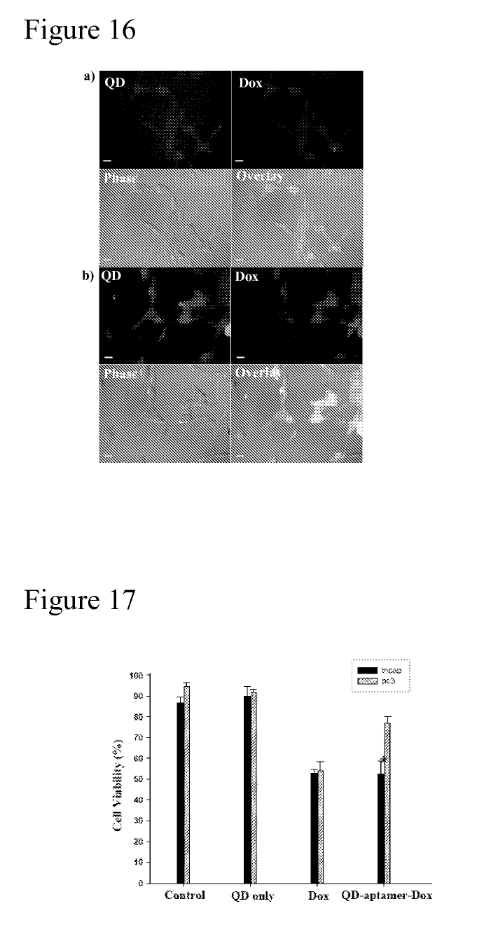

To further assess the sensitivity of imaging PCa cells using QD-Aptamer-Dox conjugate, a time profile of the fluorescence intensities of QD and Dox after the conjugates were taken up by LNCaP cells was investigated using confocal laser scanning microscopy. LNCaP cells were incubated with 100 nM Dox saturated QD-Apt-Dox targeted particles for 0.5 hours at 37° C. and washed twice with PBS to remove free conjugates. The cells were imaged either immediately (QD fluorescence) or after 1.5 hours of further incubation (Dox fluorescence). The data show that both QD and Dox mostly remained in the “OFF” state at 0 hours post-incubation, as only faint fluorescence signals were observed (FIG. 16A). Without wishing to be bound by any one theory, this could be because the majority of Dox remained in the targeted particles. However, after 1.5 hours post-incubation, more Dox was released from the targeted particles. Consequently, a substantial fluorescence increase appeared for both QD and Dox, indicating that they were turned to the “ON” state (FIG. 16B). Moreover, both QD and Dox gave very sharp images of the cancer cells with low background noise, which suggests that QD-Apt-Dox targeted particles are sensitive to detect cancer cells on single-cell level. The present invention encompasses the recognition that this could utilized in early stage tumor diagnosis, when the amount of cancerous cells is usually small.

Cytotoxicity of QD-Apt-Dox Targeted Particles

After having confirmed the feasibility of using QD-Apt-Dox targeted particles for cancer imaging, the in vitro cellular cytoxtoxicity of targeted particles to LNCaP and PC3 cell lines was determined as compared to QD alone and Dox alone. The MTT cell proliferation assay results (FIG. 17) demonstrated that while the cytotoxicity of free Dox was equipotent against LNCaP and PC3 cells, the cytotoxicity of QD-Apt-Dox targeted particles was enhanced against targeted LNCaP cells as compared to nontargeted PC3 cells (cellular viability: LNCaP 52.5±1.6% versus PC3 77.2±3.1%; mean±SE, n=3; probability value p<0.005). The data show that the cytotoxicity of QD-Apt-Dox targeted particles was nearly equipotent to that of free Dox. Free QD without Apt or Dox had no inherent cytotoxicity to LNCaP and PC3 cells (FIG. 17), and the inventors have previously reported that free PSMA aptamer has no cytotoxicity to either cell line. Thus, the present invention encompasses the recognition that cytotoxicity of QD-Apt-Dox targeted particles stems from the released Dox molecules after endocytic uptake by LNCaP cells. Without wishing to be bound by any one theory, Dox release may be induced by physical dissociation of Dox from the targeted particles and biodegradation of PSMA aptamer by endonucleases in the lysosomes. In contrast to the significant cytotoxicity of QD-Apt-Dox targeted particles to the LNCaP cells, targeted particles had a less pronounced cytotoxic effect to PC3 cells, which the inventors attributed to the lack of PSMA antigen present on their plasma membrane (consistent with what was shown in FIG. 15).

### EQUIVALENTS AND SCOPE

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a nanoparticle” includes a plurality of such nanoparticle, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any aptamer, any disease, disorder, and/or condition, any linking agent, any method of administration, any therapeutic application, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

1. A complex, comprising:
a nucleic acid targeting moiety; and
a therapeutic agent, wherein the therapeutic agent is capable of associating with the base pairs of the nucleic acid targeting moiety.

2. The complex of claim 1, wherein the therapeutic agent is non-covalently associated with the nucleic acid targeting moiety.

3. The complex of claim 1, wherein the nucleic acid targeting moiety is an aptamer.

4-17. (canceled)

18. The complex of claim 2, wherein the specific binding of the nucleic acid targeting moiety to a target results in delivery of the therapeutic agent to target cells, and wherein the target comprises a protein.

19. The complex of claim 1, wherein the target is selected from the group consisting of cell surface receptors, integrins, transmembrane proteins, ion channels, membrane transport proteins, intracellular proteins, soluble proteins, small molecules, tumor markers, characteristic portions thereof, and combinations thereof.

20. (canceled)

21. The complex of claim 2, wherein the specific binding of the nucleic acid targeting moiety to a target results in delivery of the therapeutic agent to target cells, and wherein the target is a prostate cancer specific marker.

22. (canceled)

23. The complex of claim 21, wherein the target is PMSA.

24-27. (canceled)

28. The complex of claim 2, wherein the specific binding of the nucleic acid targeting moiety to a target results in delivery of the therapeutic agent to target cells, and wherein the specific binding of the nucleic acid targeting moiety to the target depends on the three dimensional characteristics of the targeting moiety.

29. The complex of claim 1, wherein the therapeutic agent intercalates between the base pairs of the nucleic acid targeting moiety.

30. The complex of claim 1, wherein the therapeutic agent is an anthracycline.

31-44. (canceled)

45. A targeted particle, comprising:
a particle; and
a complex;
wherein the complex comprises:
a nucleic acid targeting moiety; and
a therapeutic agent, wherein the therapeutic agent is capable of intercalating between the base pairs of the nucleic acid targeting moiety.

46. The targeted particle of claim 45, wherein the particle comprises a polymeric matrix, and wherein the polymeric matrix comprises a polyester.

47-50. (canceled)

51. The targeted particle of claim 46, wherein the polyester is selected from the group consisting of PLGA, PLA, PGA, polycaprolactone, and polyanhydrides.

52. (canceled)

53. The targeted particle of claim 45, wherein the polymeric matrix comprises two or more polymers.

54-56. (canceled)

57. The targeted particle of claim 53, wherein at least one polymer is polyethylene glycol (PEG).

58. The targeted particle of claim 45, wherein the polymeric matrix comprises a copolymer of two or more polymers.

59-112. (canceled)

113. The targeted particle of claim 45, wherein the complex is non-covalently associated with the particle.

114-143. (canceled)

144. A method of preparing a targeted particle comprising:
providing a nucleic acid targeting moiety;
providing a therapeutic agent, wherein the therapeutic agent is capable of intercalating between the base pairs of the nucleic acid targeting moiety;
mixing the nucleic acid targeting moiety with the therapeutic agent to prepare a complex;
providing a polymer, wherein the polymer comprises a polyester;
producing a particle comprising a polymeric matrix; and
associating the particles with the complex;
wherein the polymer comprises a polyester, and wherein the nucleic acid targeting moiety targets prostate cancer cells.

145. The method of claim 144, further comprising a step of purifying the particles.

146. The method of claim 144, further comprising a step of isolating particles of a predetermined size.

147-148. (canceled)


SEQUENCE LISTING (3 sequences)

SEQ ID NO: 1 - RNA, 71 nt, Artificial Sequence
  Synthetic A10 Aptamer
gggaggacgaugcggaucagccauguuuacgucacuccuugucaauccucaucggcagacgacucgcccga

SEQ ID NO: 2 - RNA, 70 nt, Artificial Sequence
  Synthetic A9 Aptamer
gggaggacgaugcggaccgaaaaagaccugacuucuauacuaagucuacguucccagacgacucgcccga

SEQ ID NO: 3 - RNA, 56 nt, Artificial Sequence
  Synthetic Aptamer
gggaggacgaugcggaucagccauguuuacgucacuccuugucaauccucaucggc